(19) United States
(12) Patent Application Publication
(10) Pub. No.: US 2026/0000772 A1
(43) Pub. Date: Jan. 1, 2026

(54) NONSTEROIDAL ANTI-INFLAMMATORY DRUG COMPLEXES AND METHODS OF MAKING AND USING

(71) Applicant: Areca BSC Development, LLC, Statham, GA (US)

(72) Inventors: Jeff BRYANT, Statham, GA (US); H. Greg Thomas, Carrolton, GA (US); Roy Bryant, Dahlonega, GA (US)

(21) Appl. No.: 19/257,417

(22) Filed: Jul. 1, 2025

Related U.S. Application Data
(60) Provisional application No. 63/666,298, filed on Jul. 1, 2024.

(51) Int. Cl. A61K 47/58; A61K 9/00; A61K 9/14; A61K 31/192; A61K 47/02; A61K 47/10; A61K 47/12; A61K 47/14; A61K 47/18; A61K 47/26; A61K 47/36

(52) U.S. Cl. CPC A61K 47/585; A61K 9/0095; A61K 9/146; A61K 31/192; A61K 47/02; A61K 47/10; A61K 47/12; A61K 47/14; A61K 47/183; A61K 47/26; A61K 47/36

(57) ABSTRACT

Disclosed herein are novel IEX-NSAID complexes comprising a nonsteroidal anti-inflammatory drug (NSAID) or a salt thereof bound to an ion-exchange (IEX) resin, as well as liquid dosage forms thereof, and their use for treating, ameliorating, preventing, or reducing the severity of numerous conditions, or their symptoms, for with NSAID are the prescribed treatment. Also disclosed herein are methods for making the aforementioned IEX-NSAID complexes, and liquid dosage forms thereof.

### CROSS-REFERENCE TO RELATED APPLICATIONS

This application claims the benefit of U.S. Provisional Application No. 63/666,298 filed Jul. 1, 2024, the contents of which are hereby incorporated by reference in their entirety.

### BACKGROUND

Nonsteroidal anti-inflammatory drugs (“NSAIDs”) are a class of therapeutic agents that can be used to reduce pain and aches, decrease inflammation, reduce fever, and prevent blood clots, among other functions. The popularity of NSAIDs is evidenced by their sizeable global market, which was $19.6 billion USD as of 2021, and is expected to grow to $31.5 billion USD by 2030.

Generally, NSAIDs are defined structurally by an acidic moiety, e.g., a carboxylic acid, an enol, and the like, appended to a planar aromatic functional group. As a consequence of their structure, NSAIDs often have poor solubility in aqueous media. This can be detrimental because NSAIDs are often administered orally to subjects, and a poor aqueous solubility may require a large volume to administer an effective dose of the NSAID, which can cause discomfort and/or be impractical for the subject taking the NSAID, and therefore clinically undesirable. Accordingly, drug formulators, compounding pharmacists, physicians, and the like, prefer concentrated dosage forms of the NSAID, which can be achieved via various formulating techniques, such as converting the NSAID to a salt, including a sodium salt, a potassium salt, an alkylammonium salt, and the like.

Moreover, NSAIDs that contain naphthalene- or phenylacetic acid-motifs, e.g., naproxen, diclofenac, mefenamic acid, flufenamic acid, ibuprofen, ketoprofen, flurbiprofen, and the like, may also cause undesirable chemesthetic effects in the oral cavity and/or pharyngeal cavity when the NSAID is orally ingested. Such undesirable chemesthetic effects, can include, but are not limited to irritation, burning, stinging, pricking, itching, tingling, numbness, tickling, and the like. In addition, NSAIDs may also have undesirable taste profiles when the NSAID is orally ingested, including, for example, bitterness, saltiness, sourness, a metallic taste, a lye-like taste, and the like.

Because of their poor solubility (and thus large administration volume), undesirable chemesthetic effects, and/or undesirable taste profile, patient compliance with a prescribed orally administered NSAID regimen may be reduced. A reduction a patient compliance may be further exacerbated in subject populations sensitive to the aforementioned effects, such as older patients and children—groups that often require high patient compliance for expedient resolution of symptoms.

To address these shortcomings, the present inventors have developed a novel oral dosage form for NSAIDs that (i) utilize aqueous-soluble forms of NSAIDs to minimize the administration volume, (ii) reduces/eliminates the undesirable chemesthetic effects that occur when NSAIDs are orally ingested, and (iii) reduces/eliminates the undesirable taste profile associated with orally administered NSAIDs. To this end, the present inventors have developed novel oral dosage forms comprising ion-exchange (“IEX”) resin-bound NSAIDs (“IEX-NSAID complexes”).

In the drug formulation arts, researchers have contemplated the use of IEX resins in oral dosage forms for non-NSAID formulations to impart delayed-, controlled-, or slowed release of the therapeutic payload. IEX resins, however, have not been used with NSAIDs, largely because successful treatment or amelioration of the conditions or symptoms discussed herein often requires a rapid delivery of the NSAID.

Herein, for the first time, the inventors report novel liquid oral dosage forms comprising IEX-NSAID complexes, which can eliminate the undesirable chemesthetic effects and taste profiles associated with NSAIDs. The inventors present results demonstrating surprising and unexpectedly rapid dissolution of the NSAID from its bound state in the IEX-NSAID complexes disclosed herein. The inventors developed an exemplary liquid oral dosage form comprising a cholestyramine-naproxen sodium complex that eliminates the undesirable chemesthetic effects associated with naproxen and rapidly releases naproxen from its resin-bound state. This finding is novel. As such, the oral dosage forms disclosed herein have significant clinical relevance—by rapidly delivering NSAIDs to a subject without chemesthetic effects, the effective treatment, amelioration, prevention, and/or reduction in the severity of multiple conditions or symptoms thereof can be realized.

### SUMMARY

Embodiments of the present disclosure relate to novel IEX-NSAID complexes comprising a nonsteroidal anti-inflammatory drug (“NSAID”) or a salt thereof bound to an ion-exchange (“IEX”) resin.

Embodiments of the present disclosure relate to novel IEX-NSAID complexes comprising naproxen or a salt thereof bound to an IEX resin.

Embodiments of the present disclosure relate to novel IEX-NSAID complexes comprising naproxen sodium bound to an IEX resin.

Embodiments of the present disclosure relate to novel IEX-NSAID complexes comprising an NSAID or a salt thereof bound to an anion exchange resin.

Embodiments of the present disclosure relate to novel IEX-NSAID complexes comprising naproxen or a salt thereof bound to an anion exchange resin.

Embodiments of the present disclosure relate to novel IEX-NSAID complexes comprising naproxen sodium bound to an anion exchange resin.

Embodiments of the present disclosure relate to novel IEX-NSAID complexes comprising an NSAID or a salt thereof bound to cholestyramine.

Embodiments of the present disclosure relate to novel IEX-NSAID complexes comprising naproxen or a salt thereof bound to cholestyramine.

Embodiments of the present disclosure relate to novel IEX-NSAID complexes comprising naproxen sodium bound to cholestyramine.

Embodiments of the present disclosure relate to methods of making novel IEX-NSAID complexes comprising an NSAID or a salt thereof bound to an IEX resin.

Embodiments of the present disclosure relate to methods of making novel IEX-NSAID complexes comprising naproxen or a salt thereof bound to an IEX resin.

Embodiments of the present disclosure relate to methods of making novel IEX-NSAID complexes comprising naproxen sodium bound to an IEX resin.

Embodiments of the present disclosure relate to methods of making novel IEX-NSAID complexes comprising an NSAID or a salt thereof bound to an anion exchange resin.

Embodiments of the present disclosure relate to methods of making novel IEX-NSAID complexes comprising naproxen or a salt thereof bound to an anion exchange resin.

Embodiments of the present disclosure relate to methods of making novel IEX-NSAID complexes comprising naproxen sodium bound to an anion exchange resin.

Embodiments of the present disclosure relate to methods of making novel IEX-NSAID complexes comprising an NSAID or a salt thereof bound to cholestyramine.

Embodiments of the present disclosure relate to methods of making novel IEX-NSAID complexes comprising naproxen or a salt thereof bound to cholestyramine.

Embodiments of the present disclosure relate to methods of making novel IEX-NSAID complexes comprising naproxen sodium bound to cholestyramine.

Embodiments of the present disclosure relate to novel liquid dosage forms comprising an amount of an IEX-NSAID complex comprising an NSAID or a salt thereof bound to an IEX resin.

Embodiments of the present disclosure relate to novel liquid dosage forms comprising an amount of an IEX-NSAID complex comprising naproxen or a salt thereof bound to an IEX resin.

Embodiments of the present disclosure relate to novel liquid dosage forms comprising an amount of an IEX-NSAID complex comprising naproxen sodium bound to an IEX resin.

Embodiments of the present disclosure relate to novel liquid dosage forms comprising an amount of an IEX-NSAID complex comprising an NSAID or a salt thereof bound to an anion exchange resin.

Embodiments of the present disclosure relate to novel liquid dosage forms comprising an amount of an IEX-NSAID complex comprising naproxen or a salt thereof bound to an anion exchange resin.

Embodiments of the present disclosure relate to novel liquid dosage forms comprising an amount of an IEX-NSAID complex comprising naproxen sodium bound to an anion exchange resin.

Embodiments of the present disclosure relate to novel liquid dosage forms comprising an amount of an IEX-NSAID complex comprising an NSAID or a salt thereof bound to cholestyramine.

Embodiments of the present disclosure relate to novel liquid dosage forms comprising an amount of an IEX-NSAID complex comprising naproxen or a salt thereof bound to cholestyramine.

Embodiments of the present disclosure relate to novel liquid dosage forms comprising an amount of an IEX-NSAID complex comprising naproxen sodium bound to cholestyramine.

Embodiments of the present disclosure relate to methods of making novel liquid dosage forms comprising an amount of an IEX-NSAID complex comprising an NSAID or a salt thereof bound to an IEX resin.

Embodiments of the present disclosure relate to methods of making novel liquid dosage forms comprising an amount of an IEX-NSAID complex comprising naproxen or a salt thereof bound to an IEX resin.

Embodiments of the present disclosure relate to methods of making novel liquid dosage forms comprising an amount of an IEX-NSAID complex comprising naproxen sodium bound to an IEX resin.

Embodiments of the present disclosure relate to methods of making novel liquid dosage forms comprising an amount of an IEX-NSAID complex comprising an NSAID or a salt thereof bound to an anion exchange resin.

Embodiments of the present disclosure relate to methods of making novel liquid dosage forms comprising an amount of an IEX-NSAID complex comprising naproxen or a salt thereof bound to an anion exchange resin.

Embodiments of the present disclosure relate to methods of making novel liquid dosage forms comprising an amount of an IEX-NSAID complex comprising naproxen sodium bound to an anion exchange resin.

Embodiments of the present disclosure relate to methods of making novel liquid dosage forms comprising an amount of an IEX-NSAID complex comprising an NSAID or a salt thereof bound to cholestyramine.

Embodiments of the present disclosure relate to methods of making novel liquid dosage forms comprising an amount of an IEX-NSAID complex comprising naproxen or a salt thereof bound to cholestyramine.

Embodiments of the present disclosure relate to methods of making novel liquid dosage forms comprising an amount of an IEX-NSAID complex comprising naproxen sodium bound to cholestyramine.

Embodiments of the present disclosure relate to the use of novel IEX-NSAID complexes for treating, ameliorating, preventing, and/or reducing the severity of acute pain or a symptom thereof in a subject.

Embodiments of the present disclosure relate to the use of novel IEX-NSAID complexes for treating, ameliorating, preventing, and/or reducing the severity of chronic pain or a symptom thereof in a subject.

Embodiments of the present disclosure relate to the use of novel IEX-NSAID complexes for treating, ameliorating, preventing, and/or reducing the severity of acute inflammation or a symptom thereof in a subject.

Embodiments of the present disclosure relate to the use of novel IEX-NSAID complexes for treating, ameliorating, preventing, and/or reducing the severity of chronic inflammation or a symptom thereof in a subject.

Embodiments of the present disclosure relate to the use of novel IEX-NSAID complexes for treating, ameliorating, preventing, and/or reducing the severity of a fever or a symptom thereof in a subject.

Embodiments of the present disclosure relate to the use of novel IEX-NSAID complexes for treating, ameliorating, preventing, and/or reducing the severity of blood clots or a symptom thereof in a subject.

Embodiments of the present disclosure relate to the use of novel IEX-NSAID complexes for treating, ameliorating, preventing, and/or reducing the severity of a heart attack or a symptom thereof in a subject.

Embodiments of the present disclosure relate to the use of novel IEX-NSAID complexes for treating, ameliorating, preventing, and/or reducing the severity of a stroke or a symptom thereof in a subject.

Embodiments of the present disclosure relate to the use of novel IEX-NSAID complexes for treating, ameliorating, preventing, and/or reducing the severity of osteoarthritis or a symptom thereof in a subject.

Embodiments of the present disclosure relate to the use of novel IEX-NSAID complexes for treating, ameliorating, preventing, and/or reducing the severity of rheumatoid arthritis or a symptom thereof in a subject.

Embodiments of the present disclosure relate to the use of novel IEX-NSAID complexes for treating, ameliorating, preventing, and/or reducing the severity of headaches or a symptom thereof in a subject.

Embodiments of the present disclosure relate to the use of novel IEX-NSAID complexes for treating, ameliorating, preventing, and/or reducing the severity of migraines or a symptom thereof in a subject.

Embodiments of the present disclosure relate to the use of novel IEX-NSAID complexes for treating, ameliorating, preventing, and/or reducing the severity of gout or a symptom thereof in a subject.

Embodiments of the present disclosure relate to the use of novel IEX-NSAID complexes for treating, ameliorating, preventing, and/or reducing the severity of dysmenorrhea or a symptom thereof in a subject.

Embodiments of the present disclosure relate to the use of novel IEX-NSAID complexes treating, ameliorating, preventing, and/or reducing the severity of muscle stiffness or a symptom thereof in a subject.

Embodiments of the present disclosure relate to novel IEX-NSAID complexes for treating, ameliorating, preventing, and/or reducing the severity of ileus or a symptom thereof in a subject.

Embodiments of the present disclosure relate to the use of novel IEX-NSAID complexes for treating, ameliorating, preventing, and/or reducing the severity of macular edema or a symptom thereof in a subject.

Embodiments of the present disclosure relate to the use of novel IEX-NSAID complexes to temporarily relieve minor aches and pains due to arthritis.

Embodiments of the present disclosure relate to the use of novel IEX-NSAID complexes to temporarily relieve minor aches and pains due to muscular aches.

Embodiments of the present disclosure relate to the use of novel IEX-NSAID complexes to temporarily relieve minor aches and pains due to backache.

Embodiments of the present disclosure relate to the use of novel IEX-NSAID complexes to temporarily relieve minor aches and pains due to menstrual cramps.

Embodiments of the present disclosure relate to the use of novel IEX-NSAID complexes to temporarily relieve minor aches and pains due to headache.

Embodiments of the present disclosure relate to the use of novel IEX-NSAID complexes to temporarily relieve minor aches and pains due to toothache.

Embodiments of the present disclosure relate to the use of novel IEX-NSAID complexes to temporarily relieve minor aches and pains due to the common cold.

Embodiments of the present disclosure relate to the use of novel IEX-NSAID complexes to temporarily reduce fever.

Embodiments of the present disclosure relate to the use of novel liquid dosage forms comprising an amount of an IEX-NSAID complex for treating, ameliorating, preventing, and/or reducing the severity of acute pain or a symptom thereof in a subject.

Embodiments of the present disclosure relate to the use of novel liquid dosage forms comprising an amount of an IEX-NSAID complex for treating, ameliorating, preventing, and/or reducing the severity of chronic pain or a symptom thereof in a subject.

Embodiments of the present disclosure relate to the use of novel liquid dosage forms comprising an amount of an IEX-NSAID complex for treating, ameliorating, preventing, and/or reducing the severity of acute inflammation or a symptom thereof in a subject.

Embodiments of the present disclosure relate to the use of novel liquid dosage forms comprising an amount of an IEX-NSAID complex for treating, ameliorating, preventing, and/or reducing the severity of chronic inflammation or a symptom thereof in a subject.

Embodiments of the present disclosure relate to the use of novel liquid dosage forms comprising an amount of an IEX-NSAID complex for treating, ameliorating, preventing, and/or reducing the severity of a fever or a symptom thereof in a subject.

Embodiments of the present disclosure relate to the use of novel liquid dosage forms comprising an amount of an IEX-NSAID complex for treating, ameliorating, preventing, and/or reducing the severity of blood clots or a symptom thereof in a subject.

Embodiments of the present disclosure relate to the use of novel liquid dosage forms comprising an amount of an IEX-NSAID complex for treating, ameliorating, preventing, and/or reducing the severity of a heart attack or a symptom thereof in a subject.

Embodiments of the present disclosure relate to the use of novel liquid dosage forms comprising an amount of an IEX-NSAID complex for treating, ameliorating, preventing, and/or reducing the severity of a stroke or a symptom thereof in a subject.

Embodiments of the present disclosure relate to the use of novel liquid dosage forms comprising an amount of an IEX-NSAID complex for treating, ameliorating, preventing, and/or reducing the severity of osteoarthritis or a symptom thereof in a subject.

Embodiments of the present disclosure relate to the use of novel liquid dosage forms comprising an amount of an IEX-NSAID complex for treating, ameliorating, preventing, and/or reducing the severity of rheumatoid arthritis or a symptom thereof in a subject.

Embodiments of the present disclosure relate to the use of novel liquid dosage forms comprising an amount of an IEX-NSAID complex for treating, ameliorating, preventing, and/or reducing the severity of headaches or a symptom thereof in a subject.

Embodiments of the present disclosure relate to the use of novel liquid dosage forms comprising an amount of an IEX-NSAID complex for treating, ameliorating, preventing, and/or reducing the severity of migraines or a symptom thereof in a subject.

Embodiments of the present disclosure relate to the use of novel liquid dosage forms comprising an amount of an IEX-NSAID complex for treating, ameliorating, preventing, and/or reducing the severity of gout or a symptom thereof in a subject.

Embodiments of the present disclosure relate to the use of novel liquid dosage forms comprising an amount of an IEX-NSAID complex for treating, ameliorating, preventing, and/or reducing the severity of dysmenorrhea or a symptom thereof in a subject.

Embodiments of the present disclosure relate to the use of novel liquid dosage forms comprising an amount of an IEX-NSAID complex for treating, ameliorating, preventing, and/or reducing the severity of muscle stiffness or a symptom thereof in a subject.

Embodiments of the present disclosure relate to the use of novel liquid dosage forms comprising an amount of an IEX-NSAID complex for treating, ameliorating, preventing, and/or reducing the severity of ileus or a symptom thereof in a subject.

Embodiments of the present disclosure relate to the use of novel liquid dosage forms comprising an amount of an IEX-NSAID complex for treating, ameliorating, preventing, and/or reducing the severity of macular edema or a symptom thereof in a subject.

Embodiments of the present disclosure relate to the use of novel liquid dosage forms comprising an amount of an IEX-NSAID complex to temporarily relieve minor aches and pains due to arthritis.

Embodiments of the present disclosure relate to the use of novel liquid dosage forms comprising an amount of an IEX-NSAID complex to temporarily relieve minor aches and pains due to muscular aches.

Embodiments of the present disclosure relate to the use of novel liquid dosage forms comprising an amount of an IEX-NSAID complex to temporarily relieve minor aches and pains due to backache.

Embodiments of the present disclosure relate to the use of novel liquid dosage forms comprising an amount of an IEX-NSAID complex to temporarily relieve minor aches and pains due to menstrual cramps.

Embodiments of the present disclosure relate to the use of novel liquid dosage forms comprising an amount of an IEX-NSAID complex to temporarily relieve minor aches and pains due to headache.

Embodiments of the present disclosure relate to the use of novel liquid dosage forms comprising an amount of an IEX-NSAID complex to temporarily relieve minor aches and pains due to toothache.

Embodiments of the present disclosure relate to the use of novel liquid dosage forms comprising an amount of an IEX-NSAID complex to temporarily relieve minor aches and pains due to the common cold.

Embodiments of the present disclosure relate to the use of novel liquid dosage forms comprising an amount of an IEX-NSAID complex to temporarily relieve fever.

### BRIEF DESCRIPTION OF THE DRAWINGS

FIG. 1 Percentage of naproxen sodium, an NSAID, bound by cholestyramine resin, an IEX resin, in an aqueous solution following a mixing reaction with cholestyramine resin as a function of (i) the ratio of naproxen sodium to cholestyramine resin (mass to mass) in the aqueous solution, and (ii) duration of the mixing reaction (“Time of Mixing”). In FIG. 1, the initial naproxen sodium concentration in the aqueous solution was 22 mg/mL, the ratios of naproxen sodium to cholestyramine resin evaluated were in the range of 1:1 to 1:2 (naproxen sodium to cholestyramine resin), and the duration of the mixing reaction was in the range of 2 hrs to 6 hrs.

FIG. 2 Amount of naproxen sodium, an NSAID, remaining in an aqueous solution following a mixing reaction with cholestyramine resin, an IEX resin, as a function of (i) the ratio of naproxen sodium to cholestyramine resin (mass to mass) in the aqueous solution, and (ii) duration of the mixing reaction (“Time of Mixing”). In FIG. 2, the initial naproxen sodium concentration in the aqueous solution was 22 mg/mL, the ratios of naproxen sodium to cholestyramine resin evaluated were in the range of 1:1 to 1:2 (naproxen sodium to cholestyramine resin), and the duration of the mixing reaction was in the range of 2 hrs to 6 hrs.

FIG. 3 Percentage of naproxen sodium, an NSAID, bound by cholestyramine resin, an IEX resin, in an aqueous solution following a mixing reaction with cholestyramine resin as a function of (i) the ratio of naproxen sodium to cholestyramine resin (mass to mass) in the aqueous solution, and (ii) duration of the mixing reaction (“Time of Mixing”). In FIG. 3, the initial naproxen sodium concentration in the aqueous solution was 11 g of naproxen sodium per 100 g of water, the ratios of naproxen sodium to cholestyramine resin evaluated were in the range of 1:0.5 to 1:2 (naproxen sodium to cholestyramine resin), and the duration of the mixing reaction was in the range of 1 hr to 4 hrs.

FIG. 4 Amount of naproxen sodium, an NSAID, remaining in an aqueous solution following a mixing reaction with cholestyramine resin, an IEX resin, as a function of (i) the ratio of naproxen sodium to cholestyramine resin (mass to mass) in the aqueous solution, and (ii) duration of the mixing reaction (“Time of Mixing”). In FIG. 4, the initial naproxen sodium concentration in the aqueous solution was 11 g of naproxen sodium per 100 g of water, the ratios of naproxen sodium to cholestyramine resin evaluated were in the range of 1:0.5 to 1:2 (naproxen sodium to cholestyramine resin), and the duration of the mixing reaction was in the range of 1 hr to 4 hrs.

FIG. 5 Percentage of naproxen sodium, an NSAID, bound by cholestyramine resin, an IEX resin, in an aqueous solution following a mixing reaction with cholestyramine resin as a function of (i) the ratio of naproxen sodium to cholestyramine resin (mass to mass) in the aqueous solution, and (ii) duration of the mixing reaction (“Time of Mixing”). In FIG. 5, the initial naproxen sodium concentration in the aqueous solution was 11 g of naproxen sodium per 100 g of water, the ratios of naproxen sodium to cholestyramine resin evaluated were in the range of 1:1.25 to 1:5 (naproxen sodium to cholestyramine resin), and the duration of the mixing reaction was in the range of 0.5 hrs to 2 hrs.

FIG. 6 Amount of naproxen sodium, an NSAID, remaining in an aqueous solution following a mixing reaction with cholestyramine resin, an IEX resin, as a function of (i) the ratio of naproxen sodium to cholestyramine resin (mass to mass) in the aqueous solution, and (ii) duration of the mixing reaction (“Time of Mixing”). In FIG. 6, the initial naproxen sodium concentration in the aqueous solution was 11 g of naproxen sodium per 100 g of water, the ratios of naproxen sodium to cholestyramine resin evaluated were in the range of 1:1.25 to 1:1.5 (naproxen sodium to cholestyramine resin), and the duration of the mixing reaction was in the range of 0.5 hrs to 2 hrs.

FIG. 7 Percentage of naproxen sodium, an NSAID, bound by cholestyramine resin, an IEX resin, in an aqueous solution following a mixing reaction with cholestyramine resin as a function of (i) the ratio of naproxen sodium to cholestyramine resin (mass to mass) in the aqueous solution, and (ii) the initial naproxen sodium concentration in the aqueous solution. In FIG. 7, the initial naproxen sodium concentrations in the aqueous solution evaluated were 1.1 mg/mL (“110 mg Naproxen”) and 2.2 mg/mL (“220 mg Naproxen”), the ratios of naproxen sodium to cholestyramine resin evaluated were in the range of 1:0 to 1:4 (naproxen sodium to cholestyramine resin), and the duration of the mixing reaction was 1 hr.

FIG. 8 Amount of naproxen sodium, an NSAID, remaining in an aqueous solution following a mixing reaction with cholestyramine resin, an IEX resin, as a function of (i) the ratio of naproxen sodium to cholestyramine resin (mass to mass) in the aqueous solution, and (ii) the initial naproxen sodium concentration in the aqueous solution. In FIG. 8, the initial naproxen sodium concentrations in the aqueous solution evaluated were 1.1 mg/mL (“110 mg Naproxen”) and 2.2 mg/mL (“220 mg Naproxen”), the ratios of naproxen sodium to cholestyramine resin evaluated were in the range of 1:0 to 1:4 (naproxen sodium to cholestyramine resin), and the duration of the mixing reaction was 1 hr.

FIG. 9 Percentage of naproxen sodium, an NSAID, bound by cholestyramine resin, an IEX resin, in an aqueous solution following a mixing reaction with cholestyramine resin as a function of (i) the ratio of naproxen sodium to cholestyramine resin (mass to mass) in the aqueous solution, and (ii) the initial naproxen sodium concentration in the aqueous solution. In FIG. 9, the initial naproxen sodium concentrations in the aqueous solution evaluated were 22 mg/mL (“22 mg Naproxen”), 44 mg/mL (“44 mg Naproxen”), and 88 mg/mL (“88 mg Naproxen”), the ratios of naproxen sodium to cholestyramine resin evaluated were in the range of 1:1.25 to 1:2 (naproxen sodium to cholestyramine resin), and the duration of the mixing reaction was 0.5 hrs.

FIG. 10 Amount of naproxen sodium, an NSAID, remaining in an aqueous solution following a mixing reaction with cholestyramine resin, an IEX resin, as a function (i) the ratio of naproxen sodium to cholestyramine resin (mass to mass) in the aqueous solution, and (ii) the initial naproxen sodium concentration in the aqueous solution. In FIG. 10, the initial naproxen sodium concentrations in the aqueous solution evaluated were 22 mg/mL (“22 mg Naproxen”), 44 mg/mL (“44 mg Naproxen”), and 88 mg/mL (“88 mg Naproxen”), the ratios of naproxen sodium to cholestyramine resin evaluated were in the range of 1:1.25 to 1:2 (naproxen sodium to cholestyramine resin), and the duration of the mixing reaction was 0.5 hrs.

FIG. 11 pH of the aqueous solution of FIGS. 9-10 prior to the mixing reaction with cholestyramine resin, an IEX resin, as a function (i) the ratio of naproxen sodium to cholestyramine resin (mass to mass) in the aqueous solution, and (ii) the initial naproxen sodium concentration in the aqueous solution. In FIG. 11, the initial naproxen sodium concentrations in the aqueous solution evaluated were 22 mg/mL (“22 mg Naproxen”), 44 mg/mL (“44 mg Naproxen”), and 88 mg/mL (“88 mg Naproxen”), the ratios of naproxen sodium to cholestyramine resin evaluated were in the range of 1:1.25 to 1:2 (naproxen sodium to cholestyramine resin), and the duration of the mixing reaction was 0.5 hrs.

FIG. 12 Viscosity of various aqueous formulations following a mixing reaction between naproxen sodium, an NSAID, and cholestyramine resin, an IEX resin, as a function of xanthan gum and magnesium aluminometasilicate content in the aqueous formulation. In FIG. 12, the initial naproxen sodium concentration in the aqueous formulations was 2.20% (w/v), the ratio of naproxen sodium to cholestyramine resin in the aqueous formulations was 1:1.5 by mass (naproxen sodium to cholestyramine resin), and the amount of xanthan gum and magnesium aluminometasilicate ranged from 0.25-0.50% (w/v) and 0-0.25% (w/v), respectively.

FIG. 13 Percentage of naproxen sodium, an NSAID, bound by cholestyramine resin, an IEX resin, in an aqueous solution following a mixing reaction with cholestyramine resin as a function of the duration of the mixing reaction (“Time of Mixing”) in a 20 L scale-up batch. In FIG. 13, the initial naproxen sodium concentration in the aqueous formulations was 2.20% (w/v), the ratio of naproxen sodium to cholestyramine resin in the aqueous formulations was 1:1.5 by mass (naproxen sodium to cholestyramine resin).

FIG. 14 Amount of naproxen sodium, an NSAID, remaining in an aqueous solution following a mixing reaction with cholestyramine resin, an IEX resin, as a function of the duration of the mixing reaction (“Time of Mixing”) in a 20 L scale-up batch. In FIG. 14, the initial naproxen sodium concentration in the aqueous formulations was 2.20% (w/v), the ratio of naproxen sodium to cholestyramine resin in the aqueous formulations was 1:1.5 by mass (naproxen sodium to cholestyramine resin).

FIG. 15 Percentage of naproxen sodium, an NSAID, bound by cholestyramine resin, an IEX resin, in an aqueous solution following a mixing reaction with cholestyramine resin as a function of the duration of the mixing reaction (“Time of Mixing”) in a 20 L scale-up batch. In FIG. 15, the initial naproxen sodium concentration in the aqueous formulations was 2.20% (w/v), the ratio of naproxen sodium to cholestyramine resin in the aqueous formulations was 1:1.25 by mass (naproxen sodium to cholestyramine resin).

FIG. 16 Amount of naproxen sodium, an NSAID, remaining in an aqueous solution following a mixing reaction with cholestyramine resin, an IEX resin, as a function of the duration of the mixing reaction (“Time of Mixing”) in a 20 L scale-up batch. In FIG. 16, the initial naproxen sodium concentration in the aqueous formulations was 2.20% (w/v), the ratio of naproxen sodium to cholestyramine resin in the aqueous formulations was 1:1.25 by mass (naproxen sodium to cholestyramine resin).

FIG. 17 Percentage of naproxen sodium, an NSAID, bound by cholestyramine resin, an IEX resin, in an aqueous solution following a mixing reaction with cholestyramine resin as a function of the duration of the mixing reaction (“Time of Mixing”) in a 185 L scale-up batch, and sampled at different reactor portions, i.e., the top, middle, and bottom of the reactor. In FIG. 17, the initial naproxen sodium concentration in the aqueous formulations was 2.20% (w/v), the ratio of naproxen sodium to cholestyramine resin in the aqueous formulations was 1:1.5 by mass (naproxen sodium to cholestyramine resin).

FIG. 18 Amount of naproxen sodium, an NSAID, remaining in an aqueous solution following a mixing reaction with cholestyramine resin, an IEX resin, as a function of the duration of the mixing reaction (“Time of Mixing”) in a 185 L scale-up batch, and sampled at different reactor portions, i.e., the top, middle, and bottom of the reactor. In FIG. 18, the initial naproxen sodium concentration in the aqueous formulations was 2.20% (w/v), the ratio of naproxen sodium to cholestyramine resin in the aqueous formulations was 1:1.5 by mass (naproxen sodium to cholestyramine resin).

FIG. 19 Percentage of naproxen sodium, an NSAID, bound by cholestyramine resin, an IEX resin, in an aqueous solution following a mixing reaction with cholestyramine resin as a function of the duration of the mixing reaction (“Time of Mixing”) in a 185 L scale-up batch for in-house stability, and sampled at different reactor portions, i.e., the top, middle, and bottom of the reactor. In FIG. 19, the initial naproxen sodium concentration in the aqueous formulations was 2.20% (w/v), the ratio of naproxen sodium to cholestyramine resin in the aqueous formulations was 1:1.5 by mass (naproxen sodium to cholestyramine resin).

FIG. 20 Amount of naproxen sodium, an NSAID, remaining in an aqueous solution following a mixing reaction with cholestyramine resin, an IEX resin, as a function of the duration of the mixing reaction (“Time of Mixing”) in a 185 L scale-up batch for in-house stability, and sampled at different reactor portions, i.e., the top, middle, and bottom of the reactor. In FIG. 20, the initial naproxen sodium concentration in the aqueous formulations was 2.20% (w/v), the ratio of naproxen sodium to cholestyramine resin in the aqueous formulations was 1:1.5 by mass (naproxen sodium to cholestyramine resin).

FIG. 21 Release profiles of naproxen from the 185 L scale-up batch for in-house stability formulation of FIGS. 19-20 in water (with 0.04% SDS) at 50 RPM and 37° C. In FIG. 21, the percent release refers to the amount of naproxen sodium released into solution from cholestyramine resin-bound naproxen sodium.

FIG. 22 Release profile of an in situ API-resin binding batch (squares and hashed line) compared to a naproxen-resin complex API batch (circles and solid line) in 1.0 M NaCl at 50 RPM. The naproxen-resin complex API batch was prepared by isolating and drying naproxen-resin precipitant made using an initial naproxen sodium concentration in the aqueous formulations was 0.125% (w/w), a ratio of naproxen sodium to cholestyramine resin in the aqueous formulations was 1:1 by mass (naproxen sodium to cholestyramine resin), and a duration of the mixing reaction of 4 hrs. The in situ API-resin binding batch was the 185 L scale-up batch for in-house stability formulation of FIGS. 19-20.

FIG. 23 pH values of a naproxen sodium formulation, as described herein, resulting from pH testing of the formulation after storage under different conditions. In FIG. 23, the storage conditions evaluated were storage at 25° C. (at ambient relative humidity) for 9 months (square series), storage at 40° C. (at ambient relative humidity) for 6 months (circle series), and storage at 60° C. (at ambient relative humidity) for 6 weeks (diamond series).

FIG. 24 values of naproxen sodium as a percentage of the label claim for a naproxen sodium formulation, as described herein, after storage under different conditions. In FIG. 24, the storage conditions evaluated were storage at 25° C. (at ambient relative humidity) for 9 months (square series), storage at 40° C. (at ambient relative humidity) for 6 months (circle series), and storage at 60° C. (at ambient relative humidity) for 6 weeks (diamond series).

FIG. 25 values of methylparaben as a percentage of the label claim for a naproxen sodium formulation, as described herein, after storage under different conditions. In FIG. 25, the storage conditions evaluated were storage at 25° C. (at ambient relative humidity) for 9 months (square series), storage at 40° C. (at ambient relative humidity) for 6 months (circle series), and storage at 60° C. (at ambient relative humidity) for 6 weeks (diamond series).

FIG. 26 values of propylparaben as a percentage of the label claim for a naproxen sodium formulation, as described herein, after storage under different conditions. In FIG. 26, the storage conditions evaluated were storage at 25° C. (at ambient relative humidity) for 9 months (square series), storage at 40° C. (at ambient relative humidity) for 6 months (circle series), and storage at 60° C. (at ambient relative humidity) for 6 weeks (diamond series).

FIG. 27 viscosity values of a naproxen sodium formulation, as described herein, resulting from viscosity testing of the formulation after storage under different conditions. In FIG. 27, the storage conditions evaluated were storage at 25° C. (at ambient relative humidity) for 9 months (square series), storage at 40° C. (at ambient relative humidity) for 6 months (circle series), and storage at 60° C. (at ambient relative humidity) for 6 weeks (diamond series).

FIG. 28 dissolution profile of naproxen sodium from a naproxen sodium formulation, as described herein, after manufacture (square series), and storage at 25° C. (at ambient relative humidity) for 3 months (circle series), 6 months (triangle series), and 9 months (asterisk series).

FIG. 29 dissolution profile of naproxen sodium from a naproxen sodium formulation, as described herein, after manufacture (square series), and storage at 40° C. (at ambient relative humidity) for 1 month (circle series), 2 months (triangle series), 9 months (asterisk series), and 6 months (diamond series).

FIG. 30 dissolution profile of naproxen sodium from a naproxen sodium formulation, as described herein, after manufacture (square series), and storage at 60° C. (at ambient relative humidity) for 0.5 months (circle series), 1 month (triangle series), and 1.5 months (asterisk series).

### DETAILED DESCRIPTION

Some embodiments provide an ion-exchange (“IEX”) resin-bound nonsteroidal anti-inflammatory drug (“NSAID”) complex (“IEX-NSAID complex”) comprising at least one NSAID or salt thereof bound to an IEX resin.

In some embodiments, an NSAID, as described herein, may comprise any compound selected from the group consisting of aceclofenac, acemetacin, acetylsalicylic acid, celecoxib, dexibuprofen, dexketoprofen, diclofenac, diflunisal, etodolac, etoricoxib, fenoprofen, flurbiprofen, ibuprofen, indomethacin, ketoprofen, ketorolac, lornoxicam, loxoprofen, mefenamic acid, meloxicam, nabumetone, naproxen, nimesulide, oxaprozin, pelubiprofen, phenylbutazone, piroxicam, salsalate, sulindac, tenoxicam, tolfenamic acid, tolmetin, zaltoprofen, any salt of the foregoing, and any combination of the foregoing, as well as any other known NSAID not expressly listed that would be readily envisaged by one skilled the art.

In certain embodiments, a salt of an NSAID, as described herein, may comprise an alkali salt of an NSAID. As used herein, the term “alkali salt of an NSAID” refers to a lithium salt of an NSAID, a sodium salt of an NSAID, or a potassium salt of an NSAID.

In some embodiments, a salt of an NSAID, as described herein, may comprise an alkaline salt of an NSAID. As used herein, the term “alkaline salt of an NSAID” refers to a magnesium salt of an NSAID, a calcium salt of an NSAID, or a barium salt of an NSAID.

In certain embodiments, a salt of an NSAID, as described herein, may comprise a pharmaceutical cation salt of an NSAID. As used herein, the term “pharmaceutical cation salt of an NSAID” refers to an aluminum salt of an NSAID, an arginine salt of an NSAID, a benzathine salt of an NSAID, a chloroprocaine salt of an NSAID, a choline salt of an NSAID, a diethanolamine salt of an NSAID, an ethanolamine salt of an NSAID, an ethylenediamine salt of an NSAID, a histidine salt of an NSAID, a lysine salt of an NSAID, a meglumine salt of an NSAID, a procaine salt of an NSAID, a triethylamine salt of an NSAID, a tromethamine salt of an NSAID, or a zinc salt of an NSAID.

In some embodiments, a salt of an NSAID, as described herein, may comprise a quaternary ammonium salt of an NSAID. As used herein, the term “quaternary ammonium salt of an NSAID” refers a salt of an NSAID, wherein the salt component is a compound containing the general chemical structure represented in Formula I:

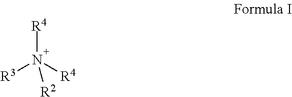

wherein:
R¹-R⁴ are each independently selected from the group consisting of hydrogen, an alkyl group, an alkenyl group, an alkynyl group, a cycloalkyl group, an aryl group, a heterocycle group, a halogen-substituted alkyl group, a halogen-substituted alkenyl group, a halogen-substituted alkynyl group, a halogen-substituted cycloalkyl group, a halogen-substituted aryl group, a halogen-substituted heterocycle group, an amino-functionalized alkyl group, an amino-functionalized alkenyl group, an amino-functionalized alkynyl group, an amino-functionalized cycloalkyl group, an amino-functionalized aryl group, an amino-functionalized heterocycle group, a hydroxy-functionalized alkyl group, a hydroxy-functionalized alkenyl group, a hydroxy-functionalized alkynyl group, a hydroxy-functionalized cycloalkyl group, a hydroxy-functionalized aryl group, a hydroxy-functionalized heterocycle group, a thio-functionalized alkyl group, a thio-functionalized alkenyl group, a thio-functionalized alkynyl group, a thio-functionalized cycloalkyl group, a thio-functionalized aryl group, a thio-functionalized heterocycle group, an amino acid-functionalized alkyl group, an amino acid-functionalized alkenyl group, an amino acid-functionalized alkynyl group, an amino acid-functionalized cycloalkyl group, an amino acid-functionalized aryl group, an amino acid-functionalized heterocycle group, an alkoxy group, an ether group, an ester group, a carboxylic acid group, and an amide group.

As used herein, the term “alkyl group” refers to any saturated aliphatic hydrocarbon, including straight chain and branched chain groups, that comprises between 1 and 20 carbon atoms total.

As used herein, the term “alkenyl group” refers to an unsaturated aliphatic hydrocarbon having at least one carbon-carbon double bond, including straight chain and branched chain groups, that comprises between 2 to 20 carbon atoms.

As used herein, the term “alkynyl group” refers to an unsaturated aliphatic hydrocarbon including at least one carbon-carbon triple bond, including straight chain and branched chain groups, that comprises between 2 to 20 carbon atoms.

As used herein, the term “cycloalkyl group” refers to a saturated on unsaturated all-carbon monocyclic or fused ring (i.e., rings which share an adjacent pair of carbon atoms) group wherein one of more of the rings does not have a completely conjugated pi-electron system.

As used herein, the term “aryl group” refers to an all-carbon monocyclic or fused-ring polycyclic (i.e., rings which share adjacent pairs of carbon atoms) having a completely conjugated pi-electron system.

As used herein, the term “heterocycle” refers to refers to a monocyclic or fused ring (i.e., rings which share an adjacent pair of atoms) having in the ring(s) one or more atoms, such as, for example, nitrogen, oxygen, and sulfur, that may or may not have a completely conjugated pi-electron system.

As used herein, the term “halogen-substituted” refers to an alkyl group, an alkenyl group, an alkynyl group, a cycloalkyl group, or an aryl group, a heterocycle, as described herein, that contains at least one fluorine, chlorine, bromine, or iodine atom.

As used herein, the term “amino-functionalized” refers to an alkyl group, an alkenyl group, an alkynyl group, a cycloalkyl group, or an aryl group, a heterocycle, as described herein, that contains at least one primary amine, secondary amine, or tertiary amine.

As used herein, the term “hydroxy-functionalized” refers to an alkyl group, an alkenyl group, an alkynyl group, a cycloalkyl group, or an aryl group, a heterocycle, as described herein, that contains at least one hydroxyl group.

As used herein, the term “thio-functionalized” refers to an alkyl group, an alkenyl group, an alkynyl group, a cycloalkyl group, or an aryl group, a heterocycle, as described herein, that contains at least one sulfur atom.

As used herein, the term “amino acid-functionalized” refers to an alkyl group, an alkenyl group, an alkynyl group, a cycloalkyl group, or an aryl group, a heterocycle, as described herein, that contains at least one primary amine, secondary amine, or tertiary amine, and at least one carboxylic acid.

As used herein, the term “alkoxy group” refers to any of an —O-alkyl, —O-alkenyl, —O— alkynyl, —O-cycloalkyl, —O-aryl, and —O-heterocycle, as defined herein.

In certain embodiments an IEX resin, as described herein, may comprise a functional group attached to a resin matrix.

In some embodiments, a functional group, as described herein, may comprise a Type I strong base anion, a Type II strong base anion, a weak base anion, an N-methylglucamine, or a combination thereof.

As used herein, the term “Type I strong base anion,” refers to a compound possessing the chemical structure represented in Formula II:

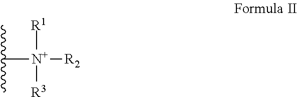

wherein:
R₁-R₃ are each independently selected from the group consisting of a methyl group, an ethyl group, an n-propyl group, an isopropyl, an n-butyl group, a sec-butyl group, a tert-butyl group, and an isobutyl group.

As used herein, the term “Type II strong base anion,” refers to a compound possessing the chemical structure represented in Formula III:

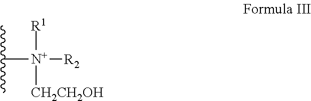

wherein:
R₁-R₂ are each independently selected from the group consisting of a methyl group, an ethyl group, an n-propyl group, an isopropyl, an n-butyl group, a sec-butyl group, a tert-butyl group, and an isobutyl group.

As used herein, the term “weak strong base anion,” refers to a compound possessing the chemical structure represented in Formula IV:

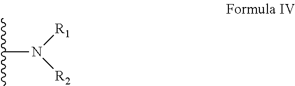

wherein:
R₁ is selected from the group consisting of a hydrogen, a methyl group, an ethyl group, an n-propyl group, an isopropyl, an n-butyl group, a sec-butyl group, a tert-butyl group, and an isobutyl group; and
R₂ is selected from the group consisting of a methyl group, an ethyl group, an n-propyl group, an isopropyl, an n-butyl group, a sec-butyl group, a tert-butyl group, and an isobutyl group.

As used herein, the term “N-methylglucamine” refers to a compound possessing the chemical structure represented in Formula V:

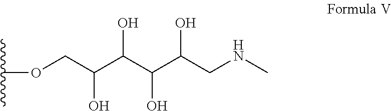 [SMILES: *OCC(O)C(O)C(O)C(O)CNC]

In some embodiments, a resin matrix, as described herein, may comprise a polymer selected from the group consisting of polyacrylate, polymethacrylate, and polystyrene.

In certain embodiments, a resin matrix, as described herein, may comprise a copolymer comprising at least two different monomer units selected from the group consisting of acrylate, divinylbenzene, methacrylate, and styrene.

In some embodiments, an IEX resin, as described herein, may comprise an amine-functionalized polymer. As used herein, the term “amine-functionalized polymer” refers to a polymer selected from the group consisting of poly(4-aminostyrene), poly(4-vinylpyridine), polyallylamine, polyethyleneimine, poly-L-lysine, polyvinylamine, and any quaternized derivative thereof.

In certain embodiments, an IEX resin, as described herein, may comprise cholestyramine, colestipol, colesevelam, and any combination thereof.

In some embodiments, an IEX resin, as described herein, may comprise a commercially-available IEX resin selected from the group consisting of Duolite™ AP143/1073 Resin, Duolite™ AP143/1083 Resin, Purolite® A430MR Resin, Reillex™ 402 Ion-exchange Resin, and Seplife® LPF851 Powder Resin.

In some embodiments, an IEX resin, as described herein, may comprise an average particle size. In certain embodiments, an average particle size, as described herein, may be less than about 1500 μm. In some embodiments, an average particle size, as described herein, may be less than about 1000 μm. In certain embodiments, an average particle size, as described herein, may be less than about 500 μm. In some embodiments, an average particle size, as described herein, may be less than about 150 μm.

In certain embodiments, an IEX-NSAID complex, as described herein, may comprise between about 38% NSAID or salt thereof by weight and about 60% NSAID or salt thereof by weight. In some embodiments, an IEX-NSAID complex, as described herein, may comprise between about 42% NSAID or salt thereof by weight and about 56% NSAID or salt thereof by weight. In certain embodiments, an IEX-NSAID complex, as described herein, may comprise between about 46% NSAID or salt thereof by weight and about 52% NSAID or salt thereof by weight.

In some embodiments, an IEX-NSAID complex, as described herein, may release at least about 50% of the bound NSAID or salt thereof within 10 minutes during a dissolution test. In some embodiments, an IEX-NSAID complex, as described herein, may release at least about 50% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an IEX-NSAID complex, as described herein, may release at least about 60% of the bound NSAID or salt thereof within 10 minutes during a dissolution test. In some embodiments, an IEX-NSAID complex, as described herein, may release at least about 60% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an IEX-NSAID complex, as described herein, may release at least about 70% of the bound NSAID or salt thereof within 10 minutes during a dissolution test. In some embodiments, an IEX-NSAID complex, as described herein, may release at least about 70% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an IEX-NSAID complex, as described herein, may release at least about 80% of the bound NSAID or salt thereof within 10 minutes during a dissolution test. In some embodiments, an IEX-NSAID complex, as described herein, may release at least about 80% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an IEX-NSAID complex, as described herein, may release at least about 75% of the bound NSAID or salt thereof within 30 minutes during a dissolution test. In some embodiments, an IEX-NSAID complex, as described herein, may release at least about 75% of the bound NSAID or salt thereof within 30 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an IEX-NSAID complex, as described herein, may release at least about 80% of the bound NSAID or salt thereof, as described herein, within 30 minutes during a dissolution test. In some embodiments, an IEX-NSAID complex, as described herein, may release at least about 80% of the bound NSAID or salt thereof within 30 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an IEX-NSAID complex, as described herein, may release at least about 85% of the bound NSAID or salt thereof within 30 minutes during a dissolution test. In some embodiments, an IEX-NSAID complex, as described herein, may release at least about 85% of the bound NSAID or salt thereof within 30 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an IEX-NSAID complex, as described herein, may release at least about 80% of the bound NSAID or salt thereof within 60 minutes during a dissolution test. In some embodiments, an IEX-NSAID complex, as described herein, may release at least about 80% of the bound NSAID or salt thereof within 60 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an IEX-NSAID complex, as described herein, may release at least about 85% of the bound NSAID or salt thereof within 60 minutes during a dissolution test. In some embodiments, an IEX-NSAID complex, as described herein, may release at least about 85% of the bound NSAID or salt thereof within 60 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an IEX-NSAID complex, as described herein, may release at least about 90% of the bound NSAID or salt thereof within 60 minutes during a dissolution test. In some embodiments, an IEX-NSAID complex, as described herein, may release at least about 90% of the bound NSAID or salt thereof within 60 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an amount of an IEX-NSAID complex, as described herein, may be provided in a liquid dosage form. In certain embodiments, a liquid dosage form, as described herein, may be adapted to be orally administered to a subject. Liquid dosage forms falling within the scope of disclosure contemplated herein include, but are not limited to aqueous dispersions, oil-based dispersions, aqueous suspensions, oil-based suspensions, aqueous solutions, oil-based solutions, beverages, syrups, elixirs, emulsions, and the like. In some embodiments, a liquid dosage form, as described herein, may be an aqueous dispersion, an aqueous suspension, or an aqueous solution. In certain embodiments, a liquid dosage form, as described herein, may be an aqueous suspension.

In certain embodiments, a liquid dosage form, as described herein, may comprise an amount of NSAID or salt thereof in an IEX-NSAID complex, as described herein, that is about 1.0% wt./vol. to about 5.0% wt./vol. (relative to the volume of the liquid dosage form). In some embodiments, a liquid dosage form, as described herein, may comprise an amount of NSAID or salt thereof in an IEX-NSAID complex, as described herein, that is about 1.5% wt./vol. to about 4.0% wt./vol. (relative to the volume of the liquid dosage form). In certain embodiments, a liquid dosage form, as described herein, may comprise an amount of NSAID or salt thereof in an IEX-NSAID complex, as described herein, that is about 2.0% wt./vol. to about 3.0% wt./vol. (relative to the volume of the liquid dosage form). In some embodiments, a liquid dosage form, as described herein, may comprise an amount of NSAID or salt thereof in an IEX-NSAID complex, as described herein, that is about 2.0% wt./vol. to about 2.5% wt./vol. (relative to the volume of the liquid dosage form).

In certain embodiments, a liquid dosage form, as described herein, may comprise an amount of one more or more sweeteners. As used herein, the term “sweetener,” refers to any compound that increases the sweetness of a liquid dosage form, as described herein, when included therein. Sweeteners falling within the scope of disclosure contemplated herein include, but are not limited to acesulfame potassium, advantame, arabinose, aspartame, cyclamate, dextrin, dextrose, disaccharide, erythritol, fructose, fucose, galactose, glucose, isomalt, lactitol, lactose, maltitol, maltodextrin, maltose, mannitol, mannose, monkfruit extract, neotame, rhamnose, ribose, saccharin, saccharose, sorbitol, steviol glycosides, sucrose, sucralose, thaumatin, trehalose, xylitol, and xylose, as well as any other known sweetener not expressly listed that would be readily envisaged by one skilled the art.

In some embodiments, a liquid dosage form, as described herein, may comprise an amount of xylitol, an amount of sucralose, or the combination thereof.

In certain embodiments, a liquid dosage form, as described herein, may comprise an amount of xylitol that is about 5% wt./vol. to about 40% wt./vol. (relative to the volume of the liquid dosage form), such as, for example, 5% wt./vol., 7.5% wt./vol., 10% wt./vol., 12.5% wt./vol., 15% wt./vol., 17.5% wt./vol., 20% wt./vol., 22.5% wt./vol., 25% wt./vol., 27.5% wt./vol., 30% wt./vol., 32.5% wt./vol., 35% wt./vol., 37.5% wt./vol., 40% wt./vol., or any value in between any two of the preceding amounts.

In some embodiments, a liquid dosage form, as described herein, may comprise an amount of sucralose that is about 0.025% wt./vol. to about 1.0% wt./vol. (relative to the volume of the liquid dosage form), such as, for example, 0.025% wt./vol., 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., 0.625% wt./vol., 0.65% wt./vol., 0.675% wt./vol., 0.70% wt./vol., 0.725% wt./vol., 0.75% wt./vol., 0.775% wt./vol., 0.80% wt./vol., 0.825% wt./vol., 0.85% wt./vol., 0.875% wt./vol., 0.90% wt./vol., 0.925% wt./vol., 0.95% wt./vol., 0.975% wt./vol., 1.0% wt./vol., or any value in between any two of the preceding amounts.

In some embodiments, a liquid dosage form, as described herein, may comprise an amount of one more or more thickeners. As used herein, the term “thickener,” refers to any compound that increases the viscosity of a liquid dosage form, as described herein, when included therein. Thickeners falling within the scope of disclosure contemplated herein include, but are not limited to acacia, agar, alginates, alginic acid, alumina, aluminum monostearate, amidated pectins, bentonite, carbomers, carboxymethylcellulose, carrageenan, caseinates, cellulose, chitosan, dextran, dextrin, ethylcellulose, ethylene vinyl acetates, fatty acids, gelatin, gellan gum, glycerin, guar gum, hectorite, hydroxyethylcellulose, hydroxymethylcellulose, hydroxypropylcellulose, hydroxypropyl methylcellulose, karaya gum, locust bean, magnesia, magnesium aluminometasilicates, such as Neusilin®, magnesium aluminum silicates, methylcellulose, microcrystalline cellulose, pectin, polyethylene glycol, polyethylene oxide, propylene glycol, propylene glycol alginate, polyvinyl alcohol, polyvinylpyrrolidones, pullulan, silica, silicones, starches, tragacanth, xanthan gum, any salt of the foregoing, and any hydrate of the foregoing, as well as other known thickeners not expressly listed that would be readily envisaged by one skilled the art.

In certain embodiments, a liquid dosage form, as described herein, may comprise an amount of glycerin, an amount of xanthan gum, an amount of a magnesium aluminometasilicate or a hydrate thereof, or any combination thereof.

In some embodiments, a liquid dosage form, as described herein, may comprise an amount of glycerin that is about 5% wt./vol. to about 50% wt./vol. (relative to the volume of the liquid dosage form), such as, for example, 5.0% wt./vol., 7.5% wt./vol., 10.0% wt./vol., 12.5% wt./vol., 15.0% wt./vol., 17.5% wt./vol., 20.0% wt./vol., 22.5% wt./vol., 25.0% wt./vol., 27.5% wt./vol., 30.0% wt./vol., 32.5% wt./vol., 35.0% wt./vol., 37.5% wt./vol., 40.0% wt./vol., 42.5% wt./vol., 45.0% wt./vol., 47.5% wt./vol., 50.0% wt./vol., or any value in between any two of the preceding amounts.

In some embodiments, a liquid dosage form, as described herein, may comprise an amount of xanthan gum that is about 0.05% wt./vol. to about 1.0% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., 0.625% wt./vol., 0.65% wt./vol., 0.675% wt./vol., 0.70% wt./vol., 0.725% wt./vol., 0.75% wt./vol., 0.775% wt./vol., 0.80% wt./vol., 0.825% wt./vol., 0.85% wt./vol., 0.875% wt./vol., 0.90% wt./vol., 0.925% wt./vol., 0.95% wt./vol., 0.975% wt./vol., 1.0% wt./vol., or any value in between any two of the preceding amounts.

In some embodiments, a liquid dosage form, as described herein, may comprise an amount of a magnesium aluminometasilicate or hydrate thereof, that is about 0.05% wt./vol. to about 0.50% wt./vol. (relative to the volume of the liquid dosage form), such as for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In some embodiments, a liquid dosage form, as described herein, may comprise one or more of: (i) an amount of glycerin that is about 5% wt./vol. to about 50% wt./vol. (relative to the volume of the liquid dosage form), such as, for example, 5.0% wt./vol., 7.5% wt./vol., 10.0% wt./vol., 12.5% wt./vol., 15.0% wt./vol., 17.5% wt./vol., 20.0% wt./vol., 22.5% wt./vol., 25.0% wt./vol., 27.5% wt./vol., 30.0% wt./vol., 32.5% wt./vol., 35.0% wt./vol., 37.5% wt./vol., 40.0% wt./vol., 42.5% wt./vol., 45.0% wt./vol., 47.5% wt./vol., 50.0% wt./vol., or any value in between any two of the preceding amounts; (ii) an amount of xanthan gum that is about 0.05% wt./vol. to about 1.0% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., 0.625% wt./vol., 0.65% wt./vol., 0.675% wt./vol., 0.70% wt./vol., 0.725% wt./vol., 0.75% wt./vol., 0.775% wt./vol., 0.80% wt./vol., 0.825% wt./vol., 0.85% wt./vol., 0.875% wt./vol., 0.90% wt./vol., 0.925% wt./vol., 0.95% wt./vol., 0.975% wt./vol., 1.0% wt./vol., or any value in between any two of the preceding amounts; and (iii) an amount of a magnesium aluminometasilicate or hydrate thereof, that is about 0.05% wt./vol. to about 0.50% wt./vol. (relative to the volume of the liquid dosage form), such as for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In some embodiments, a liquid dosage form, as described herein, may comprise an amount of one more or more preservatives. As used herein, the term “preservative,” refers to any compound that increases the improves the shelf life of a liquid dosage form, as described herein, when included therein. Preservatives falling within the scope of disclosure contemplated herein include, but are not limited to alkali-/alkaline-metal benzoates (such as sodium benzoate, potassium benzoate, magnesium benzoate, calcium benzoate, and the like), alkali-/alkaline-metal metabisulfites (such as sodium metabisulfite, potassium metabisulfite, magnesium metabisulfite, calcium metabisulfite, and the like), alkali-/alkaline-metal sorbates (such as sodium sorbate, potassium sorbate, magnesium sorbate, calcium sorbate, and the like), benzoic acid, edetate sodium, hydroxyparabenzoate, parabens (such as methylparaben, ethylparaben, propylparaben, isopropylparaben, butylparaben, isobutylparaben, pentylparaben, benzylparaben, and the like), polysorbate 20, polysorbate 40, polysorbate 60, polysorbate 80, propylene glycol, propylene glycol diacetate, propylene glycol dicaprylate, propylene glycol monostearate, as well as other known preservatives not expressly listed that would be readily envisaged by one skilled the art.

In certain embodiments, a liquid dosage form, as described herein, may comprise an amount of edetate sodium, methylparaben, polysorbate 80, potassium sorbate, propylene glycol, propylparaben, sodium benzoate, or any combination of the foregoing.

In some embodiments, a liquid dosage form, as described herein, may comprise an amount of edetate sodium that is about 0.05% wt./vol. to about 0.25% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., or any value in between any two of the preceding amounts.

In certain embodiments, a liquid dosage form, as described herein, may comprise an amount of methylparaben that is about 0.05% wt./vol. to about 0.50% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In some embodiments, a liquid dosage form, as described herein, may comprise an amount of polysorbate 80 that is about 0.10% wt./vol. to about 0.60% wt./vol. (relative to the volume of the liquid dosage form), such as, for example, 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., or any value in between any two of the preceding amounts.

In certain embodiments, a liquid dosage form, as described herein, may comprise an amount of potassium sorbate that is about 0.05% wt./vol. to about 0.50% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In some embodiments, a liquid dosage form, as described herein, may comprise an amount of propylene glycol that is about 0.50% wt./vol. to about 10.0% wt./vol. (relative to the volume of the liquid dosage form), such as, for example, 0.50% wt./vol., 0.75% wt./vol., 1.0% wt./vol., 1.25% wt./vol., 1.5% wt./vol., 1.75% wt./vol., 2.0% wt./vol., 2.25% wt./vol., 2.5% wt./vol., 2.75% wt./vol., 3.0% wt./vol., 3.25% wt./vol., 3.5% wt./vol., 3.75% wt./vol., 4.0% wt./vol., 4.25% wt./vol., 4.5% wt./vol., 4.75% wt./vol., 5.0% wt./vol., 5.25% wt./vol., 5.5% wt./vol., 5.75% wt./vol., 6.0% wt./vol., 6.25% wt./vol., 6.5% wt./vol., 6.75% wt./vol., 7.0% wt./vol., 7.25% wt./vol., 7.5% wt./vol., 7.75% wt./vol., 8.0% wt./vol., 8.25% wt./vol., 8.5% wt./vol., 8.75% wt./vol., 9.0% wt./vol., 9.25% wt./vol., 9.5% wt./vol., 9.75% wt./vol., 10.0% wt./vol., or any value in between any two of the preceding amounts.

In certain embodiments, a liquid dosage form, as described herein, may comprise an amount of propylparaben that is about 0.01% wt./vol. to about 0.30% wt./vol. (relative to the volume of the liquid dosage form), such as, for example, 0.01% wt./vol., 0.025% wt./vol., 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., or any value in between any two of the preceding amounts.

In some embodiments, a liquid dosage form, as described herein, may comprise an amount of sodium benzoate that is about 0.10% wt./vol. to about 0.50% wt./vol. (relative to the volume of the liquid dosage form), such as, for example, 0.10% wt./vol., 0.12% wt./vol., 0.14% wt./vol., 0.16% wt./vol., 0.18% wt./vol., 0.20% wt./vol., 0.22% wt./vol., 0.24% wt./vol., 0.26% wt./vol., 0.28% wt./vol., 0.30% wt./vol., 0.32% wt./vol., 0.34% wt./vol., 0.36% wt./vol., 0.38% wt./vol., 0.40% wt./vol., 0.42% wt./vol., 0.44% wt./vol., 0.46% wt./vol., 0.48% wt./vol., 0.50% wt./vol., or any value in between any two of the preceding amounts.

In certain embodiments, a liquid dosage form, as described herein, may comprise one or more of: (i) an amount of edetate sodium that is about 0.05% wt./vol. to about 0.25% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., or any value in between any two of the preceding amounts; (ii) an amount of methylparaben that is about 0.05% wt./vol. to about 0.50% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts; (iii) an amount of polysorbate 80 that is about 0.10% wt./vol. to about 0.60% wt./vol. (relative to the volume of the liquid dosage form), such as, for example, 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., or any value in between any two of the preceding amounts; (iv) an amount of potassium sorbate that is about 0.05% wt./vol. to about 0.50% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts; (v) an amount of propylene glycol that is about 0.50% wt./vol. to about 10.0% wt./vol. (relative to the volume of the liquid dosage form), such as, for example, 0.50% wt./vol., 0.75% wt./vol., 1.0% wt./vol., 1.25% wt./vol., 1.5% wt./vol., 1.75% wt./vol., 2.0% wt./vol., 2.25% wt./vol., 2.5% wt./vol., 2.75% wt./vol., 3.0% wt./vol., 3.25% wt./vol., 3.5% wt./vol., 3.75% wt./vol., 4.0% wt./vol., 4.25% wt./vol., 4.5% wt./vol., 4.75% wt./vol., 5.0% wt./vol., 5.25% wt./vol., 5.5% wt./vol., 5.75% wt./vol., 6.0% wt./vol., 6.25% wt./vol., 6.5% wt./vol., 6.75% wt./vol., 7.0% wt./vol., 7.25% wt./vol., 7.5% wt./vol., 7.75% wt./vol., 8.0% wt./vol., 8.25% wt./vol., 8.5% wt./vol., 8.75% wt./vol., 9.0% wt./vol., 9.25% wt./vol., 9.5% wt./vol., 9.75% wt./vol., 10.0% wt./vol., or any value in between any two of the preceding amounts; (vi) an amount of propylparaben that is about 0.01% wt./vol. to about 0.30% wt./vol. (relative to the volume of the liquid dosage form), such as, for example, 0.01% wt./vol., 0.025% wt./vol., 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., or any value in between any two of the preceding amounts; and (vii) an amount of sodium benzoate that is about 0.10% wt./vol. to about 0.50% wt./vol. (relative to the volume of the liquid dosage form), such as, for example, 0.10% wt./vol., 0.12% wt./vol., 0.14% wt./vol., 0.16% wt./vol., 0.18% wt./vol., 0.20% wt./vol., 0.22% wt./vol., 0.24% wt./vol., 0.26% wt./vol., 0.28% wt./vol., 0.30% wt./vol., 0.32% wt./vol., 0.34% wt./vol., 0.36% wt./vol., 0.38% wt./vol., 0.40% wt./vol., 0.42% wt./vol., 0.44% wt./vol., 0.46% wt./vol., 0.48% wt./vol., 0.50% wt./vol., or any value in between any two of the preceding amounts.

In certain embodiments, a liquid dosage form, as described herein, may comprise an amount of one more or more flavorants. As used herein, the term “flavorant,” refers to any compound that imparts a flavor to a liquid dosage form, as described herein, when included therein. Flavorants falling within the scope of disclosure contemplated herein include, but are not limited to apple flavoring, apricot flavoring, banana flavoring, berry flavoring, blackberry flavoring, blueberry flavoring, bubble gum flavoring, cherry flavoring, chocolate flavoring, cinnamon flavoring, citrus flavoring, coconut flavoring, cotton candy flavoring, cranberry flavoring, fruit punch flavoring, grape flavoring, grapefruit flavoring, kiwi flavoring, lemon flavoring, lime flavoring, mango flavoring, mint flavoring, orange flavoring, peach flavoring, pineapple flavoring, plum flavoring, pomegranate flavoring, raspberry flavoring, strawberry flavoring, tangerine flavoring, vanilla flavoring, and watermelon flavoring.

In some embodiments, a liquid dosage form, as described herein, may comprise an amount of flavorant that is about 0.10% wt./vol. to about 3.0% wt./vol. (relative to the volume of the liquid dosage form), such as, for example, 0.10% wt./vol., 0.15% wt./vol., 0.20% wt./vol., 0.25% wt./vol., 0.30% wt./vol., 0.35% wt./vol., 0.40% wt./vol., 0.45% wt./vol., 0.50% wt./vol., 0.55% wt./vol., 0.60% wt./vol., 0.65% wt./vol., 0.70% wt./vol., 0.75% wt./vol., 0.80% wt./vol., 0.85% wt./vol., 0.90% wt./vol., 0.95% wt./vol., 1.0% wt./vol., 1.05% wt./vol., 1.10% wt./vol., 1.15% wt./vol., 1.20% wt./vol., 1.25% wt./vol., 1.30% wt./vol., 1.35% wt./vol., 1.40% wt./vol., 1.45% wt./vol., 1.50% wt./vol., 1.55% wt./vol., 1.60% wt./vol., 1.65% wt./vol., 1.70% wt./vol., 1.75% wt./vol., 1.80% wt./vol., 1.85% wt./vol., 1.90% wt./vol., 1.95% wt./vol., 2.0% wt./vol., 2.05% wt./vol., 2.10% wt./vol., 2.15% wt./vol., 2.20% wt./vol., 2.25% wt./vol., 2.30% wt./vol., 2.35% wt./vol., 2.40% wt./vol., 2.45% wt./vol., 2.50% wt./vol., 2.55% wt./vol., 2.60% wt./vol., 2.65% wt./vol., 2.70% wt./vol., 2.75% wt./vol., 2.80% wt./vol., 2.85% wt./vol., 2.90% wt./vol., 2.95% wt./vol., 3.0% wt./vol., or any value in between any two of the preceding amounts.

In some embodiments, a liquid dosage form, as described herein, may comprise an amount of grape flavoring.

In certain embodiments, a liquid dosage form, as described herein, may comprise an amount of grape flavoring that is about 0.10% wt./vol. to about 3.0% wt./vol. (relative to the volume of the liquid dosage form), such as, for example, 0.10% wt./vol., 0.15% wt./vol., 0.20% wt./vol., 0.25% wt./vol., 0.30% wt./vol., 0.35% wt./vol., 0.40% wt./vol., 0.45% wt./vol., 0.50% wt./vol., 0.55% wt./vol., 0.60% wt./vol., 0.65% wt./vol., 0.70% wt./vol., 0.75% wt./vol., 0.80% wt./vol., 0.85% wt./vol., 0.90% wt./vol., 0.95% wt./vol., 1.0% wt./vol., 1.05% wt./vol., 1.10% wt./vol., 1.15% wt./vol., 1.20% wt./vol., 1.25% wt./vol., 1.30% wt./vol., 1.35% wt./vol., 1.40% wt./vol., 1.45% wt./vol., 1.50% wt./vol., 1.55% wt./vol., 1.60% wt./vol., 1.65% wt./vol., 1.70% wt./vol., 1.75% wt./vol., 1.80% wt./vol., 1.85% wt./vol., 1.90% wt./vol., 1.95% wt./vol., 2.0% wt./vol., 2.05% wt./vol., 2.10% wt./vol., 2.15% wt./vol., 2.20% wt./vol., 2.25% wt./vol., 2.30% wt./vol., 2.35% wt./vol., 2.40% wt./vol., 2.45% wt./vol., 2.50% wt./vol., 2.55% wt./vol., 2.60% wt./vol., 2.65% wt./vol., 2.70% wt./vol., 2.75% wt./vol., 2.80% wt./vol., 2.85% wt./vol., 2.90% wt./vol., 2.95% wt./vol., 3.0% wt./vol., or any value in between any two of the preceding amounts.

In some embodiments, a liquid dosage form, as described herein, may comprise a density. In certain embodiments a density of a liquid dosage form, as described herein, may be about 0.90 g/mL to about 1.30 g/mL, such as, for example 0.90 g/mL, 0.91 g/mL, 0.92 g/mL, 0.93 g/mL, 0.94 g/mL, 0.95 g/mL, 0.96 g/mL, 0.97 g/mL, 0.98 g/mL, 0.99 g/mL, 1.0 g/mL, 1.01 g/mL, 1.02 g/mL, 1.03 g/mL, 1.04 g/mL, 1.05 g/mL, 1.06 g/mL, 1.07 g/mL, 1.08 g/mL, 1.09 g/mL, 1.10 g/mL, 1.11 g/mL, 1.12 g/mL, 1.13 g/mL, 1.14 g/mL, 1.15 g/mL, 1.16 g/mL, 1.17 g/mL, 1.18 g/mL, 1.19 g/mL, 1.20 g/mL, 1.21 g/mL, 1.22 g/mL, 1.23 g/mL, 1.24 g/mL, 1.25 g/mL, 1.26 g/mL, 1.27 g/mL, 1.28 g/mL, 1.29 g/mL, 1.30 g/mL, or any value in between any two of the preceding amounts.

In certain embodiments, a liquid dosage form, as described herein, may comprise a viscosity. In some embodiments a viscosity of a liquid dosage form, as described herein, may be about 10 cP to about 1000 cP. In certain embodiments, a viscosity of a liquid dosage form, as described herein, may be about 50 cP to about 800 cP. In some embodiments, a viscosity of a liquid dosage form, as described herein, may be about 100 cP to about 700 cP. In certain embodiments, a viscosity of a liquid dosage form, as described herein, may be about 300 cP to about 700 cP. In some embodiments, a viscosity of a liquid dosage form, as described herein, may be about 300 cP to about 700 cP after storage at 25° C. for at least 0.5 months, at least 1 months, at least 3 months, at least 6 months, at least 9 months, at least 12 months, or any combination thereof. In certain embodiments, a viscosity of a liquid dosage form, as described herein, may be about 300 cP to about 700 cP after storage at 40° C. for at least 0.5 months, at least 1 months, at least 3 months, at least 6 months, or any combination thereof. In some embodiments, a viscosity of a liquid dosage form, as described herein, may be about 300 cP to about 700 cP after storage at 60° C. for at least 0.5 months, at least 1 month, at least 1.5 months, or any combination thereof.

In some embodiments, a liquid dosage form, as described herein, may comprise a dispersibility. In certain embodiments a dispersibility of a liquid dosage form, as described herein, may be at least about 80%. In some embodiments, a dispersibility of a liquid dosage form, as described herein, may be at least about 85%. In certain embodiments, a dispersibility of a liquid dosage form, as described herein, may be at least about 90%. In some embodiments, a dispersibility of a liquid dosage form, as described herein, may be at least about 95%.

In some embodiments, a liquid dosage form, as described herein, may pass a microbial test selected from USP <51> (14 day interval), USP <51> (28 day interval), USP <60>, USP <61>, USP <62>, and any combination thereof, after storage at 25° C. for at least 0.5 months, at least 1 months, at least 3 months, at least 6 months, at least 9 months, at least 12 months, or any combination thereof. In certain embodiments, a liquid dosage form, as described herein, may pass a microbial test selected from USP <51> (14 day interval), USP <51> (28 day interval), USP <60>, USP <61>, USP <62>, and any combination thereof, after storage at 40° C. for at least 0.5 months, at least 1 months, at least 3 months, at least 6 months, or any combination thereof. In certain embodiments, a liquid dosage form, as described herein, may pass a microbial test selected from USP <51> (14 day interval), USP <51> (28 day interval), USP <60>, USP <61>, USP <62>, and any combination thereof, after storage at 60° C. for at least 0.5 months, at least 1 month, at least 1.5 months, or any combination thereof.

In some embodiments, a liquid dosage form, as described herein, after storage at 25° C. for at least 0.5 months, at least 1 months, at least 3 months, at least 6 months, at least 9 months, at least 12 months, or any combination thereof, may satisfy at least one of: (i) no more than about 0.2% of Naproxen Related Compound A (“NRC A”) contained therein; (ii) no more than about 0.2% of Naproxen Related Compound K (“NRC K”) contained therein; (iii) no more than about 0.2% of Naproxen Related Compound L (“NRC L”) contained therein; (iv) no more than about 0.2% of any Individual Unspecified Impurities (“IUI”) contained therein; (v) no more than about 1.5% of Total Impurities contained therein; and (vi) any combination thereof.

In some embodiments, a liquid dosage form, as described herein, after storage at 25° C. for at least 0.5 months, at least 1 months, at least 3 months, at least 6 months, at least 9 months, at least 12 months, or any combination thereof, may satisfy at least one of: (i) no more than about 0.1% of NRC A contained therein; (ii) no more than about 0.1% of NRC K contained therein; (iii) no more than about 0.1% of NRC L contained therein; (iv) no more than about 0.1% of any IUI contained therein; (v) no more than about 0.5% of Total Impurities contained therein; and (vi) any combination thereof.

In some embodiments, a liquid dosage form, as described herein, after storage at 25° C. for at least 0.5 months, at least 1 months, at least 3 months, at least 6 months, at least 9 months, at least 12 months, or any combination thereof, may satisfy at least one of: (i) no more than about 0.05% of NRC A contained therein; (ii) no more than about 0.05% of NRC K contained therein; (iii) no more than about 0.05% of NRC L contained therein; (iv) no more than about 0.05% of any IUI contained therein; (v) no more than about 0.1% of Total Impurities contained therein; and (vi) any combination thereof.

In some embodiments, a liquid dosage form, as described herein, after storage at 40° C. for at least 0.5 months, at least 1 months, at least 3 months, at least 6 months, or any combination thereof, may satisfy at least one of: (i) no more than about 0.2% of NRC A contained therein; (ii) no more than about 0.2% of NRC K contained therein; (iii) no more than about 0.2% of NRC L contained therein; (iv) no more than about 0.2% of any IUI contained therein; (v) no more than about 1.5% of Total Impurities contained therein; and (vi) any combination thereof.

In some embodiments, a liquid dosage form, as described herein, after storage at 40° C. for at least 0.5 months, at least 1 months, at least 3 months, at least 6 months, or any combination thereof, may satisfy at least one of: (i) no more than about 0.1% of NRC A contained therein; (ii) no more than about 0.1% of NRC K contained therein; (iii) no more than about 0.1% of NRC L contained therein; (iv) no more than about 0.1% of any IUI contained therein; (v) no more than about 0.5% of Total Impurities contained therein; and (vi) any combination thereof.

In some embodiments, a liquid dosage form, as described herein, after storage at 40° C. for at least 0.5 months, at least 1 months, at least 3 months, at least 6 months, or any combination thereof, may satisfy at least one of: (i) no more than about 0.05% of NRC A contained therein; (ii) no more than about 0.05% of NRC K contained therein; (iii) no more than about 0.05% of NRC L contained therein; (iv) no more than about 0.05% of any IUI contained therein; (v) no more than about 0.1% of Total Impurities contained therein; and (vi) any combination thereof.

In some embodiments, a liquid dosage form, as described herein, after storage at 60° C. for at least 0.5 months, at least 1 month, at least 1.5 months, or any combination thereof, may satisfy at least one of: (i) no more than about 0.2% of NRC A contained therein; (ii) no more than about 0.2% of NRC K contained therein; (iii) no more than about 0.2% of NRC L contained therein; (iv) no more than about 0.2% of any IUI contained therein; (v) no more than about 1.5% of Total Impurities contained therein; and (vi) any combination thereof.

In some embodiments, a liquid dosage form, as described herein, after storage at 60° C. for at least 0.5 months, at least 1 month, at least 1.5 months, or any combination thereof, may satisfy at least one of: (i) no more than about 0.1% of NRC A contained therein; (ii) no more than about 0.1% of NRC K contained therein; (iii) no more than about 0.1% of NRC L contained therein; (iv) no more than about 0.15% of any IUI contained therein; (v) no more than about 0.5% of Total Impurities contained therein; and (vi) any combination thereof.

In some embodiments, a liquid dosage form, as described herein, after storage at 60° C. for at least 0.5 months, at least 1 month, at least 1.5 months, or any combination thereof, may satisfy at least one of: (i) no more than about 0.05% of NRC A contained therein; (ii) no more than about 0.05% of NRC K contained therein; (iii) no more than about 0.05% of NRC L contained therein; (iv) no more than about 0.1% of any IUI contained therein; (v) no more than about 0.1% of Total Impurities contained therein; and (vi) any combination thereof.

In some embodiments, a liquid dosage form, as described herein, may comprise a pH of between about pH 6.0 and about pH 8.0, such as for example pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, or pH 8.0. In certain embodiments, a liquid dosage form, as described herein, may comprise a pH of between about pH 6.0 and about pH 8.0, as described herein, after storage at 25° C. for at least 0.5 months, at least 1 months, at least 3 months, at least 6 months, at least 9 months, at least 12 months, or any combination thereof. In some embodiments, a liquid dosage form, as described herein, may comprise a pH of between about pH 6.0 and about pH 8.0, as described herein, after storage at 40° C. for at least 0.5 months, at least 1 months, at least 3 months, at least 6 months, or any combination thereof. In some embodiments, a liquid dosage form, as described herein, may comprise a pH of between about pH 6.0 and about pH 8.0, after storage at 60° C. for at least 0.5 months, at least 1 month, at least 1.5 months, or any combination thereof.

In some embodiments, a liquid dosage form, as described herein, after storage at 25° C. for at least 0.5 months, at least 1 months, at least 3 months, at least 6 months, at least 9 months, at least 12 months, or any combination thereof, may satisfy at least one of: (i) between about 90% and of about 110% of the naproxen sodium label claim contained therein; (ii) between about 90% and of about 110% of the methylparaben label claim contained therein; (iii) between about 90% and of about 110% of the propylparaben label claim contained therein; and (iv) any combination thereof.

In certain embodiments, a liquid dosage form, as described herein, after storage at 25° C. for at least 0.5 months, at least 1 months, at least 3 months, at least 6 months, at least 9 months, at least 12 months, or any combination thereof, may satisfy at least one of: (i) between about 90% and of about 105% of the naproxen sodium label claim contained therein; (ii) between about 90% and of about 105% of the methylparaben label claim contained therein; (iii) between about 90% and of about 105% of the propylparaben label claim contained therein; and (iv) any combination thereof.

In some embodiments, a liquid dosage form, as described herein, after storage at 25° C. for at least 0.5 months, at least 1 months, at least 3 months, at least 6 months, at least 9 months, at least 12 months, or any combination thereof, may satisfy at least one of: (i) between about 95% and of about 105% of the naproxen sodium label claim contained therein; (ii) between about 95% and of about 105% of the methylparaben label claim contained therein; (iii) between about 95% and of about 105% of the propylparaben label claim contained therein; and (iv) any combination thereof.

In some embodiments, a liquid dosage form, as described herein, after storage at 40° C. for at least 0.5 months, at least 1 months, at least 3 months, at least 6 months, or any combination thereof, may satisfy at least one of: (i) between about 90% and of about 110% of the naproxen sodium label claim contained therein; (ii) between about 90% and of about 110% of the methylparaben label claim contained therein; (iii) between about 90% and of about 110% of the propylparaben label claim contained therein; and (iv) any combination thereof.

In certain embodiments, a liquid dosage form, as described herein, after storage at 40° C. for at least 0.5 months, at least 1 months, at least 3 months, at least 6 months, or any combination thereof, may satisfy at least one of: (i) between about 90% and of about 105% of the naproxen sodium label claim contained therein; (ii) between about 90% and of about 105% of the methylparaben label claim contained therein; (iii) between about 90% and of about 105% of the propylparaben label claim contained therein; and (iv) any combination thereof.

In certain embodiments, a liquid dosage form, as described herein, after storage at 40° C. for at least 0.5 months, at least 1 months, at least 3 months, at least 6 months, or any combination thereof, may satisfy at least one of: (i) between about 95% and of about 105% of the naproxen sodium label claim contained therein; (ii) between about 90% and of about 100% of the methylparaben label claim contained therein; (iii) between about 95% and of about 105% of the propylparaben label claim contained therein; and (iv) any combination thereof.

In some embodiments, a liquid dosage form, as described herein, after storage at 60° C. for at least 0.5 months, at least 1 month, at least 1.5 months, or any combination thereof, may satisfy at least one of: (i) between about 90% and of about 110% of the naproxen sodium label claim contained therein; (ii) between about 80% and of about 110% of the methylparaben label claim contained therein; (iii) between about 90% and of about 110% of the propylparaben label claim contained therein; and (iv) any combination thereof.

In certain embodiments, a liquid dosage form, as described herein, after storage at 60° C. for at least 0.5 months, at least 1 month, at least 1.5 months, or any combination thereof, may satisfy at least one of: (i) between about 90% and of about 105% of the naproxen sodium label claim contained therein; (ii) between about 80% and of about 100% of the methylparaben label claim contained therein; (iii) between about 90% and of about 105% of the propylparaben label claim contained therein; and (iv) any combination thereof.

In some embodiments, a liquid dosage form, as described herein, after storage at 60° C. for at least 0.5 months, at least 1 month, at least 1.5 months, or any combination thereof, may satisfy at least one of: (i) between about 95% and of about 105% of the naproxen sodium label claim contained therein; (ii) between about 80% and of about 95% of the methylparaben label claim contained therein; (iii) between about 95% and of about 105% of the propylparaben label claim contained therein; and (iv) any combination thereof.

In certain embodiments, a liquid dosage form, as described herein, may be formulated to be as a sugar-free formulation. When a liquid dosage form, as described herein, is formulated to be a sugar-free formulation, the liquid dosage form will not contain any compound selected from the group consisting of arabinose, dextrin, dextrose, disaccharide, fructose, fucose, galactose, glucose, lactose, maltodextrin, maltose, mannose, rhamnose, ribose, saccharose, sucrose, trehalose, and xylose.

In some embodiments, a liquid dosage form, as described herein, may be formulated to be as a gluten-free formulation. When a liquid dosage form, as described herein, is formulated to be a gluten-free formulation, the liquid dosage form will not contain gluten.

In certain embodiments, a liquid dosage form, as described herein, may be formulated to be as a dye-free formulation. When a liquid dosage form, as described herein, is formulated to be a dye-free formulation, the liquid dosage form will not contain any dyes.

In some embodiments, a liquid dosage form, as described herein, may be formulated to be as an alcohol-free formulation. When a liquid dosage form, as described herein, is formulated to be an alcohol-free formulation, the liquid dosage form will contain, at most, 0.50% ethanol.

In certain embodiments, a liquid dosage form, as described herein, may be formulated to be substantially free of parabens. When a liquid dosage form, as described herein, is formulated to be substantially free of parabens, the liquid dosage form will contain, at most, 0.10% of propylparaben, methylparaben, butylparaben, ethylparaben, isobutylparaben, isopropylparaben, benzylparaben, and pentylparaben.

In some embodiments, a liquid dosage form, as described herein, may be formulated to be non-gritty. When a liquid dosage form, as described herein, is formulated to be non-gritty, the liquid dosage form will not have a gritty mouthful when a subject consumes the liquid dosage form.

In certain embodiments, a liquid dosage form, as described herein, may be formulated to have a desirable taste profile. When a liquid dosage form, as described herein, is formulated to have a desirable taste profile, the liquid dosage form will not be bitter, salty, sour, metallic tasting, lye-like tasting, or any combination of the foregoing.

In some embodiments, a liquid dosage form, as described herein, may be formulated to have a desirable chemesthetic profile. When a liquid dosage form, as described herein, is formulated to have a desirable chemesthetic profile, the liquid dosage form will not cause any irritation, burning, stinging, pricking, itching, tingling, numbness, tickling, or any combination thereof, of the oral cavity and/or pharyngeal cavity of a subject when the liquid dosage form is consumed by the subject.

In some embodiments, a liquid dosage form, as described herein, may be administered to a subject to treat, ameliorate, prevent, and/or reduce the severity of acute pain or a symptom thereof.

In certain embodiments, a liquid dosage form, as described herein, may be administered to a subject to treat, ameliorate, prevent, and/or reduce the severity of chronic pain or a symptom thereof.

In some embodiments, a liquid dosage form, as described herein, may be administered to a subject to treat, ameliorate, prevent, and/or reduce the severity of acute inflammation or a symptom thereof.

In certain embodiments, a liquid dosage form, as described herein, may be administered to a subject to treat, ameliorate, prevent, and/or reduce the severity of chronic inflammation or a symptom thereof.

In some embodiments, a liquid dosage form, as described herein, may be administered to a subject to treat, ameliorate, prevent, and/or reduce the severity of a fever or a symptom thereof.

In certain embodiments, a liquid dosage form, as described herein, may be administered to a subject to treat, ameliorate, prevent, and/or reduce the severity of blood clots or a symptom thereof.

In some embodiments, a liquid dosage form, as described herein, may be administered to a subject to treat, ameliorate, prevent, and/or reduce the severity of a heart attack or a symptom thereof.

In certain embodiments, a liquid dosage form, as described herein, may be administered to a subject to treat, ameliorate, prevent, and/or reduce the severity of a stroke or a symptom thereof.

In some embodiments, a liquid dosage form, as described herein, may be administered to a subject to treat, ameliorate, prevent, and/or reduce the severity of osteoarthritis or a symptom thereof.

In certain embodiments, a liquid dosage form, as described herein, may be administered to a subject to treat, ameliorate, prevent, and/or reduce the severity of rheumatoid arthritis or a symptom thereof.

In some embodiments, a liquid dosage form, as described herein, may be administered to a subject to treat, ameliorate, prevent, and/or reduce the severity of headaches or a symptom thereof.

In certain embodiments, a liquid dosage form, as described herein, may be administered to a subject to treat, ameliorate, prevent, and/or reduce the severity of migraines or a symptom thereof.

In some embodiments, a liquid dosage form, as described herein, may be administered to a subject to treat, ameliorate, prevent, and/or reduce the severity of gout or a symptom thereof.

In certain embodiments, a liquid dosage form, as described herein, may be administered to a subject to treat, ameliorate, prevent, and/or reduce the severity of dysmenorrhea or a symptom thereof.

In some embodiments, a liquid dosage form, as described herein, may be administered to a subject to treat, ameliorate, prevent, and/or reduce the severity of muscle stiffness or a symptom thereof.

In certain embodiments, a liquid dosage form, as described herein, may be administered to a subject to treat, ameliorate, prevent, and/or reduce the severity of ileus or a symptom thereof.

In some embodiments, a liquid dosage form, as described herein, may be administered to a subject to treat, ameliorate, prevent, and/or reduce the severity of macular edema or a symptom thereof.

In certain embodiments, a liquid dosage form, as described herein, may be administered to a subject to temporarily relieve minor aches and pains due to arthritis.

In some embodiments, a liquid dosage form, as described herein, may be administered to a subject to temporarily relieve minor aches and pains due to muscular aches.

In certain embodiments, a liquid dosage form, as described herein, may be administered to a subject to temporarily relieve minor aches and pains due to backache.

In some embodiments, a liquid dosage form, as described herein, may be administered to a subject to temporarily relieve minor aches and pains due to menstrual cramps.

In certain embodiments, a liquid dosage form, as described herein, may be administered to a subject to temporarily relieve minor aches and pains due to headache.

In some embodiments, a liquid dosage form, as described herein, may be administered to a subject to temporarily relieve minor aches and pains due to toothache.

In certain embodiments, a liquid dosage form, as described herein, may be administered to a subject to temporarily relieve minor aches and pains due to the common cold.

In some embodiments, a liquid dosage form, as described herein, may be administered to a subject to temporarily reduce fever.

In certain embodiments, a subject, as described herein, may be a child. In some embodiments, a subject, as described herein, may be an adult. In certain embodiments, a subject, as described herein, may be a senior citizen, wherein a senior citizen is an adult that is at least 65 years old.

In some embodiments, a liquid dosage form, as described herein, may be formulated to administer a dose of an NSAID, as described herein, within the range of 1 mg, 5 mg, 10 mg 15 mg, 20 mg, 25 mg, 30 mg, 35 mg, 40 mg, 45 mg, 50 mg, 55 mg, 60 mg, 65 mg, 70 mg, 75 m, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, 1010 mg, 1020 mg, 1030 mg, 1040 mg, 1050 mg, 1060 mg, 1070 mg, 1080 mg, 1090 mg, 1100 mg, 1110 mg, 1120 mg, 1130 mg, 1140 mg, 1150 mg, 1160 mg, 1170 mg, 1180 mg, 1190 mg, 1200 mg, 1210 mg, 1220 mg, 1230 mg, 1240 mg, 1250 mg, 1260 mg, 1270 mg, 1280 mg, 1290 mg, 1300 mg, 1310 mg, 1320 mg, 1330 mg, 1340 mg, 1350 mg, 1360 mg, 1370 mg, 1380 mg, 1390 mg, 1400 mg, 1410 mg, 1420 mg, 1430 mg, 1440 mg, 1450 mg, 1460 mg, 1470 mg, 1480 mg, 1490 mg, 1500 mg, 1510 mg, 1520 mg, 1530 mg, 1540 mg, 1550 mg, 1560 mg, 1570 mg, 1580 mg, 1590 mg, 1600 mg, 1610 mg, 1620 mg, 1630 mg, 1640 mg, 1650 mg, 1660 mg, 1670 mg, 1680 mg, 1690 mg, 1700 mg, 1710 mg, 1720 mg, 1730 mg, 1740 mg, 1750 mg, 1760 mg, 1770 mg, 1780 mg, 1790 mg, 1800 mg, 1810 mg, 1820 mg, 1830 mg, 1840 mg, 1850 mg, 1860 mg, 1870 mg, 1880 mg, 1890 mg, 1900 mg, 1910 mg, 1920 mg, 1930 mg, 1940 mg, 1950 mg, 1960 mg, 1970 mg, 1980 mg, 1990 mg, 2000 mg, or any fraction or integer in between any two of the preceding amounts.

In certain embodiments, a liquid dosage form, as described herein, may be administered to a subject, as described herein, on an hourly basis, e.g., every—one, two, three, four, five, six, seven, eight, nine, ten, eleven, twelve, thirteen, fourteen, fifteen, sixteen, seventeen, eighteen, nineteen, twenty, twenty-one, twenty-two, twenty-three hours, or any interval in between. In some embodiments, a liquid dosage form, as described herein, may be administered to a subject, as described herein, once a day, every two days, every three days, every four days, every five days, every six days, every week, every eight days, every nine days, every ten days, every two weeks, every month, or more or less frequently, as needed to achieve the desired therapeutic effect. The frequency of administration can be tailored to specific subjects in view of the disclosure contained herein. One of skill in the art would immediately envisage the appropriate dosage amount and regimen in view of the disclosure contained herein.

Some embodiments provide a liquid dosage form comprising an amount of an in situ IEX-NSAID complex (“in situ liquid dosage form”). As used herein, the term “in situ IEX-NSAID complex” refers to a compound comprising at least one NSAID or salt thereof, as described herein, bound to an IEX rein, as described herein, that is formed via a reaction that occurs within the in situ liquid dosage form after providing an amount of an NSAID or salt thereof, as described herein, and an amount of an IEX resin, as described herein, therein.

In certain embodiments, an in situ liquid dosage form, as described herein, may be adapted to be orally administered to a subject. In situ liquid dosage forms falling within the scope of disclosure contemplated herein include, but are not limited to aqueous dispersions, oil-based dispersions, aqueous suspensions, oil-based suspensions, aqueous solutions, oil-based solutions, beverages, syrups, elixirs, emulsions, and the like. In certain embodiments, an in situ liquid dosage form, as described herein, may be an aqueous dispersion, an aqueous suspension, or an aqueous solution. In some embodiments, an in situ liquid dosage form, as described herein, may be an aqueous suspension.

In some embodiments, an in situ liquid dosage form, as described herein, may comprise an amount of NSAID or salt thereof in an in situ IEX-NSAID complex, as described herein, that is about 1.0% wt./vol. to about 5.0% wt./vol. (relative to the volume of the in situ liquid dosage form). In certain embodiments, an in situ liquid dosage form, as described herein, may comprise an amount of NSAID or salt thereof in an in situ IEX-NSAID complex, as described herein that is about 1.5% wt./vol. to about 4.0% wt./vol. (relative to the volume of the in situ liquid dosage form). In certain embodiments, an in situ liquid dosage form, as described herein, may comprise an amount of NSAID or salt thereof in an in situ IEX-NSAID complex, as described herein, that is about 2.0% wt./vol. to about 3.0% wt./vol. (relative to the volume of the in situ liquid dosage form). In some embodiments, an in situ liquid dosage form, as described herein, may comprise an amount of NSAID or salt thereof in an in situ IEX-NSAID complex, as described herein, that is about 2.0% wt./vol. to about 2.5% wt./vol. (relative to the volume of the in situ liquid dosage form).

In certain embodiments, an in situ IEX-NSAID complex, as described herein, may comprise between about 38% NSAID or salt thereof by weight and about 60% NSAID or salt thereof by weight. In some embodiments, an in situ IEX-NSAID complex, as described herein, may comprise between about 42% NSAID or salt thereof by weight and about 56% NSAID or salt thereof by weight. In certain embodiments, an in situ IEX-NSAID complex, as described herein, may comprise between about 46% NSAID or salt thereof by weight and about 52% NSAID or salt thereof by weight.

In some embodiments, an in situ IEX-NSAID complex, as described herein, may comprise a bound-NSAID efficiency. As used herein, the term “bound-NSAID efficiency” refers to the amount of NSAID or salt thereof, as described herein, that is bound to the IEX resin, as described herein, compared to the amount of NSAID or salt thereof that was initially provided in the in situ liquid dosage form, as described herein, expressed as a percentage. In certain embodiments, a bound-NSAID efficiency, as described herein, may be at least about 80%. In certain embodiments, a bound-NSAID efficiency, as described herein, may be at least about 85%. In some embodiments, a bound-NSAID efficiency, as described herein, may be at least about 90%. In certain embodiments, a bound-NSAID efficiency, as described herein, may be at least about 95%.

In some embodiments, an in situ IEX-NSAID complex, as described herein, may release at least about 50% of the bound NSAID or salt thereof within 10 minutes during a dissolution test. In some embodiments, an in situ IEX-NSAID complex, as described herein, may release at least about 50% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an in situ IEX-NSAID complex, as described herein, may release at least about 60% of the bound NSAID or salt thereof within 10 minutes during a dissolution test. In some embodiments, an in situ IEX-NSAID complex, as described herein, may release at least about 60% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an in situ IEX-NSAID complex, as described herein, may release at least about 70% of the bound NSAID or salt thereof within 10 minutes during a dissolution test. In some embodiments, an in situ IEX-NSAID complex, as described herein, may release at least about 70% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an in situ IEX-NSAID complex, as described herein, may release at least about 80% of the bound NSAID or salt thereof within 10 minutes during a dissolution test. In some embodiments, an in situ IEX-NSAID complex, as described herein, may release at least about 80% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an in situ IEX-NSAID complex, as described herein, may release at least about 75% of the bound NSAID or salt thereof within 30 minutes during a dissolution test. In some embodiments, an in situ IEX-NSAID complex, as described herein, may release at least about 75% of the bound NSAID or salt thereof within 30 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an in situ IEX-NSAID complex, as described herein, may release at least about 80% of the bound NSAID or salt thereof, as described herein, within 30 minutes during a dissolution test. In some embodiments, an in situ IEX-NSAID complex, as described herein, may release at least about 80% of the bound NSAID or salt thereof within 30 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an in situ IEX-NSAID complex, as described herein, may release at least about 85% of the bound NSAID or salt thereof within 30 minutes during a dissolution test. In some embodiments, an in situ IEX-NSAID complex, as described herein, may release at least about 85% of the bound NSAID or salt thereof within 30 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an in situ IEX-NSAID complex, as described herein, may release at least about 80% of the bound NSAID or salt thereof within 60 minutes during a dissolution test. In some embodiments, an in situ IEX-NSAID complex, as described herein, may release at least about 80% of the bound NSAID or salt thereof within 60 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an in situ IEX-NSAID complex, as described herein, may release at least about 85% of the bound NSAID or salt thereof within 60 minutes during a dissolution test. In some embodiments, an in situ IEX-NSAID complex, as described herein, may release at least about 85% of the bound NSAID or salt thereof within 60 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, an in situ IEX-NSAID complex, as described herein, may release at least about 90% of the bound NSAID or salt thereof within 60 minutes during a dissolution test. In some embodiments, an in situ IEX-NSAID complex, as described herein, may release at least about 90% of the bound NSAID or salt thereof within 60 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, an in situ liquid dosage form, as described herein, may comprise an unbound-NSAID concentration. As used herein, the term “unbound-NSAID concentration” refers to the concentration of NSAID or salt thereof, as described herein, that is present in the in situ liquid dosage form and not bound to IEX resin. In some embodiments, an unbound-NSAID concentration, as described herein, may be less than about 5 mg/mL. In certain embodiments, an unbound-NSAID concentration, as described herein, may be less than about 4 mg/mL. In some embodiments, an unbound-NSAID concentration, as described herein, may be less than about 3 mg/mL. In certain embodiments, an unbound-NSAID concentration, as described herein, may be less than about 2 mg/mL.

In certain embodiments, an in situ liquid dosage form, as described herein, may comprise an amount of one more or more sweeteners. As used herein, the term “sweetener,” refers to any compound that increases the sweetness of an in situ liquid dosage form, as described herein, when included therein. Sweeteners falling within the scope of disclosure contemplated herein include, but are not limited to acesulfame potassium, advantame, arabinose, aspartame, cyclamate, dextrin, dextrose, disaccharide, erythritol, fructose, fucose, galactose, glucose, isomalt, lactitol, lactose, maltitol, maltodextrin, maltose, mannitol, mannose, monkfruit extract, neotame, rhamnose, ribose, saccharin, saccharose, sorbitol, steviol glycosides, sucrose, sucralose, thaumatin, trehalose, xylitol, and xylose, as well as any other known sweetener not expressly listed that would be readily envisaged by one skilled the art.

In some embodiments, an in situ liquid dosage form, as described herein, may comprise an amount of xylitol, an amount of sucralose, or the combination thereof.

In certain embodiments, an in situ liquid dosage form, as described herein, may comprise an amount of xylitol that is about 5% wt./vol. to about 40% wt./vol. (relative to the volume of the in situ liquid dosage form), such as, for example, 5% wt./vol., 7.5% wt./vol., 10% wt./vol., 12.5% wt./vol., 15% wt./vol., 17.5% wt./vol., 20% wt./vol., 22.5% wt./vol., 25% wt./vol., 27.5% wt./vol., 30% wt./vol., 32.5% wt./vol., 35% wt./vol., 37.5% wt./vol., 40% wt./vol., or any value in between any two of the preceding amounts.

In some embodiments, an in situ liquid dosage form, as described herein, may comprise an amount of sucralose that is about 0.025% wt./vol. to about 1.0% wt./vol. (relative to the volume of the in situ liquid dosage form), such as, for example, 0.025% wt./vol., 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., 0.625% wt./vol., 0.65% wt./vol., 0.675% wt./vol., 0.70% wt./vol., 0.725% wt./vol., 0.75% wt./vol., 0.775% wt./vol., 0.80% wt./vol., 0.825% wt./vol., 0.85% wt./vol., 0.875% wt./vol., 0.90% wt./vol., 0.925% wt./vol., 0.95% wt./vol., 0.975% wt./vol., 1.0% wt./vol., or any value in between any two of the preceding amounts.

In some embodiments, an in situ liquid dosage form, as described herein, may comprise an amount of one more or more thickeners. As used herein, the term “thickener,” refers to any compound that increases the viscosity of an in situ liquid dosage form, as described herein, when included therein. Thickeners falling within the scope of disclosure contemplated herein include, but are not limited to acacia, agar, alginates, alginic acid, alumina, aluminum monostearate, amidated pectins, bentonite, carbomers, carboxymethylcellulose, carrageenan, caseinates, cellulose, chitosan, dextran, dextrin, ethylcellulose, ethylene vinyl acetates, fatty acids, gelatin, gellan gum, glycerin, guar gum, hectorite, hydroxyethylcellulose, hydroxymethylcellulose, hydroxypropylcellulose, hydroxypropyl methylcellulose, karaya gum, locust bean, magnesia, magnesium aluminometasilicates, such as Neusilin®, magnesium aluminum silicates, methylcellulose, microcrystalline cellulose, pectin, polyethylene glycol, polyethylene oxide, propylene glycol, propylene glycol alginate, polyvinyl alcohol, polyvinylpyrrolidones, pullulan, silica, silicones, starches, tragacanth, xanthan gum, any salt of the foregoing, and any hydrate of the foregoing, as well as other known thickeners not expressly listed that would be readily envisaged by one skilled the art.

In certain embodiments, an in situ liquid dosage form, as described herein, may comprise an amount of glycerin, an amount of xanthan gum, an amount of a magnesium aluminometasilicate or a hydrate thereof, or any combination thereof.

In some embodiments, an in situ liquid dosage form, as described herein, may comprise an amount of glycerin that is about 5% wt./vol. to about 50% wt./vol. (relative to the volume of the in situ liquid dosage form), such as, for example, 5.0% wt./vol., 7.5% wt./vol., 10.0% wt./vol., 12.5% wt./vol., 15.0% wt./vol., 17.5% wt./vol., 20.0% wt./vol., 22.5% wt./vol., 25.0% wt./vol., 27.5% wt./vol., 30.0% wt./vol., 32.5% wt./vol., 35.0% wt./vol., 37.5% wt./vol., 40.0% wt./vol., 42.5% wt./vol., 45.0% wt./vol., 47.5% wt./vol., 50.0% wt./vol., or any value in between any two of the preceding amounts.

In some embodiments, an in situ liquid dosage form, as described herein, may comprise an amount of xanthan gum that is about 0.05% wt./vol. to about 1.0% wt./vol. (relative to the volume of the in situ liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., 0.625% wt./vol., 0.65% wt./vol., 0.675% wt./vol., 0.70% wt./vol., 0.725% wt./vol., 0.75% wt./vol., 0.775% wt./vol., 0.80% wt./vol., 0.825% wt./vol., 0.85% wt./vol., 0.875% wt./vol., 0.90% wt./vol., 0.925% wt./vol., 0.95% wt./vol., 0.975% wt./vol., 1.0% wt./vol., or any value in between any two of the preceding amounts.

In some embodiments, an in situ liquid dosage form, as described herein, may comprise an amount of a magnesium aluminometasilicate or hydrate thereof, that is about 0.05% wt./vol. to about 0.50% wt./vol. (relative to the volume of the in situ liquid dosage form), such as for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In some embodiments, an in situ liquid dosage form, as described herein, may comprise one or more of: (i) an amount of glycerin that is about 5% wt./vol. to about 50% wt./vol. (relative to the volume of the liquid dosage form), such as, for example, 5.0% wt./vol., 7.5% wt./vol., 10.0% wt./vol., 12.5% wt./vol., 15.0% wt./vol., 17.5% wt./vol., 20.0% wt./vol., 22.5% wt./vol., 25.0% wt./vol., 27.5% wt./vol., 30.0% wt./vol., 32.5% wt./vol., 35.0% wt./vol., 37.5% wt./vol., 40.0% wt./vol., 42.5% wt./vol., 45.0% wt./vol., 47.5% wt./vol., 50.0% wt./vol., or any value in between any two of the preceding amounts; (ii) an amount of xanthan gum that is about 0.05% wt./vol. to about 1.0% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., 0.625% wt./vol., 0.65% wt./vol., 0.675% wt./vol., 0.70% wt./vol., 0.725% wt./vol., 0.75% wt./vol., 0.775% wt./vol., 0.80% wt./vol., 0.825% wt./vol., 0.85% wt./vol., 0.875% wt./vol., 0.90% wt./vol., 0.925% wt./vol., 0.95% wt./vol., 0.975% wt./vol., 1.0% wt./vol., or any value in between any two of the preceding amounts; and (iii) an amount of a magnesium aluminometasilicate or hydrate thereof, that is about 0.05% wt./vol. to about 0.50% wt./vol. (relative to the volume of the liquid dosage form), such as for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In some embodiments, an in situ liquid dosage form, as described herein, may comprise an amount of one more or more preservatives. As used herein, the term “preservative,” refers to any compound that increases the improves the shelf life of an in situ liquid dosage form, as described herein, when included therein. Preservatives falling within the scope of disclosure contemplated herein include, but are not limited to alkali-/alkaline-metal benzoates (such as sodium benzoate, potassium benzoate, magnesium benzoate, calcium benzoate, and the like), alkali-/alkaline-metal metabisulfites (such as sodium metabisulfite, potassium metabisulfite, magnesium metabisulfite, calcium metabisulfite, and the like), alkali-/alkaline-metal sorbates (such as sodium sorbate, potassium sorbate, magnesium sorbate, calcium sorbate, and the like), benzoic acid, edetate sodium, hydroxyparabenzoate, parabens (such as methylparaben, ethylparaben, propylparaben, isopropylparaben, butylparaben, isobutylparaben, pentylparaben, benzylparaben, and the like), polysorbate 20, polysorbate 40, polysorbate 60, polysorbate 80, propylene glycol, propylene glycol diacetate, propylene glycol dicaprylate, propylene glycol monostearate, as well as other known preservatives not expressly listed that would be readily envisaged by one skilled the art.

In certain embodiments, an in situ liquid dosage form, as described herein, may comprise an amount of edetate sodium, methylparaben, polysorbate 80, potassium sorbate, propylene glycol, propylparaben, sodium benzoate, or any combination of the foregoing.

In some embodiments, an in situ liquid dosage form, as described herein, may comprise an amount of edetate sodium that is about 0.05% wt./vol. to about 0.25% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., or any value in between any two of the preceding amounts.

In certain embodiments, an in situ liquid dosage form, as described herein, may comprise an amount of methylparaben that is about 0.05% wt./vol. to about 0.50% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In some embodiments, an in situ liquid dosage form, as described herein, may comprise an amount of polysorbate 80 that is about 0.10% wt./vol. to about 0.60% wt./vol. (relative to the volume of the liquid dosage form), such as, for example, 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., or any value in between any two of the preceding amounts.

In certain embodiments, an in situ liquid dosage form, as described herein, may comprise an amount of potassium sorbate that is about 0.05% wt./vol. to about 0.50% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In some embodiments, an in situ liquid dosage form, as described herein, may comprise an amount of propylene glycol that is about 0.50% wt./vol. to about 10.0% wt./vol. (relative to the volume of the liquid dosage form), such as, for example, 0.50% wt./vol., 0.75% wt./vol., 1.0% wt./vol., 1.25% wt./vol., 1.5% wt./vol., 1.75% wt./vol., 2.0% wt./vol., 2.25% wt./vol., 2.5% wt./vol., 2.75% wt./vol., 3.0% wt./vol., 3.25% wt./vol., 3.5% wt./vol., 3.75% wt./vol., 4.0% wt./vol., 4.25% wt./vol., 4.5% wt./vol., 4.75% wt./vol., 5.0% wt./vol., 5.25% wt./vol., 5.5% wt./vol., 5.75% wt./vol., 6.0% wt./vol., 6.25% wt./vol., 6.5% wt./vol., 6.75% wt./vol., 7.0% wt./vol., 7.25% wt./vol., 7.5% wt./vol., 7.75% wt./vol., 8.0% wt./vol., 8.25% wt./vol., 8.5% wt./vol., 8.75% wt./vol., 9.0% wt./vol., 9.25% wt./vol., 9.5% wt./vol., 9.75% wt./vol., 10.0% wt./vol., or any value in between any two of the preceding amounts.

In certain embodiments, an in situ liquid dosage form, as described herein, may comprise an amount of propylparaben that is about 0.01% wt./vol. to about 0.30% wt./vol. (relative to the volume of the liquid dosage form), such as, for example, 0.01% wt./vol., 0.025% wt./vol., 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., or any value in between any two of the preceding amounts.

In some embodiments, an in situ liquid dosage form, as described herein, may comprise an amount of sodium benzoate that is about 0.10% wt./vol. to about 0.50% wt./vol. (relative to the volume of the liquid dosage form), such as, for example, 0.10% wt./vol., 0.12% wt./vol., 0.14% wt./vol., 0.16% wt./vol., 0.18% wt./vol., 0.20% wt./vol., 0.22% wt./vol., 0.24% wt./vol., 0.26% wt./vol., 0.28% wt./vol., 0.30% wt./vol., 0.32% wt./vol., 0.34% wt./vol., 0.36% wt./vol., 0.38% wt./vol., 0.40% wt./vol., 0.42% wt./vol., 0.44% wt./vol., 0.46% wt./vol., 0.48% wt./vol., 0.50% wt./vol., or any value in between any two of the preceding amounts.

In certain embodiments, an in situ liquid dosage form, as described herein, may comprise one or more of: (i) an amount of edetate sodium that is about 0.05% wt./vol. to about 0.25% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., or any value in between any two of the preceding amounts; (ii) an amount of methylparaben that is about 0.05% wt./vol. to about 0.50% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts; (iii) an amount of polysorbate 80 that is about 0.10% wt./vol. to about 0.60% wt./vol. (relative to the volume of the liquid dosage form), such as, for example, 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., or any value in between any two of the preceding amounts; (iv) an amount of potassium sorbate that is about 0.05% wt./vol. to about 0.50% wt./vol. (relative to the volume of the liquid dosage form), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts; (v) an amount of propylene glycol that is about 0.50% wt./vol. to about 10.0% wt./vol. (relative to the volume of the liquid dosage form), such as, for example, 0.50% wt./vol., 0.75% wt./vol., 1.0% wt./vol., 1.25% wt./vol., 1.5% wt./vol., 1.75% wt./vol., 2.0% wt./vol., 2.25% wt./vol., 2.5% wt./vol., 2.75% wt./vol., 3.0% wt./vol., 3.25% wt./vol., 3.5% wt./vol., 3.75% wt./vol., 4.0% wt./vol., 4.25% wt./vol., 4.5% wt./vol., 4.75% wt./vol., 5.0% wt./vol., 5.25% wt./vol., 5.5% wt./vol., 5.75% wt./vol., 6.0% wt./vol., 6.25% wt./vol., 6.5% wt./vol., 6.75% wt./vol., 7.0% wt./vol., 7.25% wt./vol., 7.5% wt./vol., 7.75% wt./vol., 8.0% wt./vol., 8.25% wt./vol., 8.5% wt./vol., 8.75% wt./vol., 9.0% wt./vol., 9.25% wt./vol., 9.5% wt./vol., 9.75% wt./vol., 10.0% wt./vol., or any value in between any two of the preceding amounts; (vi) an amount of propylparaben that is about 0.01% wt./vol. to about 0.30% wt./vol. (relative to the volume of the liquid dosage form), such as, for example, 0.01% wt./vol., 0.025% wt./vol., 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., or any value in between any two of the preceding amounts; and (vii) an amount of sodium benzoate that is about 0.10% wt./vol. to about 0.50% wt./vol. (relative to the volume of the liquid dosage form), such as, for example, 0.10% wt./vol., 0.12% wt./vol., 0.14% wt./vol., 0.16% wt./vol., 0.18% wt./vol., 0.20% wt./vol., 0.22% wt./vol., 0.24% wt./vol., 0.26% wt./vol., 0.28% wt./vol., 0.30% wt./vol., 0.32% wt./vol., 0.34% wt./vol., 0.36% wt./vol., 0.38% wt./vol., 0.40% wt./vol., 0.42% wt./vol., 0.44% wt./vol., 0.46% wt./vol., 0.48% wt./vol., 0.50% wt./vol., or any value in between any two of the preceding amounts.

In certain embodiments, an in situ liquid dosage form, as described herein, may comprise an amount of one more or more flavorants. As used herein, the term “flavorant,” refers to any compound that imparts a flavor to an in situ liquid dosage form, as described herein, when included therein. Flavorants falling within the scope of disclosure contemplated herein include, but are not limited to apple flavoring, apricot flavoring, banana flavoring, berry flavoring, blackberry flavoring, blueberry flavoring, bubble gum flavoring, cherry flavoring, chocolate flavoring, cinnamon flavoring, citrus flavoring, coconut flavoring, cotton candy flavoring, cranberry flavoring, fruit punch flavoring, grape flavoring, grapefruit flavoring, kiwi flavoring, lemon flavoring, lime flavoring, mango flavoring, mint flavoring, orange flavoring, peach flavoring, pineapple flavoring, plum flavoring, pomegranate flavoring, raspberry flavoring, strawberry flavoring, tangerine flavoring, vanilla flavoring, and watermelon flavoring.

In some embodiments, an in situ liquid dosage form, as described herein, may comprise an amount of flavorant that is about 0.10% wt./vol. to about 3.0% wt./vol. (relative to the volume of the in situ liquid dosage form), such as, for example, 0.10% wt./vol., 0.15% wt./vol., 0.20% wt./vol., 0.25% wt./vol., 0.30% wt./vol., 0.35% wt./vol., 0.40% wt./vol., 0.45% wt./vol., 0.50% wt./vol., 0.55% wt./vol., 0.60% wt./vol., 0.65% wt./vol., 0.70% wt./vol., 0.75% wt./vol., 0.80% wt./vol., 0.85% wt./vol., 0.90% wt./vol., 0.95% wt./vol., 1.0% wt./vol., 1.05% wt./vol., 1.10% wt./vol., 1.15% wt./vol., 1.20% wt./vol., 1.25% wt./vol., 1.30% wt./vol., 1.35% wt./vol., 1.40% wt./vol., 1.45% wt./vol., 1.50% wt./vol., 1.55% wt./vol., 1.60% wt./vol., 1.65% wt./vol., 1.70% wt./vol., 1.75% wt./vol., 1.80% wt./vol., 1.85% wt./vol., 1.90% wt./vol., 1.95% wt./vol., 2.0% wt./vol., 2.05% wt./vol., 2.10% wt./vol., 2.15% wt./vol., 2.20% wt./vol., 2.25% wt./vol., 2.30% wt./vol., 2.35% wt./vol., 2.40% wt./vol., 2.45% wt./vol., 2.50% wt./vol., 2.55% wt./vol., 2.60% wt./vol., 2.65% wt./vol., 2.70% wt./vol., 2.75% wt./vol., 2.80% wt./vol., 2.85% wt./vol., 2.90% wt./vol., 2.95% wt./vol., 3.0% wt./vol., or any value in between any two of the preceding amounts.

In some embodiments, an in situ liquid dosage form, as described herein, may comprise an amount of grape flavoring.

In certain embodiments, an in situ liquid dosage form, as described herein, may comprise an amount of grape flavoring that is about 0.10% wt./vol. to about 3.0% wt./vol. (relative to the volume of the in situ liquid dosage form), such as, for example, 0.10% wt./vol., 0.15% wt./vol., 0.20% wt./vol., 0.25% wt./vol., 0.30% wt./vol., 0.35% wt./vol., 0.40% wt./vol., 0.45% wt./vol., 0.50% wt./vol., 0.55% wt./vol., 0.60% wt./vol., 0.65% wt./vol., 0.70% wt./vol., 0.75% wt./vol., 0.80% wt./vol., 0.85% wt./vol., 0.90% wt./vol., 0.95% wt./vol., 1.0% wt./vol., 1.05% wt./vol., 1.10% wt./vol., 1.15% wt./vol., 1.20% wt./vol., 1.25% wt./vol., 1.30% wt./vol., 1.35% wt./vol., 1.40% wt./vol., 1.45% wt./vol., 1.50% wt./vol., 1.55% wt./vol., 1.60% wt./vol., 1.65% wt./vol., 1.70% wt./vol., 1.75% wt./vol., 1.80% wt./vol., 1.85% wt./vol., 1.90% wt./vol., 1.95% wt./vol., 2.0% wt./vol., 2.05% wt./vol., 2.10% wt./vol., 2.15% wt./vol., 2.20% wt./vol., 2.25% wt./vol., 2.30% wt./vol., 2.35% wt./vol., 2.40% wt./vol., 2.45% wt./vol., 2.50% wt./vol., 2.55% wt./vol., 2.60% wt./vol., 2.65% wt./vol., 2.70% wt./vol., 2.75% wt./vol., 2.80% wt./vol., 2.85% wt./vol., 2.90% wt./vol., 2.95% wt./vol., 3.0% wt./vol., or any value in between any two of the preceding amounts.

In some embodiments, an in situ liquid dosage form, as described herein, may comprise a density. In certain embodiments a density of an in situ liquid dosage form, as described herein, may be about 0.90 g/mL to about 1.30 g/mL, such as, for example 0.90 g/mL, 0.91 g/mL, 0.92 g/mL, 0.93 g/mL, 0.94 g/mL, 0.95 g/mL, 0.96 g/mL, 0.97 g/mL, 0.98 g/mL, 0.99 g/mL, 1.0 g/mL, 1.01 g/mL, 1.02 g/mL, 1.03 g/mL, 1.04 g/mL, 1.05 g/mL, 1.06 g/mL, 1.07 g/mL, 1.08 g/mL, 1.09 g/mL, 1.10 g/mL, 1.11 g/mL, 1.12 g/mL, 1.13 g/mL, 1.14 g/mL, 1.15 g/mL, 1.16 g/mL, 1.17 g/mL, 1.18 g/mL, 1.19 g/mL, 1.20 g/mL, 1.21 g/mL, 1.22 g/mL, 1.23 g/mL, 1.24 g/mL, 1.25 g/mL, 1.26 g/mL, 1.27 g/mL, 1.28 g/mL, 1.29 g/mL, 1.30 g/mL, or any value in between any two of the preceding amounts.

In certain embodiments, an in situ liquid dosage form, as described herein, may comprise a viscosity. In some embodiments a viscosity of an in situ liquid dosage form, as described herein, may be about 10 cP to about 1000 cP. In certain embodiments, a viscosity of an in situ liquid dosage form, as described herein, may be about 50 cP to about 800 cP. In some embodiments, a viscosity of an in situ liquid dosage form, as described herein, may be about 100 cP to about 700 cP. In certain embodiments, a viscosity of an in situ liquid dosage form, as described herein, may be about 300 cP to about 700 cP. In certain embodiments, a viscosity of an in situ liquid dosage form, as described herein, may be about 300 cP to about 700 cP. In some embodiments, a viscosity of an in situ liquid dosage form, as described herein, may be about 300 cP to about 700 cP after storage at 25° C. for at least 0.5 months, at least 1 months, at least 3 months, at least 6 months, at least 9 months, at least 12 months, or any combination thereof. In certain embodiments, a viscosity of an in situ liquid dosage form, as described herein, may be about 300 cP to about 700 cP after storage at 40° C. for at least 0.5 months, at least 1 months, at least 3 months, at least 6 months, or any combination thereof. In some embodiments, a viscosity of an in situ liquid dosage form, as described herein, may be about 300 cP to about 700 cP after storage at 60° C. for at least 0.5 months, at least 1 month, at least 1.5 months, or any combination thereof.

In some embodiments, an in situ liquid dosage form, as described herein, may comprise a dispersibility. In certain embodiments a dispersibility of an in situ liquid dosage form, as described herein, may be at least about 80%. In some embodiments, a dispersibility of an in situ liquid dosage form, as described herein, may be at least about 85%. In certain embodiments, a dispersibility of an in situ liquid dosage form, as described herein, may be at least about 90%. In some embodiments, a dispersibility of an in situ liquid dosage form, as described herein, may be at least about 95%.

In some embodiments, an in situ liquid dosage form, as described herein, may pass a microbial test selected from USP <51> (14 day interval), USP <51> (28 day interval), USP <60>, USP <61>, USP <62>, and any combination thereof, after storage at 25° C. for at least 0.5 months, at least 1 months, at least 3 months, at least 6 months, at least 9 months, at least 12 months, or any combination thereof. In certain embodiments, an in situ liquid dosage form, as described herein, may pass a microbial test selected from USP <51> (14 day interval), USP <51> (28 day interval), USP <60>, USP <61>, USP <62>, and any combination thereof, after storage at 40° C. for at least 0.5 months, at least 1 months, at least 3 months, at least 6 months, or any combination thereof. In certain embodiments, an in situ liquid dosage form, as described herein, may pass a microbial test selected from USP <51> (14 day interval), USP <51> (28 day interval), USP <60>, USP <61>, USP <62>, and any combination thereof, after storage at 60° C. for at least 0.5 months, at least 1 month, at least 1.5 months, or any combination thereof.

In some embodiments, an in situ liquid dosage form, as described herein, after storage at 25° C. for at least 0.5 months, at least 1 months, at least 3 months, at least 6 months, at least 9 months, at least 12 months, or any combination thereof, may satisfy at least one of: (i) no more than about 0.2% of NRC A contained therein; (ii) no more than about 0.2% of NRC K contained therein; (iii) no more than about 0.2% of NRC L contained therein; (iv) no more than about 0.2% of any IUI contained therein; (v) no more than about 1.5% of Total Impurities contained therein; and (vi) any combination thereof.

In some embodiments, an in situ liquid dosage form, as described herein, after storage at 25° C. for at least 0.5 months, at least 1 months, at least 3 months, at least 6 months, at least 9 months, at least 12 months, or any combination thereof, may satisfy at least one of: (i) no more than about 0.1% of NRC A contained therein; (ii) no more than about 0.1% of NRC K contained therein; (iii) no more than about 0.1% of NRC L contained therein; (iv) no more than about 0.1% of any IUI contained therein; (v) no more than about 0.5% of Total Impurities contained therein; and (vi) any combination thereof.

In some embodiments, an in situ liquid dosage form, as described herein, after storage at 25° C. for at least 0.5 months, at least 1 months, at least 3 months, at least 6 months, at least 9 months, at least 12 months, or any combination thereof, may satisfy at least one of: (i) no more than about 0.05% of NRC A contained therein; (ii) no more than about 0.05% of NRC K contained therein; (iii) no more than about 0.05% of NRC L contained therein; (iv) no more than about 0.05% of any IUI contained therein; (v) no more than about 0.1% of Total Impurities contained therein; and (vi) any combination thereof.

In some embodiments, an in situ liquid dosage form, as described herein, after storage at 40° C. for at least 0.5 months, at least 1 months, at least 3 months, at least 6 months, or any combination thereof, may satisfy at least one of: (i) no more than about 0.2% of NRC A contained therein; (ii) no more than about 0.2% of NRC K contained therein; (iii) no more than about 0.2% of NRC L contained therein; (iv) no more than about 0.2% of any IUI contained therein; (v) no more than about 1.5% of Total Impurities contained therein; and (vi) any combination thereof.

In some embodiments, an in situ liquid dosage form, as described herein, after storage at 40° C. for at least 0.5 months, at least 1 months, at least 3 months, at least 6 months, or any combination thereof, may satisfy at least one of: (i) no more than about 0.1% of NRC A contained therein; (ii) no more than about 0.1% of NRC K contained therein; (iii) no more than about 0.1% of NRC L contained therein; (iv) no more than about 0.1% of any IUI contained therein; (v) no more than about 0.5% of Total Impurities contained therein; and (vi) any combination thereof.

In some embodiments, an in situ liquid dosage form, as described herein, after storage at 40° C. for at least 0.5 months, at least 1 months, at least 3 months, at least 6 months, or any combination thereof, may satisfy at least one of: (i) no more than about 0.05% of NRC A contained therein; (ii) no more than about 0.05% of NRC K contained therein; (iii) no more than about 0.05% of NRC L contained therein; (iv) no more than about 0.05% of any IUI contained therein; (v) no more than about 0.1% of Total Impurities contained therein; and (vi) any combination thereof.

In some embodiments, an in situ liquid dosage form, as described herein, after storage at 60° C. for at least 0.5 months, at least 1 month, at least 1.5 months, or any combination thereof, may satisfy at least one of: (i) no more than about 0.2% of NRC A contained therein; (ii) no more than about 0.2% of NRC K contained therein; (iii) no more than about 0.2% of NRC L contained therein; (iv) no more than about 0.2% of any IUI contained therein; (v) no more than about 1.5% of Total Impurities contained therein; and (vi) any combination thereof.

In some embodiments, an in situ liquid dosage form, as described herein, after storage at 60° C. for at least 0.5 months, at least 1 month, at least 1.5 months, or any combination thereof, may satisfy at least one of: (i) no more than about 0.1% of NRC A contained therein; (ii) no more than about 0.1% of NRC K contained therein; (iii) no more than about 0.1% of NRC L contained therein; (iv) no more than about 0.15% of any IUI contained therein; (v) no more than about 0.5% of Total Impurities contained therein; and (vi) any combination thereof.

In some embodiments, an in situ liquid dosage form, as described herein, after storage at 60° C. for at least 0.5 months, at least 1 month, at least 1.5 months, or any combination thereof, may satisfy at least one of: (i) no more than about 0.05% of NRC A contained therein; (ii) no more than about 0.05% of NRC K contained therein; (iii) no more than about 0.05% of NRC L contained therein; (iv) no more than about 0.1% of any IUI contained therein; (v) no more than about 0.1% of Total Impurities contained therein; and (vi) any combination thereof.

In some embodiments, an in situ liquid dosage form, as described herein, may comprise a pH of between about pH 6.0 and about pH 8.0, such as for example pH 6.0, pH 6.1, pH 6.2, pH 6.3, pH 6.4, pH 6.5, pH 6.6, pH 6.7, pH 6.8, pH 6.9, pH 7.0, pH 7.1, pH 7.2, pH 7.3, pH 7.4, pH 7.5, pH 7.6, pH 7.7, pH 7.8, pH 7.9, or pH 8.0. In certain embodiments, an in situ liquid dosage form, as described herein, may comprise a pH of between about pH 6.0 and about pH 8.0, as described herein, after storage at 25° C. for at least 0.5 months, at least 1 months, at least 3 months, at least 6 months, at least 9 months, at least 12 months, or any combination thereof. In some embodiments, an in situ liquid dosage form, as described herein, may comprise a pH of between about pH 6.0 and about pH 8.0, as described herein, after storage at 40° C. for at least 0.5 months, at least 1 months, at least 3 months, at least 6 months, or any combination thereof. In some embodiments, an in situ liquid dosage form, as described herein, may comprise a pH of between about pH 6.0 and about pH 8.0, after storage at 60° C. for at least 0.5 months, at least 1 month, at least 1.5 months, or any combination thereof.

In some embodiments, an in situ liquid dosage form, as described herein, after storage at 25° C. for at least 0.5 months, at least 1 months, at least 3 months, at least 6 months, at least 9 months, at least 12 months, or any combination thereof, may satisfy at least one of: (i) between about 90% and of about 110% of the naproxen sodium label claim contained therein; (ii) between about 90% and of about 110% of the methylparaben label claim contained therein; (iii) between about 90% and of about 110% of the propylparaben label claim contained therein; and (iv) any combination thereof.

In certain embodiments, an in situ liquid dosage form, as described herein, after storage at 25° C. for at least 0.5 months, at least 1 months, at least 3 months, at least 6 months, at least 9 months, at least 12 months, or any combination thereof, may satisfy at least one of: (i) between about 90% and of about 105% of the naproxen sodium label claim contained therein; (ii) between about 90% and of about 105% of the methylparaben label claim contained therein; (iii) between about 90% and of about 105% of the propylparaben label claim contained therein; and (iv) any combination thereof.

In some embodiments, an in situ liquid dosage form, as described herein, after storage at 25° C. for at least 0.5 months, at least 1 months, at least 3 months, at least 6 months, at least 9 months, at least 12 months, or any combination thereof, may satisfy at least one of: (i) between about 95% and of about 105% of the naproxen sodium label claim contained therein; (ii) between about 95% and of about 105% of the methylparaben label claim contained therein; (iii) between about 95% and of about 105% of the propylparaben label claim contained therein; and (iv) any combination thereof.

In some embodiments, an in situ liquid dosage form, as described herein, after storage at 40° C. for at least 0.5 months, at least 1 months, at least 3 months, at least 6 months, or any combination thereof, may satisfy at least one of: (i) between about 90% and of about 110% of the naproxen sodium label claim contained therein; (ii) between about 90% and of about 110% of the methylparaben label claim contained therein; (iii) between about 90% and of about 110% of the propylparaben label claim contained therein; and (iv) any combination thereof.

In certain embodiments, an in situ liquid dosage form, as described herein, after storage at 40° C. for at least 0.5 months, at least 1 months, at least 3 months, at least 6 months, or any combination thereof, may satisfy at least one of: (i) between about 90% and of about 105% of the naproxen sodium label claim contained therein; (ii) between about 90% and of about 105% of the methylparaben label claim contained therein; (iii) between about 90% and of about 105% of the propylparaben label claim contained therein; and (iv) any combination thereof.

In certain embodiments, an in situ liquid dosage form, as described herein, after storage at 40° C. for at least 0.5 months, at least 1 months, at least 3 months, at least 6 months, or any combination thereof, may satisfy at least one of: (i) between about 95% and of about 105% of the naproxen sodium label claim contained therein; (ii) between about 90% and of about 100% of the methylparaben label claim contained therein; (iii) between about 95% and of about 105% of the propylparaben label claim contained therein; and (iv) any combination thereof.

In some embodiments, an in situ liquid dosage form, as described herein, after storage at 60° C. for at least 0.5 months, at least 1 month, at least 1.5 months, or any combination thereof, may satisfy at least one of: (i) between about 90% and of about 110% of the naproxen sodium label claim contained therein; (ii) between about 80% and of about 110% of the methylparaben label claim contained therein; (iii) between about 90% and of about 110% of the propylparaben label claim contained therein; and (iv) any combination thereof.

In certain embodiments, an in situ liquid dosage form, as described herein, after storage at 60° C. for at least 0.5 months, at least 1 month, at least 1.5 months, or any combination thereof, may satisfy at least one of: (i) between about 90% and of about 105% of the naproxen sodium label claim contained therein; (ii) between about 80% and of about 100% of the methylparaben label claim contained therein; (iii) between about 90% and of about 105% of the propylparaben label claim contained therein; and (iv) any combination thereof.

In some embodiments, an in situ liquid dosage form, as described herein, after storage at 60° C. for at least 0.5 months, at least 1 month, at least 1.5 months, or any combination thereof, may satisfy at least one of: (i) between about 95% and of about 105% of the naproxen sodium label claim contained therein; (ii) between about 80% and of about 95% of the methylparaben label claim contained therein; (iii) between about 95% and of about 105% of the propylparaben label claim contained therein; and (iv) any combination thereof.

In certain embodiments, an in situ liquid dosage form, as described herein, may be formulated to be as a sugar-free formulation. When an in situ liquid dosage form, as described herein, is formulated to be a sugar-free formulation, the in situ liquid dosage form will not contain any compound selected from the group consisting of arabinose, dextrin, dextrose, disaccharide, fructose, fucose, galactose, glucose, lactose, maltodextrin, maltose, mannose, rhamnose, ribose, saccharose, sucrose, trehalose, and xylose.

In some embodiments, an in situ liquid dosage form, as described herein, may be formulated to be as a gluten-free formulation. When an in situ liquid dosage form, as described herein, is formulated to be a gluten-free formulation, the in situ liquid dosage form will not contain gluten.

In certain embodiments, an in situ liquid dosage form, as described herein, may be formulated to be as a dye-free formulation. When an in situ liquid dosage form, as described herein, is formulated to be a dye-free formulation, the in situ liquid dosage form will not contain any dyes.

In some embodiments, an in situ liquid dosage form, as described herein, may be formulated to be as an alcohol-free formulation. When an in situ liquid dosage form, as described herein, is formulated to be an alcohol-free formulation, the in situ liquid dosage form will contain, at most, 0.50% ethanol.

In certain embodiments, an in situ liquid dosage form, as described herein, may be formulated to be substantially free of parabens. When an in situ liquid dosage form, as described herein, is formulated to be substantially free of parabens, the in situ liquid dosage form will contain, at most, 0.10% of propylparaben, methylparaben, butylparaben, ethylparaben, isobutylparaben, isopropylparaben, benzylparaben, and pentylparaben.

In some embodiments, an in situ liquid dosage form, as described herein, may be formulated to be non-gritty. When an in situ liquid dosage form, as described herein, is formulated to be non-gritty, the in situ liquid dosage form will not have a gritty mouthfeel when the in situ liquid dosage form is consumed by a subject.

In certain embodiments, an in situ liquid dosage form, as described herein, may be formulated to have a desirable taste profile. When an in situ liquid dosage form, as described herein, is formulated to have a desirable taste profile, the in situ liquid dosage form will not be bitter, salty, sour, metallic tasting, lye-like tasting, or any combination of the foregoing.

In some embodiments, an in situ liquid dosage form, as described herein, may be formulated to have a desirable chemesthetic profile. When an in situ liquid dosage form, as described herein, is formulated to have a desirable chemesthetic profile, the in situ liquid dosage form will not cause any irritation, burning, stinging, pricking, itching, tingling, numbness, tickling, or any combination thereof, of the oral cavity and/or pharyngeal cavity of a subject when the in situ liquid dosage form is consumed by the subject.

In some embodiments, an in situ liquid dosage form, as described herein, may be administered to a subject to treat, ameliorate, prevent, and/or reduce the severity of acute pain or a symptom thereof.

In certain embodiments, an in situ liquid dosage form, as described herein, may be administered to a subject to treat, ameliorate, prevent, and/or reduce the severity of chronic pain or a symptom thereof.

In some embodiments, an in situ liquid dosage form, as described herein, may be administered to a subject to treat, ameliorate, prevent, and/or reduce the severity of acute inflammation or a symptom thereof.

In certain embodiments, an in situ liquid dosage form, as described herein, may be administered to a subject to treat, ameliorate, prevent, and/or reduce the severity of chronic inflammation or a symptom thereof.

In some embodiments, an in situ liquid dosage form, as described herein, may be administered to a subject to treat, ameliorate, prevent, and/or reduce the severity of a fever or a symptom thereof.

In certain embodiments, an in situ liquid dosage form, as described herein, may be administered to a subject to treat, ameliorate, prevent, and/or reduce the severity of blood clots or a symptom thereof.

In some embodiments, an in situ liquid dosage form, as described herein, may be administered to a subject to treat, ameliorate, prevent, and/or reduce the severity of a heart attack or a symptom thereof.

In certain embodiments, an in situ liquid dosage form, as described herein, may be administered to a subject to treat, ameliorate, prevent, and/or reduce the severity of a stroke or a symptom thereof.

In some embodiments, an in situ liquid dosage form, as described herein, may be administered to a subject to treat, ameliorate, prevent, and/or reduce the severity of osteoarthritis or a symptom thereof.

In certain embodiments, an in situ liquid dosage form, as described herein, may be administered to a subject to treat, ameliorate, prevent, and/or reduce the severity of rheumatoid arthritis or a symptom thereof.

In some embodiments, an in situ liquid dosage form, as described herein, may be administered to a subject to treat, ameliorate, prevent, and/or reduce the severity of headaches or a symptom thereof.

In certain embodiments, an in situ liquid dosage form, as described herein, may be administered to a subject to treat, ameliorate, prevent, and/or reduce the severity of migraines or a symptom thereof.

In some embodiments, an in situ liquid dosage form, as described herein, may be administered to a subject to treat, ameliorate, prevent, and/or reduce the severity of gout or a symptom thereof.

In certain embodiments, an in situ liquid dosage form, as described herein, may be administered to a subject to treat, ameliorate, prevent, and/or reduce the severity of dysmenorrhea or a symptom thereof.

In some embodiments, an in situ liquid dosage form, as described herein, may be administered to a subject to treat, ameliorate, prevent, and/or reduce the severity of muscle stiffness or a symptom thereof.

In certain embodiments, an in situ liquid dosage form, as described herein, may be administered to a subject to treat, ameliorate, prevent, and/or reduce the severity of ileus or a symptom thereof.

In some embodiments, an in situ liquid dosage form, as described herein, may be administered to a subject to treat, ameliorate, prevent, and/or reduce the severity of macular edema or a symptom thereof.

In certain embodiments, an in situ liquid dosage form, as described herein, may be administered to a subject to temporarily relieve minor aches and pains due to arthritis.

In some embodiments, an in situ liquid dosage form, as described herein, may be administered to a subject to temporarily relieve minor aches and pains due to muscular aches.

In certain embodiments, an in situ liquid dosage form, as described herein, may be administered to a subject to temporarily relieve minor aches and pains due to backache.

In some embodiments, an in situ liquid dosage form, as described herein, may be administered to a subject to temporarily relieve minor aches and pains due to menstrual cramps.

In certain embodiments, an in situ liquid dosage form, as described herein, may be administered to a subject to temporarily relieve minor aches and pains due to headache.

In some embodiments, an in situ liquid dosage form, as described herein, may be administered to a subject to temporarily relieve minor aches and pains due to toothache.

In certain embodiments, an in situ liquid dosage form, as described herein, may be administered to a subject to temporarily relieve minor aches and pains due to the common cold.

In some embodiments, an in situ liquid dosage form, as described herein, may be administered to a subject to temporarily reduce fever.

In certain embodiments, a subject, as described herein, may be a child. In some embodiments, a subject, as described herein, may be an adult. In certain embodiments, a subject, as described herein, may be a senior citizen, wherein a senior citizen is an adult that is at least 65 years old.

In some embodiments, an in situ liquid dosage form, as described herein, may be formulated to administer a dose of an NSAID, as described herein, within the range of 1 mg, 5 mg, 10 mg 15 mg, 20 mg, 25 mg, 30 mg, 35 mg, 40 mg, 45 mg, 50 mg, 55 mg, 60 mg, 65 mg, 70 mg, 75 m, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, 1010 mg, 1020 mg, 1030 mg, 1040 mg, 1050 mg, 1060 mg, 1070 mg, 1080 mg, 1090 mg, 1100 mg, 1110 mg, 1120 mg, 1130 mg, 1140 mg, 1150 mg, 1160 mg, 1170 mg, 1180 mg, 1190 mg, 1200 mg, 1210 mg, 1220 mg, 1230 mg, 1240 mg, 1250 mg, 1260 mg, 1270 mg, 1280 mg, 1290 mg, 1300 mg, 1310 mg, 1320 mg, 1330 mg, 1340 mg, 1350 mg, 1360 mg, 1370 mg, 1380 mg, 1390 mg, 1400 mg, 1410 mg, 1420 mg, 1430 mg, 1440 mg, 1450 mg, 1460 mg, 1470 mg, 1480 mg, 1490 mg, 1500 mg, 1510 mg, 1520 mg, 1530 mg, 1540 mg, 1550 mg, 1560 mg, 1570 mg, 1580 mg, 1590 mg, 1600 mg, 1610 mg, 1620 mg, 1630 mg, 1640 mg, 1650 mg, 1660 mg, 1670 mg, 1680 mg, 1690 mg, 1700 mg, 1710 mg, 1720 mg, 1730 mg, 1740 mg, 1750 mg, 1760 mg, 1770 mg, 1780 mg, 1790 mg, 1800 mg, 1810 mg, 1820 mg, 1830 mg, 1840 mg, 1850 mg, 1860 mg, 1870 mg, 1880 mg, 1890 mg, 1900 mg, 1910 mg, 1920 mg, 1930 mg, 1940 mg, 1950 mg, 1960 mg, 1970 mg, 1980 mg, 1990 mg, 2000 mg, or any fraction or integer in between any two of the preceding amounts.

In certain embodiments, an in situ liquid dosage form, as described herein, may be administered to a subject, as described herein, on an hourly basis, e.g., every—one, two, three, four, five, six, seven, eight, nine, ten, eleven, twelve, thirteen, fourteen, fifteen, sixteen, seventeen, eighteen, nineteen, twenty, twenty-one, twenty-two, twenty-three hours, or any interval in between. In some embodiments, an in situ liquid dosage form, as described herein, may be administered to a subject, as described herein, once a day, every two days, every three days, every four days, every five days, every six days, every week, every eight days, every nine days, every ten days, every two weeks, every month, or more or less frequently, as needed to achieve the desired therapeutic effect. The frequency of administration can be tailored to specific subjects in view of the disclosure contained herein. One of skill in the art would immediately envisage the appropriate dosage amount and regimen in view of the disclosure contained herein.

Some embodiments provide a method of making an IEX-NSAID complex, as described herein.

In some embodiments, a method of making an IEX-NSAID complex, as described herein, may comprise the steps of: (i) providing an amount of NSAID or salt thereof, as described herein, in an aqueous solution; (ii) providing an amount of an IEX resin, as described herein, in the aqueous solution; (iii) optionally, adjusting the pH of the aqueous solution to a binding pH; (iv) mixing the aqueous solution at a mixing temperature for an amount of mixing time; (v) collecting a precipitant after the amount mixing time has elapsed; (vi) washing the precipitant with a volume aqueous solution; and (vii) dying the precipitant at a drying temperature for an amount of drying time.

In certain embodiments, an amount of NSAID or salt thereof provided in an aqueous solution, as described herein, may be between about 1.1 mg NSAID or salt thereof and about 1250 mg NSAID or salt thereof per mL of aqueous solution. In some embodiments, an amount of NSAID or salt thereof provided in an aqueous solution, as described herein, may be between about 1.1 mg NSAID or salt thereof and about 110 mg NSAID or salt thereof per mL of aqueous solution. In certain embodiments, an amount of NSAID or salt thereof provided in an aqueous solution, as described herein, may be between about 22 mg NSAID or salt thereof and about 88 mg NSAID or salt thereof per mL of aqueous solution.

In some embodiments, an amount of IEX resin provided in the aqueous solution, as described herein, may be between about 1.1 mg IEX resin and about 1250 mg IEX resin per mL of aqueous solution. In certain embodiments, an amount of NSAID or salt thereof provided in an aqueous solution, as described herein, may be between about 1.1 mg IEX resin and about 220 mg IEX resin per mL of aqueous solution. In some embodiments, an amount of IEX resin provided in the aqueous solution, as described herein, may be between about 22 mg IEX resin and about 176 mg IEX resin per mL of aqueous solution.

In certain embodiments, the amount of NSAID or salt thereof provided in an aqueous solution, as described herein, and the amount of IEX resin provided in the aqueous solution, as described herein, may define an NSAID-IEX resin ratio. In certain embodiments, an NSAID-IEX resin ratio, as described herein, may be between about 1:0.5 by mass and about 1:4 by mass (NSAID:IEX resin). In some embodiments, an NSAID-IEX resin ratio, as described herein, may be between about 1:0.5 by mass and about 1:2 by mass (NSAID:IEX resin). In certain embodiments, an NSAID-IEX resin ratio, as described herein, may be between about 1:1 by mass and about 1:2 by mass (NSAID:IEX resin). In some embodiments, an NSAID-IEX resin ratio, as described herein, may be between about 1:1.25 by mass and about 1:1.5 by mass (NSAID:IEX resin).

In some embodiments, a binding pH, as described herein, may be between about pH 7.0 and about pH 10. In certain embodiments, a binding pH, as described herein, may be between about pH 7.5 and about pH 9.5. In some embodiments, a binding pH, as described herein, may be between about pH 8.1 and about pH 9.2.

In some embodiments, a mixing temperature, as described herein, may be about 20° C. to about 60° C. In certain embodiments, a mixing temperature, as described herein, may be about 20° C. to about 40° C. In some embodiments, a mixing temperature, as described herein, may be about 20° C. to about 30° C. In certain embodiments, a mixing temperature, as described herein, may be about 20° C. to about 25° C.

In some embodiments, a mixing time, as described herein may be about 0.5 hours to about 6 hours. In certain embodiments, a mixing time, as described herein, may be about 0.5 hours to about 4 hours. In some embodiments, a mixing time, as described herein may be about 0.5 hours to about 2 hours. In certain embodiments, a mixing time, as described herein, may be about 0.5 hours to about 1 hour.

In some embodiments, a washing step, as described herein, may comprise washing the precipitant, as described herein, at least one time with a volume of aqueous solution. In certain embodiments, a washing step, as described herein, may comprise washing the precipitant, as described herein, at least two times with a volume of aqueous solution. The volume of aqueous solution used to perform a washing step, as described herein, is not particularly limited, though preferably, the volume of aqueous solution used to perform a washing step may be at least 50% of the volume of the aqueous solution in which the NSAID or salt thereof, as described herein, and the IEX resin, as described herein, were provided.

In some embodiments, a drying temperature, as described herein, may be about 50° C. to about 100° C. In certain embodiments, a drying temperature, as described herein, may be about 60° C. to about 90° C. In some embodiments, a drying temperature, as described herein, may be about 70° C. to about 80° C.

In certain embodiments, a drying time, as described herein, may be about 1 hour to about 10 hours. In some embodiments, a drying time, as described herein, may be about 3 hours to about 8 hours. In certain embodiments, a drying time, as described herein, may be about 5 hour to about 7 hours.

In some embodiments, a method of making an IEX-NSAID complex, as described herein, may comprise an NSAID-binding efficiency. As used herein, the term “NSAID-binding efficiency,” refers to the amount of NSAID or salt thereof, as described herein, that is bound to the IEX resin, as described herein, compared to the amount of NSAID or salt thereof that was provided in the aqueous solution, as described herein, after the amount mixing time, as described herein, has elapsed, expressed as a percentage. In some embodiments, an NSAID-binding efficiency, as described herein, may be at least about 80%. In certain embodiments, an NSAID-binding efficiency, as described herein, may be at least about 85%. In some embodiments, an NSAID-binding efficiency, as described herein, may be at least about 90%. In certain embodiments, an NSAID-binding efficiency, as described herein, may be at least about 95%.

In some embodiments, a method of making an IEX-NSAID complex, as described herein, may comprise a supernatant NSAID concentration. As used herein, the term “supernatant NSAID concentration,” refers to the concentration of NSAID or salt thereof, as described herein, that remains in the aqueous solution after the amount mixing time, as described herein, has elapsed. In some embodiments, a supernatant NSAID concentration, as described herein, may be less than about 5 mg of NSAID or salt thereof per mL of aqueous solution. In certain embodiments, a supernatant NSAID concentration, as described herein, may be less than about 4 mg of NSAID or salt thereof per mL of aqueous solution. In some embodiments, a supernatant NSAID concentration, as described herein, may be less than about 3 mg of NSAID or salt thereof per mL of aqueous solution. In certain embodiments, a supernatant NSAID concentration, as described herein, may be less than about 2 mg of NSAID or salt thereof per mL of aqueous solution.

In some embodiments, a method of making an IEX-NSAID complex, as described herein, may produce an IEX-NSAID complex that comprises between about 38% NSAID or salt thereof by weight and about 60% NSAID or salt thereof by weight. In certain embodiments, a method of making an IEX-NSAID complex, as described herein, may produce an IEX-NSAID complex that comprises between about 42% NSAID or salt thereof by weight and about 56% NSAID or salt thereof by weight. In some embodiments, a method of making an IEX-NSAID complex, as described herein, may produce an IEX-NSAID complex that comprises between about 46% NSAID or salt thereof by weight and about 52% NSAID or salt thereof by weight.

In some embodiments, a method of making an IEX-NSAID complex, as described herein, may produce an IEX-NSAID complex that releases at least about 50% of the bound NSAID or salt thereof within 10 minutes during a dissolution test. In certain embodiments, a method of making an IEX-NSAID complex, as described herein, may produce an IEX-NSAID complex that releases at least about 60% of the bound NSAID or salt thereof within 10 minutes during a dissolution test. In some embodiments, a method of making an IEX-NSAID complex, as described herein, may produce an IEX-NSAID complex that releases at least about 70% of the bound NSAID or salt thereof within 10 minutes during a dissolution test. In certain embodiments, a method of making an IEX-NSAID complex, as described herein, may produce an IEX-NSAID complex that releases at least about 80% of the bound NSAID or salt thereof within 10 minutes during a dissolution test.

In some embodiments, a method of making an IEX-NSAID complex, as described herein, may produce an IEX-NSAID complex that releases at least about 75% of the bound NSAID or salt thereof within 30 minutes during a dissolution test. In certain embodiments, a method of making an IEX-NSAID complex, as described herein, may produce an IEX-NSAID complex that releases at least about 80% of the bound NSAID or salt thereof within 30 minutes during a dissolution test. In some embodiments, a method of making an IEX-NSAID complex, as described herein, may produce an IEX-NSAID complex that releases at least about 85% of the bound NSAID or salt thereof within 30 minutes during a dissolution test.

In certain embodiments, a method of making an IEX-NSAID complex, as described herein, may produce an IEX-NSAID complex that releases at least about 80% of the bound NSAID or salt thereof within 60 minutes during a dissolution test. In some embodiments, a method of making an IEX-NSAID complex, as described herein, may produce an IEX-NSAID complex that releases at least about 85% of the bound NSAID or salt thereof within 60 minutes during a dissolution test. In some embodiments, a method of making an IEX-NSAID complex, as described herein, may produce an IEX-NSAID complex that releases at least about 90% of the bound NSAID or salt thereof within 60 minutes during a dissolution test.

Some embodiments provide a method of making an in situ liquid dosage form, as described herein, comprising an amount of an in situ IEX-NSAID complex, as described herein.

In some embodiments, a method of making an in situ liquid dosage form, as described herein, may comprise the steps of: (i) providing an amount of NSAID or salt thereof, as described herein, in an aqueous solution; (ii) providing an amount of an IEX resin, as described herein, in the aqueous solution; (iii) providing an amount of one or more sweeteners, as described herein, in the aqueous solution; (iv) providing an amount of one or more thickeners, as described herein, in the aqueous solution; (v) providing an amount of one or more preservatives, as described herein, in the aqueous solution; (vi) providing an amount of a flavorant, as described herein, in the aqueous solution; (vii) optionally, adjusting the pH of the aqueous solution to a binding pH; and (vii) mixing the aqueous solution at a mixing temperature for an amount of total mixing time.

In certain embodiments, an amount of NSAID or salt thereof provided in an aqueous solution, as described herein, may be about 1.0% wt./vol. to about 5.0% wt./vol. (relative to the volume of the aqueous solution). In some embodiments, an amount of NSAID or salt thereof provided in an aqueous solution, as described herein, may be about 1.5% wt./vol. to about 4.0% wt./vol. (relative to the volume of the aqueous solution). In certain embodiments, an amount of NSAID or salt thereof provided in an aqueous solution, as described herein, may be about 2.0% wt./vol. to about 3.0% wt./vol. (relative to the volume of the aqueous solution). In some embodiments, an amount of NSAID or salt thereof provided in an aqueous solution, as described herein, may be about 2.0% wt./vol. to about 2.5% wt./vol. (relative to the volume of the aqueous solution).

In certain embodiments, an amount of IEX resin provided in an aqueous solution, as described herein, may be about 1.0% wt./vol. to about 6.5% wt./vol. (relative to the volume of the aqueous solution). In some embodiments, an amount of IEX resin provided in an aqueous solution, as described herein, may be about 1.5% wt./vol. to about 6.0% wt./vol. (relative to the volume of the aqueous solution). In certain embodiments, an amount of IEX resin provided in an aqueous solution, as described herein, may be about 2.0% wt./vol. to about 4.0% wt./vol. (relative to the volume of the aqueous solution). In some embodiments, an amount of IEX resin provided in an aqueous solution, as described herein, may be about 2.5% wt./vol. to about 3.5% wt./vol. (relative to the volume of the aqueous solution).

In some embodiments, the amount of NSAID or salt thereof provided in an aqueous solution, as described herein, and the amount of IEX resin provided in the aqueous solution, as described herein, may define an NSAID-IEX resin ratio. In certain embodiments, an NSAID-IEX resin ratio, as described herein, may be between about 1:0.5 by mass and about 1:4 by mass (NSAID:IEX resin). In some embodiments, an NSAID-IEX resin ratio, as described herein, may be between about 1:0.5 by mass and about 1:2 by mass (NSAID:IEX resin). In certain embodiments, an NSAID-IEX resin ratio, as described herein, may be between about 1:1 by mass and about 1:2 by mass (NSAID:IEX resin). In some embodiments, an NSAID-IEX resin ratio, as described herein, may be between about 1:1.25 by mass and about 1:1.5 by mass (NSAID:IEX resin).

In certain embodiments, providing an amount of one or more sweeteners in an aqueous solution, as described herein, may comprise providing an amount of xylitol in an aqueous solution.

In some embodiments, an amount of xylitol provided in an aqueous solution, as described herein, may be about 5% wt./vol. to about 40% wt./vol. (relative to the volume of the aqueous solution), such as, for example, 5% wt./vol., 7.5% wt./vol., 10% wt./vol., 12.5% wt./vol., 15% wt./vol., 17.5% wt./vol., 20% wt./vol., 22.5% wt./vol., 25% wt./vol., 27.5% wt./vol., 30% wt./vol., 32.5% wt./vol., 35% wt./vol., 37.5% wt./vol., 40% wt./vol., or any value in between any two of the preceding amounts.

In some embodiments, providing an amount of one or more sweeteners in an aqueous solution, as described herein, may comprise providing an amount of sucralose in an aqueous solution.

In certain embodiments, an amount of sucralose provided in an aqueous solution, as described herein, may be about 0.025% wt./vol. to about 1.0% wt./vol. (relative to the volume of the aqueous solution), such as, for example, 0.025% wt./vol., 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., 0.625% wt./vol., 0.65% wt./vol., 0.675% wt./vol., 0.70% wt./vol., 0.725% wt./vol., 0.75% wt./vol., 0.775% wt./vol., 0.80% wt./vol., 0.825% wt./vol., 0.85% wt./vol., 0.875% wt./vol., 0.90% wt./vol., 0.925% wt./vol., 0.95% wt./vol., 0.975% wt./vol., 1.0% wt./vol., or any value in between any two of the preceding amounts.

In some embodiments, providing an amount of one or more thickeners in an aqueous solution, as described herein, may comprise providing an amount of glycerin in an aqueous solution. In certain embodiments, an amount of glycerin provided in an aqueous solution, as described herein, may be about 5% wt./vol. to about 50% wt./vol. (relative to the volume of the aqueous solution), such as, for example, 5.0% wt./vol., 7.5% wt./vol., 10.0% wt./vol., 12.5% wt./vol., 15.0% wt./vol., 17.5% wt./vol., 20.0% wt./vol., 22.5% wt./vol., 25.0% wt./vol., 27.5% wt./vol., 30.0% wt./vol., 32.5% wt./vol., 35.0% wt./vol., 37.5% wt./vol., 40.0% wt./vol., 42.5% wt./vol., 45.0% wt./vol., 47.5% wt./vol., 50.0% wt./vol., or any value in between any two of the preceding amounts.

In certain embodiments, providing an amount of one or more thickeners in an aqueous solution, as described herein, may comprise providing an amount of xanthan gum in an aqueous solution. In some embodiments, an amount of xanthan gum provided in an aqueous solution, as described herein, may be about 0.05% wt./vol. to about 1.0% wt./vol. (relative to the volume of the aqueous solution), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., 0.625% wt./vol., 0.65% wt./vol., 0.675% wt./vol., 0.70% wt./vol., 0.725% wt./vol., 0.75% wt./vol., 0.775% wt./vol., 0.80% wt./vol., 0.825% wt./vol., 0.85% wt./vol., 0.875% wt./vol., 0.90% wt./vol., 0.925% wt./vol., 0.95% wt./vol., 0.975% wt./vol., 1.0% wt./vol., or any value in between any two of the preceding amounts.

In some embodiments, providing an amount of one or more thickeners in an aqueous solution, as described herein, may comprise providing an amount of magnesium aluminometasilicate or a hydrate thereof in an aqueous solution. In certain embodiments, an amount of magnesium aluminometasilicate or a hydrate thereof provided in an aqueous solution, as described herein, may be about 0.05% wt./vol. to about 0.50% wt./vol. (relative to the volume of the aqueous solution), such as for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In certain embodiments, providing an amount of one or more thickeners in an aqueous solution, as described herein, may comprise providing an amount of an amount of glycerin, an amount of xanthan gum, and an amount of magnesium aluminometasilicate or a hydrate thereof in an aqueous solution. In some embodiments, an amount of glycerin provided in an aqueous solution, as described herein, may be about 5% wt./vol. to about 50% wt./vol. (relative to the volume of the aqueous solution), such as, for example, 5.0% wt./vol., 7.5% wt./vol., 10.0% wt./vol., 12.5% wt./vol., 15.0% wt./vol., 17.5% wt./vol., 20.0% wt./vol., 22.5% wt./vol., 25.0% wt./vol., 27.5% wt./vol., 30.0% wt./vol., 32.5% wt./vol., 35.0% wt./vol., 37.5% wt./vol., 40.0% wt./vol., 42.5% wt./vol., 45.0% wt./vol., 47.5% wt./vol., 50.0% wt./vol., or any value in between any two of the preceding amounts; an amount of xanthan gum provided in an aqueous solution, as described herein, may be about 0.05% wt./vol. to about 1.0% wt./vol. (relative to the volume of the aqueous solution), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., 0.625% wt./vol., 0.65% wt./vol., 0.675% wt./vol., 0.70% wt./vol., 0.725% wt./vol., 0.75% wt./vol., 0.775% wt./vol., 0.80% wt./vol., 0.825% wt./vol., 0.85% wt./vol., 0.875% wt./vol., 0.90% wt./vol., 0.925% wt./vol., 0.95% wt./vol., 0.975% wt./vol., 1.0% wt./vol., or any value in between any two of the preceding amounts; and an amount of magnesium aluminometasilicate or a hydrate thereof provided in an aqueous solution, as described herein, may be about 0.05% wt./vol. to about 0.50% wt./vol. (relative to the volume of the aqueous solution), such as for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In certain embodiments, providing an amount of one or more preservatives in an aqueous solution, as described herein, may comprise providing an amount of edetate sodium in an aqueous solution. In certain embodiments, an amount of edetate sodium provided in an aqueous solution, as described herein, may be about 0.05% wt./vol. to about 0.25% wt./vol. (relative to the volume of the aqueous solution), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., or any value in between any two of the preceding amounts.

In some embodiments, providing an amount of one or more preservatives in an aqueous solution, as described herein, may comprise providing an amount of methylparaben in an aqueous solution. In certain embodiments, an amount of methylparaben provided in an aqueous solution, as described herein, may be about 0.05% wt./vol. to about 0.50% wt./vol. (relative to the volume of the aqueous solution), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In certain embodiments, providing an amount of one or more preservatives in an aqueous solution, as described herein, may comprise providing an amount of polysorbate 80 in an aqueous solution. In certain embodiments, an amount of polysorbate 80 provided in an aqueous solution, as described herein, may be about 0.10% wt./vol. to about 0.60% wt./vol. (relative to the volume of the aqueous solution), such as, for example, 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., or any value in between any two of the preceding amounts.

In some embodiments, providing an amount of one or more preservatives in an aqueous solution, as described herein, may comprise providing an amount of potassium sorbate in an aqueous solution. In certain embodiments, an amount of potassium sorbate provided in an aqueous solution, as described herein, may be about 0.05% wt./vol. to about 0.50% wt./vol. (relative to the volume of the aqueous solution), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts.

In certain embodiments, providing an amount of one or more preservatives in an aqueous solution, as described herein, may comprise providing an amount of propylene glycol in an aqueous solution. In some embodiments, an amount of propylene glycol provided in an aqueous solution, as described herein, may be about 0.50% wt./vol. to about 10.0% wt./vol. (relative to the volume of the aqueous solution), such as, for example, 0.50% wt./vol., 0.75% wt./vol., 1.0% wt./vol., 1.25% wt./vol., 1.5% wt./vol., 1.75% wt./vol., 2.0% wt./vol., 2.25% wt./vol., 2.5% wt./vol., 2.75% wt./vol., 3.0% wt./vol., 3.25% wt./vol., 3.5% wt./vol., 3.75% wt./vol., 4.0% wt./vol., 4.25% wt./vol., 4.5% wt./vol., 4.75% wt./vol., 5.0% wt./vol., 5.25% wt./vol., 5.5% wt./vol., 5.75% wt./vol., 6.0% wt./vol., 6.25% wt./vol., 6.5% wt./vol., 6.75% wt./vol., 7.0% wt./vol., 7.25% wt./vol., 7.5% wt./vol., 7.75% wt./vol., 8.0% wt./vol., 8.25% wt./vol., 8.5% wt./vol., 8.75% wt./vol., 9.0% wt./vol., 9.25% wt./vol., 9.5% wt./vol., 9.75% wt./vol., 10.0% wt./vol., or any value in between any two of the preceding amounts.

In some embodiments, providing an amount of one or more preservatives in an aqueous solution, as described herein, may comprise providing an amount of propylparaben in an aqueous solution. In some embodiments, an amount of propylparaben provided in an aqueous solution, as described herein, may be about 0.01% wt./vol. to about 0.30% wt./vol. (relative to the volume of the aqueous solution), such as, for example, 0.01% wt./vol., 0.025% wt./vol., 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., or any value in between any two of the preceding amounts.

In certain embodiments, providing an amount of one or more preservatives in an aqueous solution, as described herein, may comprise providing an amount of sodium benzoate in an aqueous solution. In some embodiments, an amount of sodium benzoate provided in an aqueous solution, as described herein, may be about 0.10% wt./vol. to about 0.50% wt./vol. (relative to the volume of the aqueous solution), such as, for example, 0.10% wt./vol., 0.12% wt./vol., 0.14% wt./vol., 0.16% wt./vol., 0.18% wt./vol., 0.20% wt./vol., 0.22% wt./vol., 0.24% wt./vol., 0.26% wt./vol., 0.28% wt./vol., 0.30% wt./vol., 0.32% wt./vol., 0.34% wt./vol., 0.36% wt./vol., 0.38% wt./vol., 0.40% wt./vol., 0.42% wt./vol., 0.44% wt./vol., 0.46% wt./vol., 0.48% wt./vol., 0.50% wt./vol., or any value in between any two of the preceding amounts. In some embodiments, providing an amount of one or more preservatives in an aqueous solution, as described herein, may comprise providing one or more of: (i) an amount of edetate sodium that is about 0.05% wt./vol. to about 0.25% wt./vol. (relative to the volume of the aqueous solution), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., or any value in between any two of the preceding amounts; (ii) an amount of methylparaben that is about 0.05% wt./vol. to about 0.50% wt./vol. (relative to the volume of the aqueous solution), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts; (iii) an amount of polysorbate 80 that is about 0.10% wt./vol. to about 0.60% wt./vol. (relative to the volume of the aqueous solution), such as, for example, 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., 0.525% wt./vol., 0.55% wt./vol., 0.575% wt./vol., 0.60% wt./vol., or any value in between any two of the preceding amounts; (iv) an amount of potassium sorbate that is about 0.05% wt./vol. to about 0.50% wt./vol. (relative to the volume of the aqueous solution), such as, for example, 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., 0.325% wt./vol., 0.35% wt./vol., 0.375% wt./vol., 0.40% wt./vol., 0.425% wt./vol., 0.45% wt./vol., 0.475% wt./vol., 0.50% wt./vol., or any value in between any two of the preceding amounts; (v) an amount of propylene glycol that is about 0.50% wt./vol. to about 10.0% wt./vol. (relative to the volume of the aqueous solution), such as, for example, 0.50% wt./vol., 0.75% wt./vol., 1.0% wt./vol., 1.25% wt./vol., 1.5% wt./vol., 1.75% wt./vol., 2.0% wt./vol., 2.25% wt./vol., 2.5% wt./vol., 2.75% wt./vol., 3.0% wt./vol., 3.25% wt./vol., 3.5% wt./vol., 3.75% wt./vol., 4.0% wt./vol., 4.25% wt./vol., 4.5% wt./vol., 4.75% wt./vol., 5.0% wt./vol., 5.25% wt./vol., 5.5% wt./vol., 5.75% wt./vol., 6.0% wt./vol., 6.25% wt./vol., 6.5% wt./vol., 6.75% wt./vol., 7.0% wt./vol., 7.25% wt./vol., 7.5% wt./vol., 7.75% wt./vol., 8.0% wt./vol., 8.25% wt./vol., 8.5% wt./vol., 8.75% wt./vol., 9.0% wt./vol., 9.25% wt./vol., 9.5% wt./vol., 9.75% wt./vol., 10.0% wt./vol., or any value in between any two of the preceding amounts; (vi) an amount of propylparaben that is about 0.01% wt./vol. to about 0.30% wt./vol. (relative to the volume of the aqueous solution), such as, for example, 0.01% wt./vol., 0.025% wt./vol., 0.05% wt./vol., 0.075% wt./vol., 0.10% wt./vol., 0.125% wt./vol., 0.15% wt./vol., 0.175% wt./vol., 0.20% wt./vol., 0.225% wt./vol., 0.25% wt./vol., 0.275% wt./vol., 0.30% wt./vol., or any value in between any two of the preceding amounts; and (vii) an amount of sodium benzoate that is about 0.10% wt./vol. to about 0.50% wt./vol. (relative to the volume of the aqueous solution), such as, for example, 0.10% wt./vol., 0.12% wt./vol., 0.14% wt./vol., 0.16% wt./vol., 0.18% wt./vol., 0.20% wt./vol., 0.22% wt./vol., 0.24% wt./vol., 0.26% wt./vol., 0.28% wt./vol., 0.30% wt./vol., 0.32% wt./vol., 0.34% wt./vol., 0.36% wt./vol., 0.38% wt./vol., 0.40% wt./vol., 0.42% wt./vol., 0.44% wt./vol., 0.46% wt./vol., 0.48% wt./vol., 0.50% wt./vol., or any value in between any two of the preceding amounts.

In some embodiments, an amount of a flavorant provided in an aqueous solution, as described herein, may be about 0.10% wt./vol. to about 3.0% wt./vol. (relative to the volume of the aqueous solution), such as, for example, 0.10% wt./vol., 0.15% wt./vol., 0.20% wt./vol., 0.25% wt./vol., 0.30% wt./vol., 0.35% wt./vol., 0.40% wt./vol., 0.45% wt./vol., 0.50% wt./vol., 0.55% wt./vol., 0.60% wt./vol., 0.65% wt./vol., 0.70% wt./vol., 0.75% wt./vol., 0.80% wt./vol., 0.85% wt./vol., 0.90% wt./vol., 0.95% wt./vol., 1.0% wt./vol., 1.05% wt./vol., 1.10% wt./vol., 1.15% wt./vol., 1.20% wt./vol., 1.25% wt./vol., 1.30% wt./vol., 1.35% wt./vol., 1.40% wt./vol., 1.45% wt./vol., 1.50% wt./vol., 1.55% wt./vol., 1.60% wt./vol., 1.65% wt./vol., 1.70% wt./vol., 1.75% wt./vol., 1.80% wt./vol., 1.85% wt./vol., 1.90% wt./vol., 1.95% wt./vol., 2.0% wt./vol., 2.05% wt./vol., 2.10% wt./vol., 2.15% wt./vol., 2.20% wt./vol., 2.25% wt./vol., 2.30% wt./vol., 2.35% wt./vol., 2.40% wt./vol., 2.45% wt./vol., 2.50% wt./vol., 2.55% wt./vol., 2.60% wt./vol., 2.65% wt./vol., 2.70% wt./vol., 2.75% wt./vol., 2.80% wt./vol., 2.85% wt./vol., 2.90% wt./vol., 2.95% wt./vol., 3.0% wt./vol., or any value in between any two of the preceding amounts.

In some embodiments, providing an amount of a flavorant in an aqueous solution, as described herein, may comprise providing an amount of grape flavoring in an aqueous solution.

In certain embodiments, an amount of grape flavoring provided in an aqueous solution, as described herein, may be about 0.10% wt./vol. to about 3.0% wt./vol. (relative to the volume of the aqueous solution), such as, for example, 0.10% wt./vol., 0.15% wt./vol., 0.20% wt./vol., 0.25% wt./vol., 0.30% wt./vol., 0.35% wt./vol., 0.40% wt./vol., 0.45% wt./vol., 0.50% wt./vol., 0.55% wt./vol., 0.60% wt./vol., 0.65% wt./vol., 0.70% wt./vol., 0.75% wt./vol., 0.80% wt./vol., 0.85% wt./vol., 0.90% wt./vol., 0.95% wt./vol., 1.0% wt./vol., 1.05% wt./vol., 1.10% wt./vol., 1.15% wt./vol., 1.20% wt./vol., 1.25% wt./vol., 1.30% wt./vol., 1.35% wt./vol., 1.40% wt./vol., 1.45% wt./vol., 1.50% wt./vol., 1.55% wt./vol., 1.60% wt./vol., 1.65% wt./vol., 1.70% wt./vol., 1.75% wt./vol., 1.80% wt./vol., 1.85% wt./vol., 1.90% wt./vol., 1.95% wt./vol., 2.0% wt./vol., 2.05% wt./vol., 2.10% wt./vol., 2.15% wt./vol., 2.20% wt./vol., 2.25% wt./vol., 2.30% wt./vol., 2.35% wt./vol., 2.40% wt./vol., 2.45% wt./vol., 2.50% wt./vol., 2.55% wt./vol., 2.60% wt./vol., 2.65% wt./vol., 2.70% wt./vol., 2.75% wt./vol., 2.80% wt./vol., 2.85% wt./vol., 2.90% wt./vol., 2.95% wt./vol., 3.0% wt./vol., or any value in between any two of the preceding amounts.

In certain embodiments, a binding pH, as described herein, may be between about pH 7.0 and about pH 10. In certain embodiments, a binding pH, as described herein, may be between about pH 7.5 and about pH 9.5. In some embodiments, a binding pH, as described herein, may be between about pH 8.1 and about pH 9.2.

In certain embodiments, a mixing temperature, as described herein, may be about 20° C. to about 60° C. In some embodiments, a mixing temperature, as described herein, may be about 20° C. to about 40° C. In certain embodiments, a mixing temperature, as described herein, may be about 20° C. to about 30° C. In some embodiments, a mixing temperature, as described herein, may be about 20° C. to about 25° C.

In certain embodiments, a total mixing time, as described herein may be about 0.5 hours to about 10 hours. In some embodiments, a total mixing time, as described herein, may be about 2 hour to about 10 hours. In certain embodiments, a total mixing time, as described herein may be about 4 hour to about 10 hours. In some embodiments, a total mixing time, as described herein, may be about 4 hour to about 8 hours.

In certain embodiments, a method of making an in situ liquid dosage form, as described herein, may comprise an NSAID-binding efficiency. As used herein, the term “NSAID-binding efficiency,” refers to the amount of NSAID or salt thereof, as described herein, that is bound to the IEX resin, as described herein, compared to the amount of NSAID or salt thereof that was provided in the aqueous solution, as described herein, after the amount mixing time, as described herein, has elapsed, expressed as a percentage. In some embodiments, an NSAID-binding efficiency, as described herein, may be at least about 80%. In certain embodiments, an NSAID-binding efficiency, as described herein, may be at least about 85%. In some embodiments, an NSAID-binding efficiency, as described herein, may be at least about 90%. In certain embodiments, an NSAID-binding efficiency, as described herein, may be at least about 95%.

In some embodiments, a method of making an in situ liquid dosage form, as described herein, may comprise a supernatant NSAID concentration. As used herein, the term “supernatant NSAID concentration,” refers to the concentration of NSAID or salt thereof, as described herein, that remains in the aqueous solution after the amount mixing time, as described herein, has elapsed. In certain embodiments, a supernatant NSAID concentration, as described herein, may be less than about 5 mg of NSAID or salt thereof per mL of aqueous solution. In some embodiments, a supernatant NSAID concentration, as described herein, may be less than about 4 mg of NSAID or salt thereof per mL of aqueous solution. In certain embodiments, a supernatant NSAID concentration, as described herein, may be less than about 3 mg of NSAID or salt thereof per mL of aqueous solution. In some embodiments, a supernatant NSAID concentration, as described herein, may be less than about 2 mg of NSAID or salt thereof per mL of aqueous solution.

In certain embodiments, a method of making an in situ liquid dosage form, as described herein, may produce an in situ IEX-NSAID complex that comprises between about 38% NSAID or salt thereof by weight and about 60% NSAID or salt thereof by weight. In some embodiments, a method of making an in situ liquid dosage form, as described herein, may produce an in situ IEX-NSAID complex that comprises between about 42% NSAID or salt thereof by weight and about 56% NSAID or salt thereof by weight. In certain embodiments, a method of making an in situ liquid dosage form, as described herein, may produce an in situ IEX-NSAID complex that comprises between about 46% NSAID or salt thereof by weight and about 52% NSAID or salt thereof by weight.

In some embodiments, a method of making an in situ liquid dosage form, as described herein, may produce an in situ IEX-NSAID complex that releases at least about 50% of the bound NSAID or salt thereof within 10 minutes during a dissolution test. In some embodiments, a method of making an in situ liquid dosage form, as described herein, may produce an in situ IEX-NSAID complex that releases at least about 50% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, a method of making an in situ liquid dosage form, as described herein, may produce an in situ IEX-NSAID complex that releases at least about 60% of the bound NSAID or salt thereof within 10 minutes during a dissolution test. In some embodiments, a method of making an in situ liquid dosage form, as described herein, may produce an in situ IEX-NSAID complex that releases at least about 60% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, a method of making an in situ liquid dosage form, as described herein, may produce an in situ IEX-NSAID complex that releases at least about 70% of the bound NSAID or salt thereof within 10 minutes during a dissolution test. In some embodiments, a method of making an in situ liquid dosage form, as described herein, may produce an in situ IEX-NSAID complex that releases at least about 70% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, a method of making an in situ liquid dosage form, as described herein, may produce an in situ IEX-NSAID complex that releases at least about 80% of the bound NSAID or salt thereof within 10 minutes during a dissolution test. In some embodiments, a method of making an in situ liquid dosage form, as described herein, may produce an in situ IEX-NSAID complex that releases at least about 80% of the bound NSAID or salt thereof within 10 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, a method of making an in situ liquid dosage form, as described herein, may produce an in situ IEX-NSAID complex that releases at least about 75% of the bound NSAID or salt thereof within 30 minutes during a dissolution test. In some embodiments, a method of making an in situ liquid dosage form, as described herein, may produce an in situ IEX-NSAID complex that releases at least about 75% of the bound NSAID or salt thereof within 30 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, a method of making an in situ liquid dosage form, as described herein, may produce an in situ IEX-NSAID complex that releases at least about 80% of the bound NSAID or salt thereof within 30 minutes during a dissolution test. In some embodiments, a method of making an in situ liquid dosage form, as described herein, may produce an in situ IEX-NSAID complex that releases at least about 80% of the bound NSAID or salt thereof within 30 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, a method of making an in situ liquid dosage form, as described herein, may produce an in situ IEX-NSAID complex that releases at least about 85% of the bound NSAID or salt thereof within 30 minutes during a dissolution test. In some embodiments, a method of making an in situ liquid dosage form, as described herein, may produce an in situ IEX-NSAID complex that releases at least about 85% of the bound NSAID or salt thereof within 30 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, a method of making an in situ liquid dosage form, as described herein, may produce an in situ IEX-NSAID complex that releases at least about 80% of the bound NSAID or salt thereof within 60 minutes during a dissolution test. In some embodiments, a method of making an in situ liquid dosage form, as described herein, may produce an in situ IEX-NSAID complex that releases at least about 80% of the bound NSAID or salt thereof within 60 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, a method of making an in situ liquid dosage form, as described herein, may produce an in situ IEX-NSAID complex that releases at least about 85% of the bound NSAID or salt thereof within 60 minutes during a dissolution test. In some embodiments, a method of making an in situ liquid dosage form, as described herein, may produce an in situ IEX-NSAID complex that releases at least about 85% of the bound NSAID or salt thereof within 60 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In some embodiments, a method of making an in situ liquid dosage form, as described herein, may produce an in situ IEX-NSAID complex that releases at least about 90% of the bound NSAID or salt thereof within 60 minutes during a dissolution test. In some embodiments, a method of making an in situ liquid dosage form, as described herein, may produce an in situ IEX-NSAID complex that releases at least about 90% of the bound NSAID or salt thereof within 60 minutes during a dissolution test after: (i) storage at 25° C. for at least 0.5 months, at least 1 months, at least 3 months, at least 6 months, at least 9 months, at least 12 months, or any combination thereof; (ii) storage at 40° C. for at least 0.5 months, at least 1 months, at least 3 months, at least 6 months, or any combination thereof; and/or (iii) storage at 60° C. for at least 0.5 months, at least 1 month, at least 1.5 months, or any combination thereof.

In certain embodiments, a method of making an in situ liquid dosage form, as described herein, may produce an in situ liquid dosage form with a viscosity of about 10 cP to about 1000 cP. In some embodiments, a method of making an in situ liquid dosage form, as described herein, may produce an in situ liquid dosage form with a viscosity of about 50 cP to about 800 cP. In certain embodiments, a method of making an in situ liquid dosage form, as described herein, may produce an in situ liquid dosage form with a viscosity of about 100 cP to about 700 cP. In some embodiments, a method of making an in situ liquid dosage form, as described herein, may produce an in situ liquid dosage form with a viscosity of about 300 cP to about 700 cP. In certain embodiments, a method of making an in situ liquid dosage form, as described herein, may produce an in situ liquid dosage form with a viscosity of about 300 cP to about 700 cP after storage at 25° C. for at least 0.5 months, at least 1 months, at least 3 months, at least 6 months, at least 9 months, at least 12 months, or any combination thereof. In some embodiments, a method of making an in situ liquid dosage form, as described herein, may produce an in situ liquid dosage form with a viscosity of about 300 cP to about 700 cP after storage at 40° C. for at least 0.5 months, at least 1 months, at least 3 months, at least 6 months, or any combination thereof. In certain embodiments, a method of making an in situ liquid dosage form, as described herein, may produce an in situ liquid dosage form with a viscosity of about 300 cP to about 700 cP after storage at 60° C. for at least 0.5 months, at least 1 month, at least 1.5 months, or any combination thereof.

In certain embodiments, a method of making an in situ liquid dosage form, as described herein, may produce an in situ liquid dosage form that is non-gritty. When an in situ liquid dosage form, as described herein, is formulated to be non-gritty, the in situ liquid dosage form will not have a gritty mouthful when the in situ liquid dosage form is consumed by a subject.

In some embodiments, a method of making an in situ liquid dosage form, as described herein, may produce an in situ liquid dosage form that has a desirable taste profile. When an in situ liquid dosage form, as described herein, is formulated to have a desirable taste profile, the in situ liquid dosage form will not be bitter, salty, sour, metallic tasting, lye-like tasting, or any combination of the foregoing.

In certain embodiments, a method of making an in situ liquid dosage form, as described herein, may produce an in situ liquid dosage form that has a desirable chemesthetic profile. When an in situ liquid dosage form, as described herein, is formulated to have a desirable chemesthetic profile, the in situ liquid dosage form will not cause any irritation, burning, stinging, pricking, itching, tingling, numbness, tickling, or any combination thereof, of the oral cavity and/or pharyngeal cavity of a subject when the in situ liquid dosage form is consumed by the subject.

In certain embodiments, a method of making an in situ liquid dosage form, as described herein, may be able to be performed at a 2 L scale. In some embodiments, a method of making an in situ liquid dosage form, as described herein, may be able to be performed at a 20 L scale. In certain embodiments, a method of making an in situ liquid dosage form, as described herein, may be able to be performed at a 185 L scale.

As used herein, the term “pharmaceutically acceptable solvent,” refers to water or aqueous buffer solutions that are physiologically compatible, or aqueous solutions containing organic solvents that are physiologically compatible. A non-comprehensive list of pharmaceutically acceptable solvents is provided in U.S. Department of Health & Human Services, Food & Drug Administration, “Guidance for Industry: Q3C Impurities: Residual Solvents,” December 1997 or its current issue.

As used herein, the term “excipient” refers to any compound that is part of a formulation that is not an active ingredient, i.e., one that has no relevant biological activity, and which is added to the formulation to provide specific characteristics to the dosage form, including by way of example, providing protection to the active ingredient from chemical degradation, facilitating release of a tablet or caplet from equipment in which it is formed, and so forth.

The term “pharmaceutical formulation,” “formulation,” “composition,” and the like, refer to preparations that are in such a form as to permit the biological activity of the active ingredients to be effective, and therefore may be administered to a subject for therapeutic use along with pharmaceutical use. The meaning of these terms will be clear to the skilled artisan based upon the context in which they are used.

A “therapeutically effective amount,” as used herein, includes within its meaning, a non-toxic but sufficient amount of a compound active ingredient or composition comprising the same for use in the embodiments disclosed herein to provide the desired therapeutic effect. Similarly, “an amount effective to” or “an effective amount,” as used herein, includes within its meaning, a non-toxic but sufficient amount of a compound active ingredient or composition comprising the same to provide the desired effect. A “therapeutically effective amount” or an “effective amount” includes amounts of compounds that would not be achievable through a standard or natural diet, but requires supplementation and dosing, as described herein, to achieve specific, non-natural outcomes as set forth herein, along with expanded utilization of any compounds originating from or derived from natural sources. The exact amount of the active ingredient required may depend on factors such as the species being treated, the age and general condition of the subject, the severity of the condition being treated, the particular composition being administered, the weight of the subject, the mode of administration, and so forth. Thus, it may not always be possible to specify an exact “effective amount.” However, for any given case, an appropriate “effective amount” would be immediately envisaged by the skilled artisan in view of the context of the usage of the term “effective amount” and the disclosure set forth herein.

As used herein, the term “bioavailability” refers to the amount of a substance that is absorbed by a subject and ultimately available for biological activity in a subject's tissue and cells.

As used herein, “identifying,” refers to detecting or selecting a subject from a population of potential subjects, for example, to establish that a particular subject possesses certain properties or characteristics. “Identifying” may include, for example, self-identification, self-diagnosis, and diagnosis by a medical professional.

As used herein, the terms “preventing”, “treating”, “treatment,” “alleviating,” “ameliorating,” and the like, are used herein to generally refer to obtaining a desired pharmacological and/or physiological effect, the scopes, and meanings of which will be clear to the skilled artisan based upon the context in which these terms are used. The effect may be prophylactic in terms of preventing or partially preventing a disease, symptom, or condition thereof and/or may be therapeutic in terms of a partial or complete cure of a disease, condition, symptom, or adverse effect attributed to the disease. The term “treatment” as used herein encompasses any treatment of a disease in a mammal, particularly a human, and includes: (a) preventing the disease from occurring in a subject that may be predisposed to the disease but has not yet been diagnosed as having it; (b) inhibiting the disease or arresting its development; or (c) relieving the disease, causing regression of the disease and/or its symptoms, conditions, and co-morbidities. The scope and meaning of “preventing,” “treating,” “treatment,” “alleviating,” “ameliorating,” and “maintaining healthy levels of” would be immediately envisaged by the skilled artisan when viewing the term in the context of the disclosure and the claims.

As provided herein, the disclosure of a “ratio” of compounds and compositions corresponds to a ratio provided in terms of mass of the components present in the ratio.

When used in this disclosure, the phrase “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and can or cannot be present depending upon whether or not they affect the activity or action of the listed elements. For example, the use of a composition “consisting essentially of a composition” for the treatment of a particular disease or disorder, or the maintenance of a healthy condition, would exclude other ingredients that would materially alter the intended outcome of the composition.

As used herein, a composition that “substantially” comprises a compound means that the composition contains more than about 80% by weight, more preferably more than about 90% by weight, even more preferably more than about 95% by weight, and most preferably more than about 98% by weight of the compound.

To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

In addition, the appropriate dosage of the compositions can depend, for example, on the condition to be treated, the severity and course of the condition, whether the composition is administered for preventive or therapeutic purposes, previous therapy, the patient's clinical history and response to the composition, the type of composition used, and the discretion of the attending physician. The composition can be suitably administered to the patient at one time or over a series of treatments and may be administered to the patient at any time from diagnosis onwards. The composition may be administered as the sole treatment or in conjunction with other drugs or therapies useful in treating the condition in question.

The following exemplary embodiments are provided as illustrations of various aspects of the disclosure provided herein and are not to be construed as limiting the scope of the disclosure contained herein in any way.

### EXEMPLARY EMBODIMENTS

Exemplary Embodiments 1-91 are provided as follows:

1. An IEX-NSAID complex, wherein the IEX-NSAID complex comprises a nonsteroidal anti-inflammatory drug (NSAID) or salt thereof bound to an ion-exchange (IEX) resin.
2. The IEX-NSAID complex of embodiment 1, wherein the NSAID or salt thereof comprises naproxen or a salt thereof.
3. The IEX-NSAID complex of embodiment 2, wherein the NSAID or salt thereof comprises an alkali salt of naproxen.
4. The IEX-NSAID complex of embodiment 3, wherein the alkali salt of naproxen comprises naproxen sodium.
5. The IEX-NSAID complex of embodiment 1, wherein the IEX resin comprises an anionic exchange resin.
6. The IEX-NSAID complex of embodiment 5, wherein the anionic exchange resin comprises a cholestyramine resin.
7. The IEX-NSAID complex of embodiment 1, wherein the IEX-NSAID complex comprises about 46% NSAID or salt thereof by weight to about 52% NSAID or salt thereof by weight.
8. The IEX-NSAID complex of embodiment 1, wherein the IEX-NSAID complex releases at least 50% of bound NSAID or salt thereof within 10 minutes during a dissolution test.
9. The IEX-NSAID complex of embodiment 8, wherein the IEX-NSAID complex releases at least 80% of bound NSAID or salt thereof within 10 minutes during the dissolution test.
10. The IEX-NSAID complex of embodiment 1, wherein the IEX-NSAID complex releases at least 75% of bound NSAID or salt thereof within 30 minutes during a dissolution test.
11. The IEX-NSAID complex of embodiment 10, wherein the IEX-NSAID complex releases at least 85% of bound NSAID or salt thereof within 30 minutes during the dissolution test.
12. The IEX-NSAID complex of embodiment 1, wherein the IEX-NSAID complex releases at least 80% of bound NSAID or salt thereof within 60 minutes during a dissolution test.
13. The IEX-NSAID complex of embodiment 12, wherein the IEX-NSAID complex releases at least 90% of bound NSAID or salt thereof within 60 minutes during the dissolution test.
14. A liquid dosage form, the liquid dosage form comprising an amount of an IEX-NSAID complex, wherein the IEX-NSAID complex comprises a nonsteroidal anti-inflammatory drug (NSAID) or salt thereof bound to an ion-exchange (IEX) resin, and optionally one or more excipients selected from the group consisting of a sweetener, a thickener, a preservative, a flavorant, and any combination thereof.
15. The liquid dosage form of embodiment 14, wherein the NSAID or salt thereof comprises naproxen or a salt thereof.
16. The liquid dosage form of embodiment 15, wherein the NSAID or salt thereof comprises an alkali salt of naproxen.
17. The liquid dosage form of embodiment 16, wherein the alkali salt of naproxen comprises naproxen sodium.
18. The liquid dosage form of embodiment 14, wherein the IEX resin comprises an anionic exchange resin.
19. The liquid dosage form of embodiment 18, wherein the anionic exchange resin comprises cholestyramine.
20. The liquid dosage form of embodiment 14, wherein the liquid dosage form further comprises an amount of sucralose that is between about 0.025% wt./vol and about 1.0% wt./vol.
21. The liquid dosage form of embodiment 14, wherein the liquid dosage form further comprises an amount of glycerin that is between about 5.0% wt./vol and about 50% wt./vol.
22. The liquid dosage form of embodiment 14, wherein the liquid dosage form further comprises an amount of xanthan gum that is between about 0.05% wt./vol and about 1.0% wt./vol.
23. The liquid dosage form of embodiment 14, wherein the liquid dosage form further comprises an amount of magnesium aluminometasilicate or hydrate thereof that is between about 0.05% wt./vol. and about 0.50% wt./vol.
24. The liquid dosage form of embodiment 14, wherein the liquid dosage form further comprises an amount of one more of: (i) an amount of edetate sodium that is between about 0.05% wt./vol. and about 0.25% wt./vol., (ii) an amount of methylparaben that is between about 0.05% wt./vol. and about 0.50% wt./vol., (iii) an amount of polysorbate 80 that is between about 0.10% wt./vol. and about 0.60% wt./vol., (iv) an amount of potassium sorbate that is between about 0.05% wt./vol. and about 0.50% wt./vol., (v) an amount of propylene glycol that is between about 0.50% wt./vol. and about 10.0% wt./vol., (vi) an amount of propylparaben that is between about 0.01% wt./vol. and about 0.30% wt./vol., and (vii) an amount of sodium benzoate that is between about 0.10% wt./vol. and about 0.50% wt./vol.
25. The liquid dosage form of embodiment 14, wherein the liquid dosage form further comprises an amount of flavoring that is between about 0.10% wt./vol. and about 3.0% wt./vol.
26. The liquid dosage form of embodiment 14, wherein the liquid dosage form comprises a density, and the density is between about 1.0 g/mL and about 1.20 g/mL.
27. The liquid dosage form of embodiment 14, wherein the liquid dosage form comprises a viscosity, and the viscosity is between about 300 cP and about 600 cP.
28. The liquid dosage form of embodiment 14, wherein the liquid dosage form does not cause any irritation, burning, stinging, pricking, itching, tingling, numbness, tickling, or any combination thereof, of the oral cavity and/or pharyngeal cavity of a subject when the subject consumes the liquid dosage form.
29. The liquid dosage form of embodiment 14, wherein the liquid dosage form is adapted to be administered to a subject to treat, ameliorate, prevent, and/or reduce the severity of acute pain or a symptom thereof in the subject.
30. The liquid dosage form of embodiment 14, wherein the liquid dosage form is adapted to be administered to a subject to treat, ameliorate, prevent, and/or reduce the severity of chronic pain or a symptom thereof in the subject.
31. The liquid dosage form of embodiment 14, wherein the liquid dosage form is adapted to be administered to a subject to treat, ameliorate, prevent, and/or reduce the severity of acute inflammation or a symptom thereof in the subject.
32. The liquid dosage form of embodiment 14, wherein the liquid dosage form is adapted to be administered to a subject to treat, ameliorate, prevent, and/or reduce the severity of chronic inflammation or a symptom thereof in the subject.
33. The liquid dosage form of embodiment 14, wherein the liquid dosage form is adapted to be administered to a subject to treat, ameliorate, prevent, and/or reduce the severity of a fever or a symptom thereof in the subject.
34. The liquid dosage form of embodiment 14, wherein the liquid dosage form is adapted to be administered to a subject to treat, ameliorate, prevent, and/or reduce the severity of blood clots or a symptom thereof in the subject.
35. The liquid dosage form of embodiment 14, wherein the liquid dosage form is adapted to be administered to a subject to treat, ameliorate, prevent, and/or reduce the severity of a heart attack or a symptom thereof in the subject.
36. The liquid dosage form of embodiment 14, wherein the liquid dosage form is adapted to be administered to a subject to treat, ameliorate, prevent, and/or reduce the severity of a stroke or a symptom thereof in the subject.
37. The liquid dosage form of embodiment 14, wherein the liquid dosage form is adapted to be administered to a subject to treat, ameliorate, prevent, and/or reduce the severity of osteoarthritis or a symptom thereof in the subject.
38. The liquid dosage form of embodiment 14, wherein the liquid dosage form is adapted to be administered to a subject to treat, ameliorate, prevent, and/or reduce the severity of rheumatoid arthritis or a symptom thereof in the subject.
39. The liquid dosage form of embodiment 14, wherein the liquid dosage form is adapted to be administered to a subject to treat, ameliorate, prevent, and/or reduce the severity of headaches or a symptom thereof in the subject.
40. The liquid dosage form of embodiment 14, wherein the liquid dosage form is adapted to be administered to a subject to treat, ameliorate, prevent, and/or reduce the severity of migraines or a symptom thereof in the subject.
41. The liquid dosage form of embodiment 14, wherein the liquid dosage form is adapted to be administered to a subject to treat, ameliorate, prevent, and/or reduce the severity of gout or a symptom thereof in the subject.
42. The liquid dosage form of embodiment 14, wherein the liquid dosage form is adapted to be administered to a subject to treat, ameliorate, prevent, and/or reduce the severity of dysmenorrhea or a symptom thereof in the subject.
43. The liquid dosage form of embodiment 14, wherein the liquid dosage form is adapted to be administered to a subject to treat, ameliorate, prevent, and/or reduce the severity of muscle stiffness or a symptom thereof in the subject.
44. The liquid dosage form of embodiment 14, wherein the liquid dosage form is adapted to be administered to a subject to treat, ameliorate, prevent, and/or reduce the severity of ileus or a symptom thereof in the subject.
45. The liquid dosage form of embodiment 14, wherein the liquid dosage form is adapted to be administered to a subject to treat, ameliorate, prevent, and/or reduce the severity of macular edema or a symptom thereof in the subject.
46. The liquid dosage form of embodiment 14, wherein the liquid dosage form is adapted to be administered to a subject to temporarily relieve minor aches and pains due to arthritis.
47. The liquid dosage form of embodiment 14, wherein the liquid dosage form is adapted to be administered to a subject to temporarily relieve minor aches and pains due to muscular aches.
48. The liquid dosage form of embodiment 14, wherein the liquid dosage form is adapted to be administered to a subject to temporarily relieve minor aches and pains due to backache.
49. The liquid dosage form of embodiment 14, wherein the liquid dosage form is adapted to be administered to a subject to temporarily relieve minor aches and pains due to menstrual cramps.
50. The liquid dosage form of embodiment 14, wherein the liquid dosage form is adapted to be administered to a subject to temporarily relieve minor aches and pains due to headache.
51. The liquid dosage form of embodiment 14, wherein the liquid dosage form is adapted to be administered to a subject to temporarily relieve minor aches and pains due to toothache.
52. The liquid dosage form of embodiment 14, wherein the liquid dosage form is adapted to be administered to a subject to temporarily relieve minor aches and pains due to the common cold.
53. The liquid dosage form of embodiment 14, wherein the liquid dosage form is adapted to be administered to a subject to temporarily reduce fever.
54. The liquid dosage form of embodiment 14, wherein the IEX-NSAID complex releases at least 50% of bound NSAID or salt thereof within 10 minutes during a dissolution test.
55. The liquid dosage form of embodiment 54, wherein the IEX-NSAID complex releases at least 80% of bound NSAID or salt thereof within 10 minutes during a dissolution test.
56. The liquid dosage form of embodiment 14, wherein the IEX-NSAID complex releases at least 75% of bound NSAID or salt thereof within 30 minutes during a dissolution test.
57. The liquid dosage form of embodiment 56, wherein the IEX-NSAID complex releases at least 85% of bound NSAID or salt thereof within 30 minutes during a dissolution test.
58. The liquid dosage form of embodiment 14, wherein the IEX-NSAID complex releases at least 80% of bound NSAID or salt thereof within 60 minutes during a dissolution test.
59. The liquid dosage form of embodiment 58, wherein the IEX-NSAID complex releases at least 90% of bound NSAID or salt thereof within 60 minutes during a dissolution test.
60. A method of making an IEX-NSAID complex, the method comprising:
(i) providing an amount of a nonsteroidal anti-inflammatory drug (NSAID) or salt thereof in an aqueous solution;
(ii) providing an amount of an ion-exchange (IEX) resin in the aqueous solution;
(iii) optionally, adjusting the pH of the aqueous solution to a binding pH;
(iv) mixing the aqueous solution at a mixing temperature for an amount of mixing time;
(v) collecting a precipitant after the amount mixing time has elapsed;
(vi) washing the precipitant with a volume aqueous washing solution; and
(vii) dying the precipitant at a drying temperature for an amount of drying time.
   61. The method of embodiment 60, wherein the NSAID or salt thereof comprises naproxen or a salt thereof.
   62. The method of embodiment 61, wherein the NSAID or salt thereof comprises naproxen sodium.
   63. The method of embodiment 60, wherein the IEX resin comprises an anionic exchange resin.
   64. The method of embodiment 63, wherein the anionic exchange resin comprises a cholestyramine resin.
   65. The method of embodiment 60, wherein the amount of NSAID or salt thereof and the amount of IEX resin define a ratio, and the ratio is between about 1:0.5 by mass and about 1:4 by mass (NSAID or salt thereof to IEX resin).
   66. The method of embodiment 65, wherein the ratio is between about 1:1.25 by mass and about 1:1.5 by mass (NSAID or salt thereof to IEX resin).
   67. The method of embodiment 60, wherein the mixing temperature is between about 20° C. and about 25° C.
   68. The method of embodiment 60, wherein the mixing time is between about 0.5 hrs and about 6 hrs.
   69. The method of embodiment 60, wherein the method comprises a binding efficiency, wherein the binding efficiency is given by a percentage of NSAID or salt thereof bound to the IEX resin compared to the amount of NSAID or salt thereof provided in the aqueous solution, and wherein the binding efficiency is at least about 85%.
   70. The method of embodiment 69, wherein the binding efficiency is at least about 95%.
   71. A method of making a liquid dosage form comprising an IEX-NSAID complex, the method comprising:
(i) providing an amount of a nonsteroidal anti-inflammatory drug (NSAID) or salt thereof in an aqueous solution;
(ii) providing an amount of an ion-exchange (IEX) resin in the aqueous solution;
(iii) providing an amount of an amount of one or more sweeteners in the aqueous solution;
(iii) providing an amount of an amount of one or more thickeners in the aqueous solution;
(iv) providing an amount of an amount of one or more preservatives in the aqueous solution;
(v) providing an amount of an amount of a flavorant in the aqueous solution;
(vi) optionally, adjusting the pH of the aqueous solution to a binding pH; and
(vii) mixing the aqueous solution at a mixing temperature for an amount of total mixing time.
   72. The method of embodiment 71, wherein the amount of NSAID or salt thereof is between about 1.0% wt./vol. and about 5.0 wt./vol.
   73. The method of embodiment 72, wherein the NSAID or salt thereof comprises naproxen or a salt thereof.
   74. The method of embodiment 73, wherein the NSAID or salt thereof comprises naproxen sodium.
   75. The method of embodiment 71, wherein the amount of IEX resin is between about 1.0% wt./vol. and about 5.0 wt./vol.
   76. The method of embodiment 75, wherein the IEX resin comprises an anionic exchange resin.
   77. The method of embodiment 76, wherein the anionic exchange resin comprises a cholestyramine resin.
   78. The method of embodiment 71, wherein the amount of NSAID or salt thereof and the amount of IEX resin define a ratio, and the ratio is between about 1:0.5 by mass and about 1:4 by mass (NSAID or salt thereof to IEX resin).
   79. The method of embodiment 78, wherein the ratio is between about 1:1.25 by mass and about 1:1.5 by mass (NSAID or salt thereof to IEX resin).
   80. The method of embodiment 71, wherein the one or more sweeteners comprises sucralose.
   81. The method of embodiment 80, wherein the amount of sucralose is between about 0.025% wt./vol and about 1.0% wt./vol.
   82. The method of embodiment 71, wherein the one or more thickeners comprise glycerin, xanthan gum, and magnesium aluminometasilicate or hydrate thereof.
   83. The method of embodiment 82, wherein the amount of glycerin is between about 5.0% wt./vol and about 50% wt./vol., the amount of xanthan gum is between about 0.05% wt./vol and about 1.0% wt./vol., and the amount of magnesium aluminometasilicate or hydrate thereof is between about 0.05% wt./vol. and about 0.50% wt./vol.
   84. The method of embodiment 71, wherein the one or more preservatives comprises one or more of: (i) an amount of edetate sodium that is between about 0.05% wt./vol. and about 0.25% wt./vol., (ii) an amount of methylparaben that is between about 0.05% wt./vol. and about 0.50% wt./vol., (iii) an amount of polysorbate 80 that is between about 0.10% wt./vol. and about 0.60% wt./vol., (iv) an amount of potassium sorbate that is between about 0.05% wt./vol. and about 0.50% wt./vol., (v) an amount of propylene glycol that is between about 0.50% wt./vol. and about 10.0% wt./vol., (vi) an amount of propylparaben that is between about 0.01% wt./vol. and about 0.30% wt./vol., and (vii) an amount of sodium benzoate that is between about 0.10% wt./vol. and about 0.50% wt./vol.
   85. The method of embodiment 71, wherein the flavorant is grape flavoring.
   86. The method of embodiment 71, wherein the amount of flavorant is between about 0.10% wt./vol. and about 3.0% wt./vol.
   87. The method of embodiment 71, wherein the mixing temperature is between about 20° C. and about 25° C.
   88. The method of embodiment 71, wherein the total mixing time is between about 0.5 hrs and about 10 hrs.
   89. The method of embodiment 71, wherein the method comprises a binding efficiency, wherein the binding efficiency is given by a percentage of NSAID or salt thereof bound to the IEX resin compared to the amount of NSAID or salt thereof provided in the aqueous solution, and wherein the binding efficiency is at least about 85%.
   90. The method of embodiment 89, wherein the binding efficiency is at least about 95%.
   91. The method of embodiment 71, wherein the method comprises an unbound content, wherein the unbound content is given by a concentration of NSAID or salt thereof remaining in the aqueous solution after the mixing time has elapsed, and wherein the unbound content is less than about 2 mg/mL.

### EXAMPLES

### Example 1

An initial set of experiments was performed to determine an appropriate IEX resin that could produce IEX resin-bound naproxen sodium particles that could be incorporated in a liquid dosage form, such that the resulting liquid dosage form (i) could be feasibly administered to a subject, e.g., possessed an appropriate viscosity, mouth-feel, etc., and (ii) lacked any undesirable chemesthetic effects characteristic of naproxen sodium when ingested, e.g., irritation, burning, stinging, pricking, itching, tingling, numbness, tickling, etc. of the oral cavity and/or pharyngeal cavity.

Initially, Eudragit, a cationic copolymer based on dimethylaminoethyl methacrylate, butyl methacrylate, and methyl methacrylate, was investigated as an IEX resin. Eudragit was reacted with naproxen sodium via wet granulation followed by fluid bed drying, to generate Eudragit-coated naproxen sodium particles. To evaluate feasibility of a liquid dosage form, the Eudragit-coated naproxen sodium particles were provided in a xanthan gum suspension matrix, which resulted in a gelatinous mass that was not suitable as a liquid dosage form, and Eudragit was abandoned as an IEX resin.

A second IEX resin, cholestyramine resin, a type I anionic IEX resin based on crosslinked gel polystyrene, was investigated. Cholestyramine resin was reacted with naproxen sodium via wet granulation followed by fluid bed drying, to generate cholestyramine resin-bound naproxen sodium particles. To evaluate feasibility of a liquid dosage form, the cholestyramine resin-bound naproxen sodium particles were provided in a xanthan gum suspension matrix, which resulted in a liquid dosage form lacking undesirable chemesthetic effects; however this liquid dosage form had an undesirable mouth feel arising from cholestyramine resin-bound naproxen sodium particles being too large.

Because particle size was a critical parameter in attaining a desirable mouth feel, a batch of cholestyramine resin-bound naproxen sodium particles were dry milled in a pestle and mortar to reduce their particle size to sub-150 μm. The dry milled cholestyramine resin-bound naproxen sodium particles were then incorporated into a xanthan gum suspension matrix. Upon doing so, the resulting liquid dosage form lacked chemesthetic effects and had a desirable mouth feel. However, because manually dry milling cholestyramine resin-bound naproxen sodium particles was not feasible for large scale manufacturing, a final experiment was performed to produce cholestyramine resin-bound naproxen sodium particles of an appropriate size to attain a desirable mouth feel.

250 g of naproxen sodium was dissolved in 2 L of water. 250 g of cholestyramine resin was added to the naproxen sodium solution. The solution was mixed for 4 hrs at room temperature, after which a precipitant was formed. The precipitant was collected and washed twice with 1 L of water per wash. The precipitant was subsequently collected in dried in an oven at 75° C. for 6 hrs. The dried precipitant was analyzed by high-performance liquid chromatography and found to be cholestyramine resin-bound naproxen sodium comprising 46-52% naproxen sodium by weight. The cholestyramine resin-bound naproxen sodium particles herein were identified as “Naproxen-Resin Complex API Batch” and stored for subsequent analysis.

### Example 2

In addition to developing Naproxen-Resin Complex API Batch particles, the present inventors also sought to develop a method for making cholestyramine resin-bound naproxen sodium particles that formed directly in a liquid dosage form during its manufacturing process, or in situ, to streamline the manufacturing process of a naproxen sodium liquid dosage form.

Critically, the present inventors identified that unlike the Naproxen-Resin Complex API Batch particles, which could be washed to remove unbound naproxen sodium, cholestyramine resin-bound naproxen sodium particles formed in situ could not be washed to remove any unbound naproxen sodium and thus any unbound naproxen sodium would remain within the liquid dosage form.

To determine the permissible concentration of unbound naproxen sodium that induced chemesthetic effects, two experiments were performed.

In the first experiment, four aqueous solutions containing 0.5 mg/mL, 1 mg/mL, 2 mg/mL, and 3 mg/mL of naproxen sodium were prepared and administered to human subjects. Among these concentrations, chemesthetic effects were only observed in subjects administered the 3 mg/mL naproxen sodium concentration.

In second experiment, four aqueous suspensions containing 2 mg/mL 2.5 mg/mL, 3 mg/mL, and 5 mg/mL of naproxen sodium, as well as constant amounts of excipients, e.g., thickeners, flavorants, and sweeteners, were prepared and administered to human subjects. Chemesthetic effects were observed for all aqueous suspensions, with the lowest frequency (25%) occurring at the 2 mg/mL naproxen sodium concentration.

From these experiments, the present inventors sought to optimize the reaction parameters for making in situ cholestyramine resin-bound naproxen sodium particles to ensure an unbound naproxen sodium of less than 2 mg/mL was obtained.

### Example 3

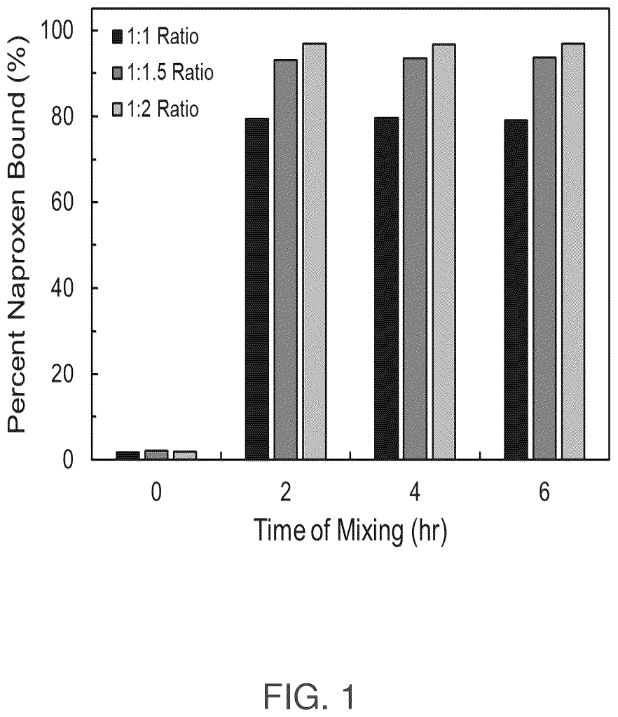

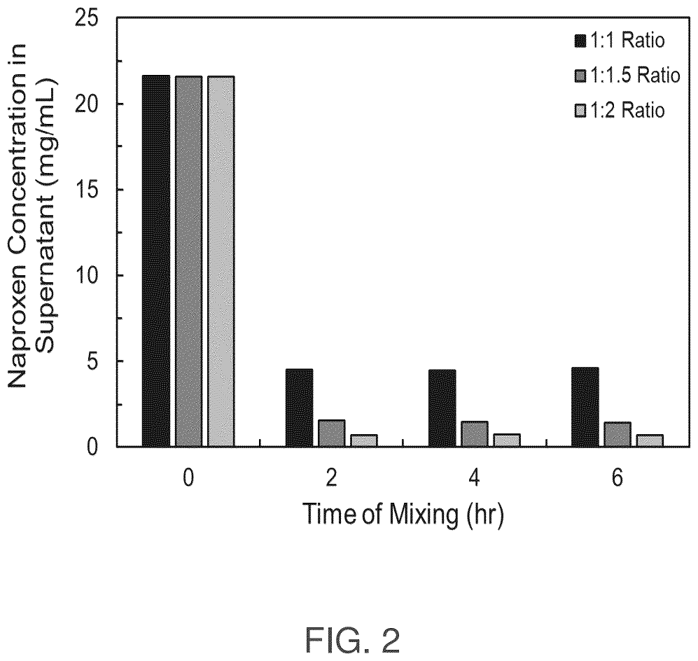

Three solutions containing 11 g of naproxen sodium in 500 g of water were prepared. An amount of cholestyramine resin—11 g (“1:1 Ratio”), 17 g (“1:1.5 Ratio”), or 22 g (“1:2 Ratio”)—was added to each of the three naproxen sodium solutions. The solutions were mixed for 6 hrs at room temperature. During the mixing step, 10 mL aliquots were withdrawn at 2, 4, and 6 hrs for analysis. The results are shown in FIGS. 1 and 2.

FIG. 1 shows the percentage of naproxen sodium bound by cholestyramine resin in an aqueous solution following a mixing reaction with cholestyramine resin as a function of (i) the ratio of naproxen sodium to cholestyramine resin (mass to mass) in the aqueous solution, and (ii) duration of the mixing reaction (“Time of Mixing”). FIG. 2 shows the amount of naproxen sodium that remained in an aqueous solution, i.e., unbound, following a mixing reaction with cholestyramine resin as a function of (i) the ratio of naproxen sodium to cholestyramine resin (mass to mass) in the aqueous solution, and (ii) duration of the mixing reaction (“Time of Mixing”).

At a 1:1 ratio of naproxen sodium to cholestyramine resin, between 79.0% and 79.5% of naproxen sodium was bound during the 2-6 hr reaction period (FIG. 1—“1:1 Ratio”), corresponding to between 4.49 mg/mL and 4.61 mg/mL of unbound naproxen sodium in solution (FIG. 2—“1:1 Ratio”).

At a 1:1.5 ratio of naproxen sodium to cholestyramine resin, between 93.0% and 93.6% of naproxen sodium was bound during the 2-6 hr reaction period (FIG. 1—“1:1.5 Ratio”), corresponding to between 1.41 mg/mL and 1.53 mg/mL of unbound naproxen sodium in solution (FIG. 2—“1:1.5 Ratio”).

At a 1:2 ratio of naproxen sodium to cholestyramine resin, between 96.7% and 96.9% of naproxen sodium was bound during the 2-6 hr reaction period (FIG. 1—“1:2 Ratio”), corresponding to between 0.69 mg/mL and 0.72 mg/mL of unbound naproxen sodium in solution (FIG. 2—“1:2 Ratio”).

### Example 4

Example 3 demonstrated that the mixing reaction yields insignificant differences after two hours and that adding a larger amount of cholestyramine resin improves the binding of naproxen sodium, thereby reducing the amount of unbound naproxen sodium that remains in solution. Accordingly, an additional experiment was undertaken to evaluate the effect of shorter mixing times, i.e., 1-4 hrs, and different ratios of naproxen sodium to cholestyramine resin, i.e., 1:0.5 to 1:2, on naproxen sodium binding, in a more concentrated aqueous solution of naproxen sodium.

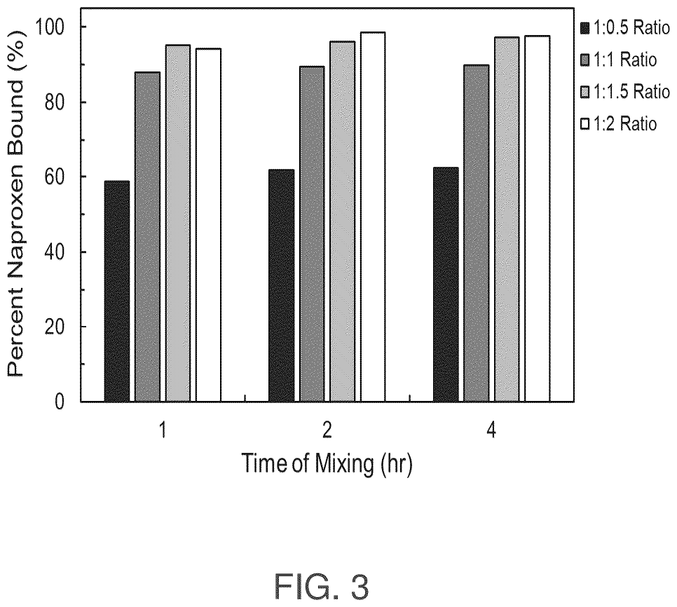

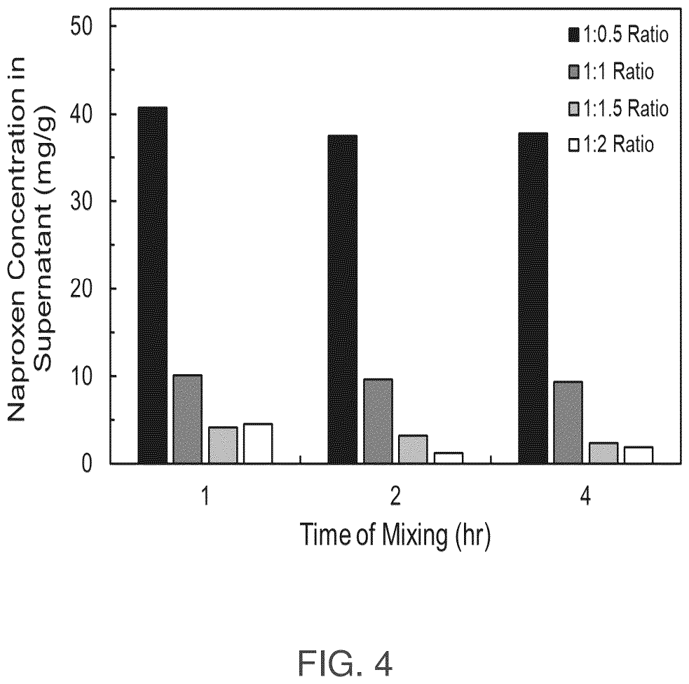

Four solutions containing 11 g of naproxen sodium in 100 g of water were prepared. An amount of cholestyramine resin—5.5 g (“1:0.5 Ratio”), 11 g (“1:1 Ratio”), 17 g (“1:1.5 Ratio”), or 22 g (“1:2 Ratio”)—was added to each of the four naproxen sodium solutions. The solutions were mixed for 4 hrs at room temperature. During the mixing step, 2.5 g aliquots were withdrawn at 1, 2, and 4 hrs for analysis. The results are shown in FIGS. 3 and 4.

FIG. 3 shows the percentage of naproxen sodium bound by cholestyramine resin in an aqueous solution following a mixing reaction with cholestyramine resin as a function of (i) the ratio of naproxen sodium to cholestyramine resin (mass to mass) in the aqueous solution, and (ii) duration of the mixing reaction (“Time of Mixing”). FIG. 4 shows the amount of naproxen sodium that remained in an aqueous solution, i.e., unbound, following a mixing reaction with cholestyramine resin as a function of (i) the ratio of naproxen sodium to cholestyramine resin (mass to mass) in the aqueous solution, and (ii) duration of the mixing reaction (“Time of Mixing”).

At a 1:0.5 ratio of naproxen sodium to cholestyramine resin, between 58.7% and 62.3% of naproxen sodium was bound during the 1-4 hr reaction period (FIG. 3—“1:0.5 Ratio”), corresponding to between 37.4 mg/mL and 40.7 mg/mL of unbound naproxen sodium in solution (FIG. 4—“1:0.5 Ratio”).

At a 1:1 ratio of naproxen sodium to cholestyramine resin, between 88.0% and 89.9% of naproxen sodium was bound during the 1-4 hr reaction period (FIG. 3—“1:1 Ratio”), corresponding to between 9.4 mg/mL and 10.1 mg/mL of unbound naproxen sodium in solution (FIG. 4—“1:1 Ratio”).

At a 1:1.5 ratio of naproxen sodium to cholestyramine resin, between 95.1% and 97.3% of naproxen sodium was bound during the 1-4 hr reaction period (FIG. 3—“1:1.5 Ratio”), corresponding to between 2.3 mg/mL and 4.1 mg/mL of unbound naproxen sodium in solution (FIG. 4—“1:1.5 Ratio”).

At a 1:2 ratio of naproxen sodium to cholestyramine resin, between 94.2% and 98.5% of naproxen sodium was bound during the 1-4 hr reaction period (FIG. 3—“1:2 Ratio”), corresponding to between 1.2 mg/mL and 4.6 mg/mL of unbound naproxen sodium in solution (FIG. 4—“1:2 Ratio”).

### Example 5

Example 4 demonstrated that the mixing reaction yields insignificant differences after one hour and that adding a larger amount of cholestyramine resin improves the binding of naproxen sodium, thereby reducing the amount of unbound naproxen sodium that remains in solution. At higher concentrations of cholestyramine resin, i.e., 1:2 ratio, the viscosity of the solution became large and there was difficulty separating the formed precipitant from the supernatant.

Accordingly, an additional experiment was undertaken to evaluate the effect of shorter mixing times, i.e., 0.5-2 hrs, and different ratios of naproxen sodium to cholestyramine resin, i.e., 1:1.25 to 1:1.5, on naproxen sodium binding, in an equally concentrated aqueous solution of naproxen sodium.

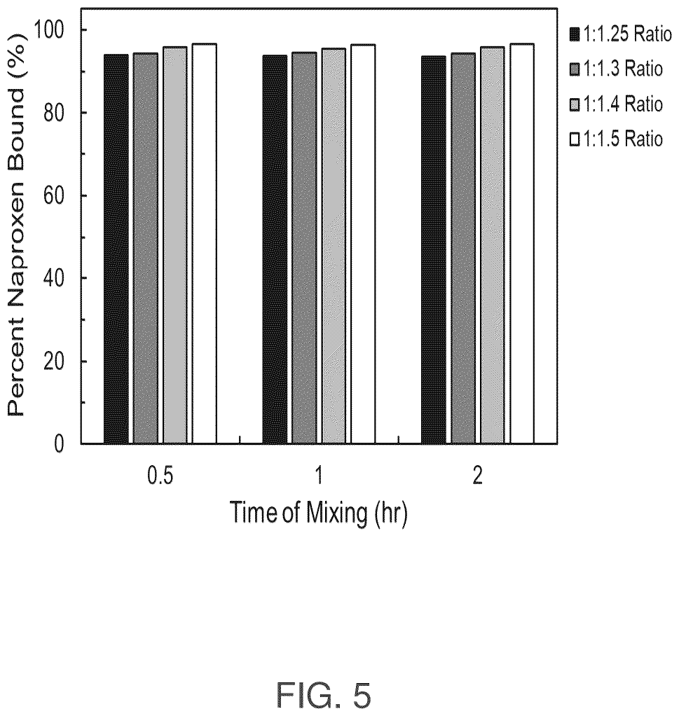

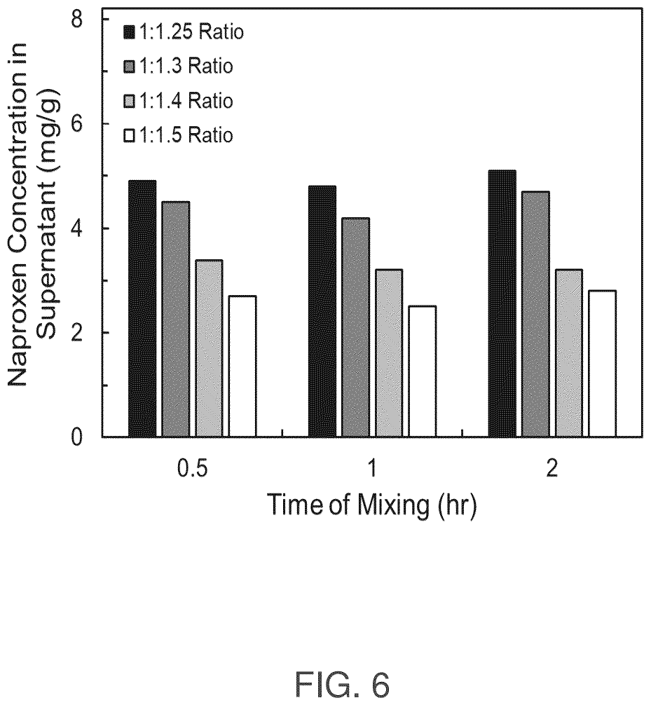

Four solutions containing 11 g of naproxen sodium in 100 g of water were prepared. An amount of cholestyramine resin—13.75 g (“1:1.25 Ratio”), 14.3 g (“1:1.3 Ratio”), 15.4 g (“1:1.4 Ratio”), or 16.5 g (“1:1.5 Ratio”)—was added to each of the four naproxen sodium solutions. The solutions were mixed for 2 hrs at room temperature. During the mixing step, 10 g aliquots were withdrawn at 0.5, 1, and 2 hrs for analysis. The results are shown in FIGS. 5 and 6.

FIG. 5 shows the percentage of naproxen sodium bound by cholestyramine resin in an aqueous solution following a mixing reaction with cholestyramine resin as a function of (i) the ratio of naproxen sodium to cholestyramine resin (mass to mass) in the aqueous solution, and (ii) duration of the mixing reaction (“Time of Mixing”). FIG. 6 shows the amount of naproxen sodium that remained in an aqueous solution, i.e., unbound, following a mixing reaction with cholestyramine resin as a function of (i) the ratio of naproxen sodium to cholestyramine resin (mass to mass) in the aqueous solution, and (ii) duration of the mixing reaction (“Time of Mixing”).

At a 1:1.25 ratio of naproxen sodium to cholestyramine resin, between 93.6% and 93.9% of naproxen sodium was bound during the 0.5-2 hr reaction period (FIG. 5—“1:1.25 Ratio”), corresponding to between 4.8 mg/mL and 5.1 mg/mL of unbound naproxen sodium in solution (FIG. 6—“1:1.25 Ratio”).

At a 1:1.3 ratio of naproxen sodium to cholestyramine resin, between 94.3% and 94.4% of naproxen sodium was bound during the 0.5-2 hr reaction period (FIG. 5—“1:1.3 Ratio”), corresponding to between 4.2 mg/mL and 4.7 mg/mL of unbound naproxen sodium in solution (FIG. 6—“1:1.3 Ratio”).

At a 1:1.4 ratio of naproxen sodium to cholestyramine resin, between 95.5% and 95.8% of naproxen sodium was bound during the 0.5-2 hr reaction period (FIG. 5—“1:1.4 Ratio”), corresponding to between 3.2 mg/mL and 3.4 mg/mL of unbound naproxen sodium in solution (FIG. 6—“1:1.4 Ratio”).

At a 1:1.5 ratio of naproxen sodium to cholestyramine resin, between 96.4% and 96.6% of naproxen sodium was bound during the 0.5-2 hr reaction period (FIG. 5—“1:1.5 Ratio”), corresponding to between 2.5 mg/mL and 2.8 mg/mL of unbound naproxen sodium in solution (FIG. 6—“1:1.5 Ratio”).

### Example 6

Example 5 demonstrated that the mixing reaction yields insignificant differences after a half hour and that adding a larger amount of cholestyramine resin improves the binding of naproxen sodium, thereby reducing the amount of unbound naproxen sodium that remains in solution. As was the case in Example 4, at higher concentrations of cholestyramine resin, i.e., 1:1.5 Ratio, the viscosity of the solution became large and there was difficulty separating the formed precipitant from the supernatant. In light of the results from Examples 3-5, an additional experiment was undertaken to evaluate the effect of a wide range of ratios of naproxen sodium to cholestyramine resin, i.e., 1:0 to 1:4, on naproxen sodium binding, in less concentrated aqueous solutions of naproxen sodium, 1.1 mg/mL or 2.2 mg/mL.

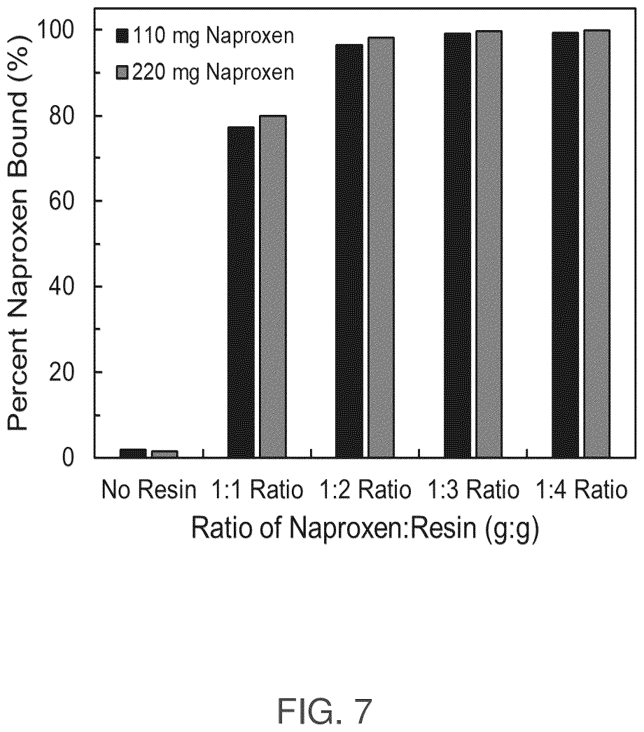

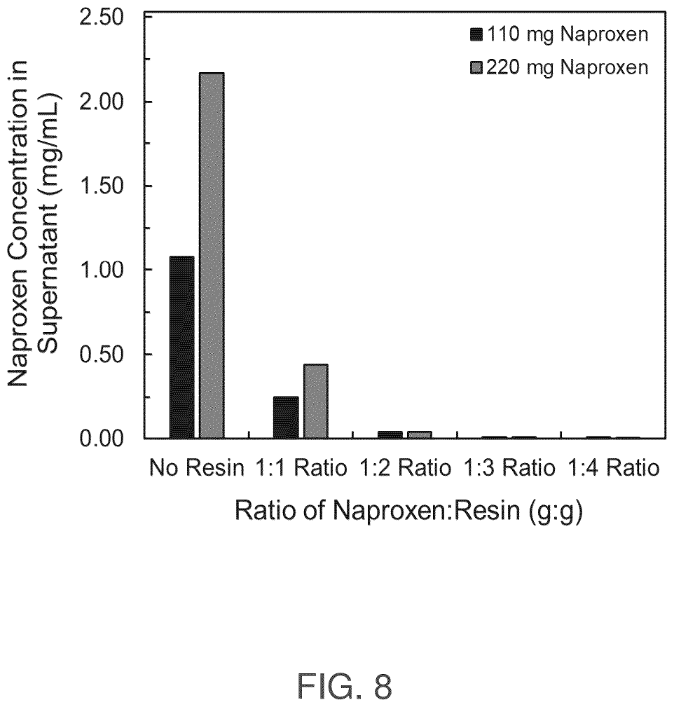

Five solutions containing 110 mg of naproxen sodium in 50 mL of water were prepared. An amount of cholestyramine resin—0 mg (“No Resin”), 110 mg (“1:1 Ratio”), 220 mg (“1:2 Ratio”), 330 mg (“1:3 Ratio”), or 440 mg (“1:4 Ratio”)—was added to each of the five naproxen sodium solutions, and an additional 50 mL of water was added. Additionally, five solutions containing 220 mg of naproxen sodium in 50 mL of water were prepared. An amount of cholestyramine resin—0 mg (“No Resin”), 110 mg (“1:1 Ratio”), 220 mg (“1:2 Ratio”), 330 mg (“1:3 Ratio”), or 440 mg (“1:4 Ratio”)—was added to each of the five naproxen sodium solutions, and an additional 50 mL of water was added. The solutions were mixed for 1 hr at room temperature, after which analysis was performed. The results are shown in FIGS. 7 and 8.

FIG. 7 shows the percentage of naproxen sodium bound by cholestyramine resin in an aqueous solution following a mixing reaction with cholestyramine resin as a function of (i) the ratio of naproxen sodium to cholestyramine resin (mass to mass) in the aqueous solution, and (ii) initial naproxen sodium concentration in the aqueous solution. FIG. 8 shows the amount of naproxen sodium that remained in an aqueous solution, i.e., unbound, following a mixing reaction with cholestyramine resin as a function of (i) the ratio of naproxen sodium to cholestyramine resin (mass to mass) in the aqueous solution, and (ii) initial naproxen sodium concentration in the aqueous solution.

For solutions initially containing 110 mg naproxen sodium (1.1 mg/mL), with no cholestyramine resin added, 1.08 mg/mL of unbound naproxen sodium remained in solution following the reaction period (FIG. 8—“No Resin, 110 mg Naproxen”).

At a 1:1 ratio of naproxen sodium to cholestyramine resin, 77.3% of naproxen sodium was bound following the reaction period (FIG. 7—“1:1 Ratio, 110 mg Naproxen”), corresponding to 0.250 mg/mL of unbound naproxen sodium in solution (FIG. 8—“1:1 Ratio, 110 mg Naproxen”).

At a 1:2 ratio of naproxen sodium to cholestyramine resin, 96.4% of naproxen sodium was bound following the reaction period (FIG. 7—“1:2 Ratio, 110 mg Naproxen”), corresponding to 0.040 mg/mL of unbound naproxen sodium in solution (FIG. 8—“1:2 Ratio, 110 mg Naproxen”).

At a 1:3 ratio of naproxen sodium to cholestyramine resin, 99.1% of naproxen sodium was bound following the reaction period (FIG. 7—“1:3 Ratio, 110 mg Naproxen”), corresponding to 0.010 mg/mL of unbound naproxen sodium in solution (FIG. 8—“1:3 Ratio, 110 mg Naproxen”).

At a 1:4 ratio of naproxen sodium to cholestyramine resin, 99.3% of naproxen sodium was bound following the reaction period (FIG. 7—“1:4 Ratio, 110 mg Naproxen”), corresponding to 0.008 mg/mL of unbound naproxen sodium in solution (FIG. 8—“1:4 Ratio, 110 mg Naproxen”).

For solutions initially containing 220 mg naproxen sodium (2.2 mg/mL), with no cholestyramine added, 2.17 mg/mL of unbound naproxen sodium remained in solution following the reaction period (FIG. 8—“No Resin, 220 mg Naproxen”).

At a 1:1 ratio of naproxen sodium to cholestyramine resin, 80.0% of naproxen sodium was bound following the reaction period (FIG. 7—“1:1 Ratio, 220 mg Naproxen”), corresponding to 0.440 mg/mL of unbound naproxen sodium in solution (FIG. 8—“1:1 Ratio, 220 mg Naproxen”).

At a 1:2 ratio of naproxen sodium to cholestyramine resin, 98.2% of naproxen sodium was bound following the reaction period (FIG. 7—“1:2 Ratio, 220 mg Naproxen”), corresponding to 0.040 mg/mL of unbound naproxen sodium in solution (FIG. 8—“1:2 Ratio, 220 mg Naproxen”).

At a 1:3 ratio of naproxen sodium to cholestyramine resin, 99.6% of naproxen sodium was bound following the reaction period (FIG. 7—“1:3 Ratio, 220 mg Naproxen”), corresponding to 0.009 mg/mL of unbound naproxen sodium in solution (FIG. 8—“1:3 Ratio, 220 mg Naproxen”).

At a 1:4 ratio of naproxen sodium to cholestyramine resin, 99.8% of naproxen sodium was bound following the reaction period (FIG. 7—“1:4 Ratio, 220 mg Naproxen”), corresponding to 0.004 mg/mL of unbound naproxen sodium in solution (FIG. 8—“1:4 Ratio, 220 mg Naproxen”).

### Example 7

Example 6 demonstrated that a large amount of naproxen sodium binding can be achieved, thereby reducing the amount of unbound naproxen sodium that remains in solution. At higher concentrations of cholestyramine resin, i.e., 1:3 Ratio and 1:4 Ratio, the viscosity of the solution became large as with Examples 4-6. Moreover, the concentration used in Example 6 was too low for the target concentration in the finished product, i.e., 22 mg/mL. Accordingly, an additional experiment was undertaken to evaluate the effect of a range of ratios of naproxen sodium to cholestyramine resin, i.e., 1:1.25 to 1:2, on naproxen sodium binding, for a concentration level of the finished product (22 mg/mL), the concentration as 50% final batch volume (44 mg/mL), and the concentration at 25% batch volume (88 mg/mL) to determine how much water would be needed during the in situ binding step of batch manufacturing.

Four solutions containing 2.2 g of naproxen sodium in 85 mL of water were prepared. An amount of cholestyramine resin—2.75 g (“1:1.25 Ratio”), 3.3 g (“1:1.5 Ratio”), 3.85 g (“1:1.75 Ratio”), or 4.4 g (“1:2 Ratio”)—was added to each of the four naproxen sodium solutions, and an additional 15 mL of water was added.

Additionally, four solutions containing 4.4 g of naproxen sodium in 85 mL of water were prepared. An amount of cholestyramine resin—5.5 g (“1:1.25 Ratio”), 6.6 g (“1:1.5 Ratio”), 7.7 g (“1:1.75 Ratio”), or 8.8 g (“1:2 Ratio”)—was added to each of the four naproxen sodium solutions, and an additional 15 mL of water was added.

Further, four solutions containing 8.8 g of naproxen sodium in 85 mL of water were prepared. An amount of cholestyramine resin—11.0 g (“1:1.25 Ratio”), 13.2 g (“1:1.5 Ratio”), 15.4 g (“1:1.75 Ratio”), or 17.6 g (“1:2 Ratio”)—was added to each of the four naproxen sodium solutions, and an additional 15 mL of water was added.

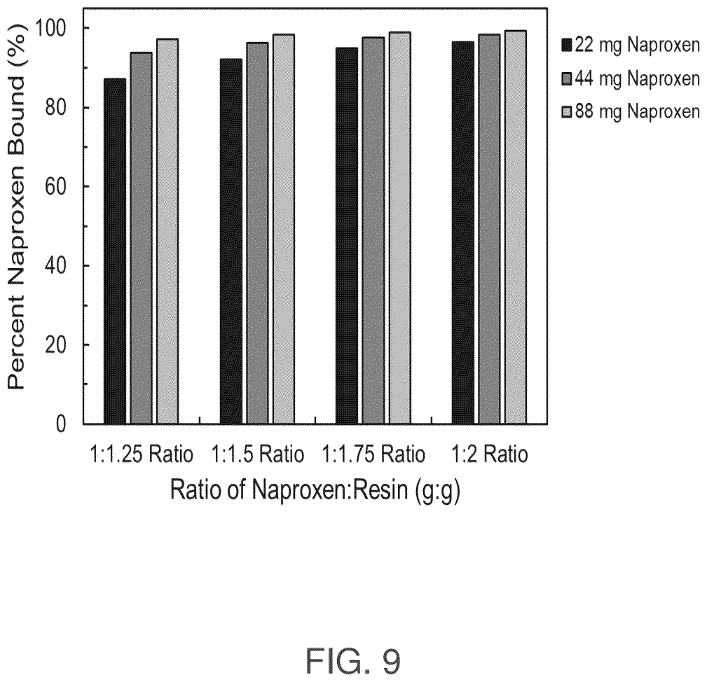

All of the aforementioned solutions were mixed for 0.5 hrs at room temperature, after which analysis was performed. The results are shown in FIGS. 9-11.

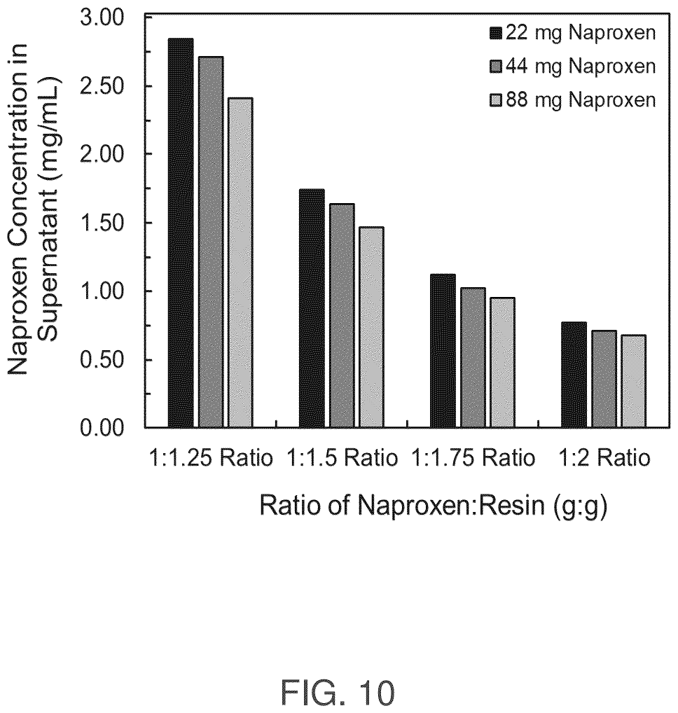

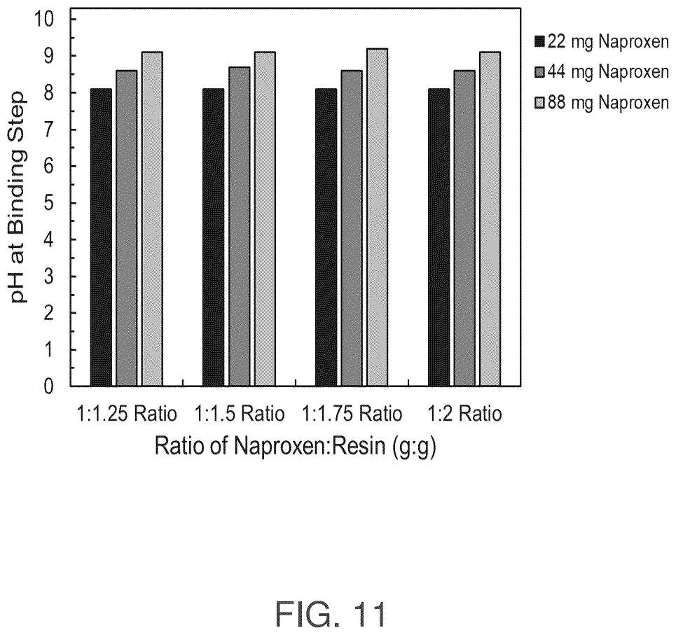

FIG. 9 shows the percentage of naproxen sodium bound by cholestyramine resin in an aqueous solution following a mixing reaction with cholestyramine resin as a function of (i) the ratio of naproxen sodium to cholestyramine resin (mass to mass) in the aqueous solution, and (ii) initial naproxen sodium concentration in the aqueous solution. FIG. 10 shows the amount of naproxen sodium that remained in an aqueous solution, i.e., unbound, following a mixing reaction with cholestyramine resin as a function of (i) the ratio of naproxen sodium to cholestyramine resin (mass to mass) in the aqueous solution, and (ii) initial naproxen sodium concentration in the aqueous solution. FIG. 11 shows the pH of the solution upon addition of cholestyramine resin as a function of (i) the ratio of naproxen sodium to cholestyramine resin (mass to mass) in the aqueous solution, and (ii) initial naproxen sodium concentration in the aqueous solution. Notably, the optimal pH for the cholestyramine resin to bind to the naproxen is above pH 7.5. (see Calvo et al. “Interaction of naproxen with cholestyramine,” Biopharm. Drug Dispos., 5, pp. 33-42., the contents of which is hereby incorporated by reference in their entirety).

For solutions initially containing 2.2 g of naproxen sodium (22 mg/mL), at a 1:1.25 ratio of naproxen sodium to cholestyramine resin, 87.1% of naproxen sodium was bound following the reaction period (FIG. 9—“1:1.25 Ratio, 22 mg Naproxen”), corresponding to 2.840 mg/mL of unbound naproxen sodium in solution (FIG. 10—“1:1.25 Ratio, 22 mg Naproxen”), and had a binding pH of 8.1 (FIG. 11—“1:1.25 Ratio, 22 mg Naproxen”).

At a 1:1.5 ratio of naproxen sodium to cholestyramine resin, 92.1% of naproxen sodium was bound following the reaction period (FIG. 9—“1:1.5 Ratio, 22 mg Naproxen”), corresponding to 1.740 mg/mL of unbound naproxen sodium in solution (FIG. 10—“1:1.5 Ratio, 22 mg Naproxen”), and had a binding pH of 8.1 (FIG. 11—“1:1.5 Ratio, 22 mg Naproxen”).

At a 1:1.75 ratio of naproxen sodium to cholestyramine resin, 94.9% of naproxen sodium was bound following the reaction period (FIG. 9—“1:1.75 Ratio, 22 mg Naproxen”), corresponding to 1.120 mg/mL of unbound naproxen sodium in solution (FIG. 10—“1:1.75 Ratio, 22 mg Naproxen”), and had a binding pH of 8.1 (FIG. 11—“1:1.75 Ratio, 22 mg Naproxen”).

At a 1:2 ratio of naproxen sodium to cholestyramine resin, 96.5% of naproxen sodium was bound following the reaction period (FIG. 9—“1:2 Ratio, 22 mg Naproxen”), corresponding to 0.770 mg/mL of unbound naproxen sodium in solution (FIG. 10—“1:2 Ratio, 22 mg Naproxen”), and had a binding pH of 8.1 (FIG. 11—“1:2 Ratio, 22 mg Naproxen”).

For solutions initially containing 4.4 g of naproxen sodium (44 mg/mL), at a 1:1.25 ratio of naproxen sodium to cholestyramine resin, 93.8% of naproxen sodium was bound following the reaction period (FIG. 9—“1:1.25 Ratio, 44 mg Naproxen”), corresponding to 2.710 mg/mL of unbound naproxen sodium in solution (FIG. 10—“1:1.25 Ratio, 44 mg Naproxen”), and had a binding pH of 8.6 (FIG. 11—“1:1.25 Ratio, 44 mg Naproxen”).

At a 1:1.5 ratio of naproxen sodium to cholestyramine resin, 96.3% of naproxen sodium was bound following the reaction period (FIG. 9—“1:1.5 Ratio, 44 mg Naproxen”), corresponding to 1.640 mg/mL of unbound naproxen sodium in solution (FIG. 10—“1:1.5 Ratio, 44 mg Naproxen”), and had a binding pH of 8.7 (FIG. 11—“1:1.5 Ratio, 44 mg Naproxen”).

At a 1:1.75 ratio of naproxen sodium to cholestyramine resin, 97.7% of naproxen sodium was bound following the reaction period (FIG. 9—“1:1.75 Ratio, 44 mg Naproxen”), corresponding to 1.020 mg/mL of unbound naproxen sodium in solution (FIG. 10—“1:1.75 Ratio, 44 mg Naproxen”), and had a binding pH of 8.6 (FIG. 11—“1:1.75 Ratio, 44 mg Naproxen”).

At a 1:2 ratio of naproxen sodium to cholestyramine resin, 98.4% of naproxen sodium was bound following the reaction period (FIG. 9—“1:2 Ratio, 44 mg Naproxen”), corresponding to 0.710 mg/mL of unbound naproxen sodium in solution (FIG. 10—“1:2 Ratio, 44 mg Naproxen”), and had a binding pH of 8.6 (FIG. 11—“1:2 Ratio, 44 mg Naproxen”).

For solutions initially containing 8.8 g of naproxen sodium (88 mg/mL), at a 1:1.25 ratio of naproxen sodium to cholestyramine resin, 97.3% of naproxen sodium was bound following the reaction period (FIG. 9—“1:1.25 Ratio, 88 mg Naproxen”), corresponding to 2.410 mg/mL of unbound naproxen sodium in solution (FIG. 10—“1:1.25 Ratio, 88 mg Naproxen”), and had a binding pH of 9.1 (FIG. 11—“1:1.25 Ratio, 88 mg Naproxen”).

At a 1:1.5 ratio of naproxen sodium to cholestyramine resin, 98.3% of naproxen sodium was bound following the reaction period (FIG. 9-“1:1.5 Ratio, 88 mg Naproxen”), corresponding to 1.470 mg/mL of unbound naproxen sodium in solution (FIG. 10—“1:1.5 Ratio, 88 mg Naproxen”), and had a binding pH of 9.1 (FIG. 11—“1:1.5 Ratio, 88 mg Naproxen”).

At a 1:1.75 ratio of naproxen sodium to cholestyramine resin, 98.9% of naproxen sodium was bound following the reaction period (FIG. 9—“1:1.75 Ratio, 88 mg Naproxen”), corresponding to 0.950 mg/mL of unbound naproxen sodium in solution (FIG. 10—“1:1.75 Ratio, 88 mg Naproxen”), and had a binding pH of 9.2 (FIG. 11—“1:1.75 Ratio, 88 mg Naproxen”).

At a 1:2 ratio of naproxen sodium to cholestyramine resin, 99.2% of naproxen sodium was bound following the reaction period (FIG. 9—“1:2 Ratio, 88 mg Naproxen”), corresponding to 0.680 mg/mL of unbound naproxen sodium in solution (FIG. 10—“1:2 Ratio, 88 mg Naproxen”), and had a binding pH of 9.1 (FIG. 11—“1:2 Ratio, 88 mg Naproxen”).

Example 7 demonstrates that a 1:1.5 ratio of naproxen sodium to cholestyramine resin is sufficient to bind naproxen sodium to eliminate chemesthetic effects, i.e., an unbound naproxen sodium concentration of less than 2 mg/mL. For the naproxen sodium batch concentration of 22 mg/mL, a 1:1.5 ratio of naproxen sodium to cholestyramine would result in ˜1.7-1.8 mg/mL free naproxen sodium, or, ˜8% unbound naproxen. During the batch manufacturing process, the reaction between naproxen sodium and cholestyramine resin was designed to be performed at ˜50% total batch volume, i.e., at ˜44 mg/mL naproxen sodium. Example 7 demonstrates that increasing initial naproxen sodium concentration slightly increases the naproxen sodium binding efficiency. Accordingly, the 1:1.5 ratio of naproxen sodium to cholestyramine resin was used in further experiments for dosage form formulation.

### Example 8

Small batches of liquid dosage forms were prepared with varying degrees of excipients to optimize the viscosity of the resulting dosage forms. The formulations developed are shown below in Table 1.

A base batch was prepared by dissolving 22 g of naproxen sodium in 550 g water. 33 g of cholestyramine resin was added to the naproxen sodium solution. The solution was mixed for 1 hr at room temperature. 300 g of xylitol was added to the solution and dissolved, using heat to maintain the solution temperature at 25° C. The base batch was agitated to ensure the solution was homogenous, and subsequently partitioned into 5-183.75 g samples. To each sample, 30 g of glycerin was added and each sample was mixed until miscible. An amount of xanthan gum was added to each sample and each sample was mixed for at least 30 min to disperse. An amount of magnesium aluminometasilicate was added to each sample and each sample was mixed for at least 30 min to disperse. Water was added to each sample to bring the final weight of each sample to 220 g, and each sample was mixed for 1 hr. The viscosity of each sample was evaluated.

TABLE 1

| Compound | Formulation 1A | Formulation 1B | Formulation 1C | Formulation 1D | Formulation 1E | Unit |
|---|---|---|---|---|---|---|
| Naproxen Sodium | 2.20 | 2.20 | 2.20 | 2.20 | 2.20 | (w/v) |
| Cholestyramine Resin | 3.30 | 3.30 | 3.30 | 3.30 | 3.30 | (w/v) |
| Xylitol | 30.00 | 30.00 | 30.00 | 30.00 | 30.00 | (w/v) |
| Sucralose | 0.40 | 0.40 | 0.40 | 0.40 | 0.40 | (w/v) |
| Glycerin | 15.00 | 15.00 | 15.00 | 15.00 | 15.00 | (w/v) |
| Xanthan Gum | 0.50 | 0.50 | 0.35 | 0.25 | 0.35 | (w/v) |
| Magnesium Aluminometasilicate | 0.25 | 0.20 | 0.20 | 0.15 | — | (w/v) |
| Grape Flavor | 0.30 | 0.30 | 0.30 | 0.30 | 0.30 | (w/v) |
| Water | 57.95 | 57.95 | 57.95 | 57.95 | 57.95 | (w/v) |

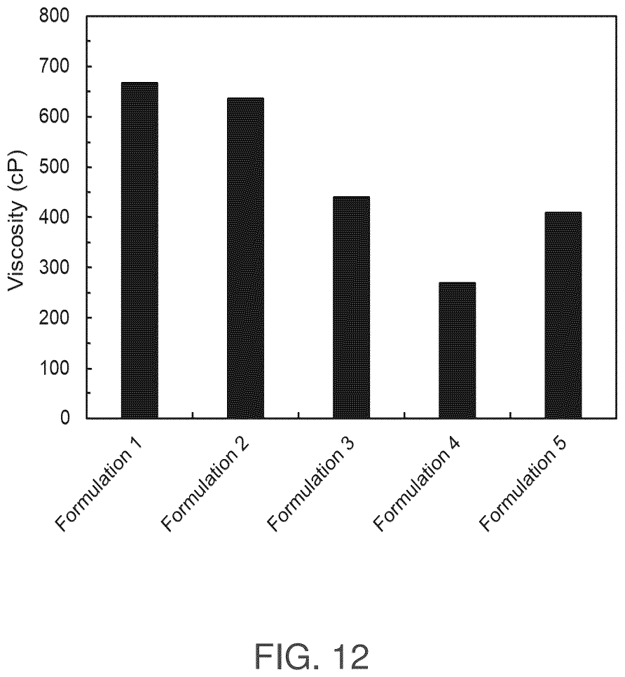

The formulations of Table 1 were evaluated for viscosity and mouth feel. The viscosity values for the formulations of Table 1 are shown in FIG. 12, and were found to be 667.1 cP for Formulation 1A, 636.0 cP for Formulation 1B, 439.2 cP for Formulation 1C, 270.0 cP for Formulation 1D, and 409.2 cP for Formulation 1E.

Among these, Formulation 1C was selected for having the most favorable viscosity and mouth feel.

### Example 9

A 2 L batch of Formulation 1C from Example 8 was prepared by scaling up the method described in Example 8 and preserved for in-house stability testing. The 2 L batch of Formulation 1C from Example 8 is shown below in Table 2.

TABLE 2
2 L Batch

| Compound | Amount (w/v) | Amount (w/w) |
|---|---|---|
| Naproxen Sodium | 2.20 | 2.00 |
| Cholestyramine Resin | 3.30 | 3.00 |
| Xylitol | 30.00 | 27.27 |
| Sucralose | 0.40 | 0.36 |
| Glycerin | 15.00 | 13.64 |
| Xanthan Gum | 0.35 | 0.32 |
| Magnesium Aluminometasilicate | 0.20 | 0.18 |
| Grape Flavor | 0.30 | 0.27 |
| Water | 57.95 | 52.68 |

| | |
|---|---|
| Density | 1.10 g/mL |

In addition, a 20 L scale up reaction was performed to confirm that the binding efficiency of sodium naproxen was preserved at a larger scale. The formulation used in the 20 L scale up reaction is shown below in Table 3.

TABLE 3
20 L Batch

| Compound | Amount (w/v) | Amount (w/w) |
|---|---|---|
| Naproxen Sodium | 2.20 | 2.00 |
| Cholestyramine Resin | 3.30 | 3.00 |
| Xylitol | 30.00 | 27.27 |
| Sucralose | 0.40 | 0.36 |
| Glycerin | 15.00 | 13.64 |
| Xanthan Gum | 0.35 | 0.32 |
| Magnesium Aluminometasilicate | 0.20 | 0.18 |
| Grape Flavor | 0.50 | 0.45 |
| Water | 58.20 | 52.68 |

| | |
|---|---|
| Grittiness Observed | Yes |
| Final Unbound Naproxen | 1.71 mg/mL |
| Concentration | |
| Density | 1.10 g/mL |

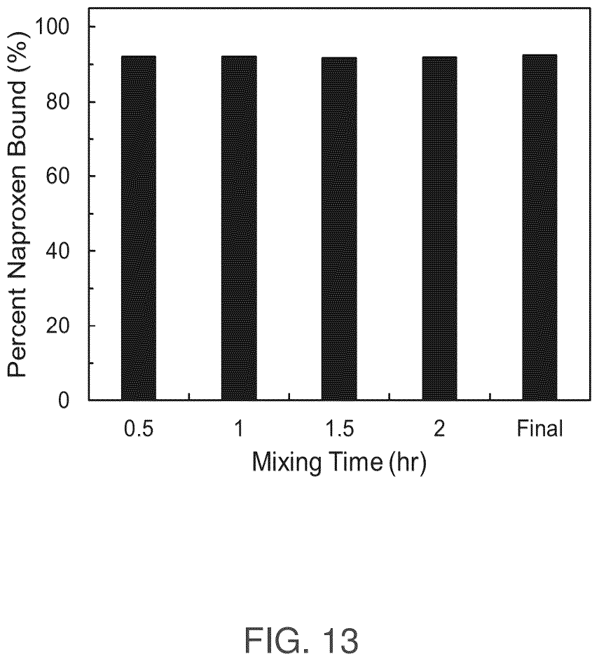

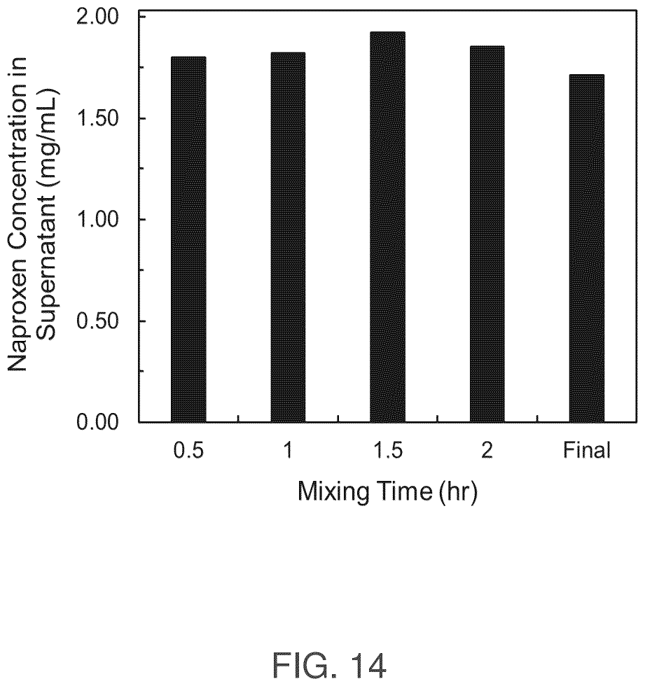

To a 20 L reaction vessel naproxen sodium and cholestyramine resin were dissolved in 11.5 L of water and mixed for 2 hr at room temperature. At selected time points, aliquots were withdrawn to assess unbound naproxen sodium content. After 2 hr, the reaction vessel was set to a controlled temperature of 25° C. Xylitol was then added and the solution was mixed to dissolve. After xylitol dissolved, sucralose was added and the solution was mixed to dissolve. After sucralose dissolved, glycerin was added and the solution was mixed until miscible. After glycerin was dissolved, xanthan gum was added and the solution was mixed to disperse xanthan gum. After xanthan gum was dispersed, magnesium aluminometasilicate was added and the solution was mixed to disperse. After magnesium aluminometasilicate was dissolved, flavoring was added and the solution was mixed to dissolve. After flavoring was dissolved, water was added to bring the mixture to volume and the solution was mixed. The total mixing time was 5.5 hrs (“Final”). The results are shown in FIGS. 13 and 14.

FIG. 13 shows the percentage of naproxen sodium bound by cholestyramine resin in the formulation following a mixing reaction with cholestyramine resin as a function of the duration of the mixing reaction (“Time of Mixing”). FIG. 14 shows the amount of naproxen sodium that remained in the formulation, i.e., unbound, following a mixing reaction with cholestyramine resin as a function of the duration of the mixing reaction (“Time of Mixing”).

After 0.5 hrs of mixing, 92.1% of naproxen sodium was bound (FIG. 13—“0.5”), corresponding to 1.80 mg/mL of unbound naproxen sodium in the formulation (FIG. 14—“0.5”).

After 1 hr of mixing, 92.0% of naproxen sodium was bound (FIG. 13—“1”), corresponding to 1.82 mg/mL of unbound naproxen sodium in the formulation (FIG. 14—“1”).

After 1.5 hrs of mixing, 91.6% of naproxen sodium was bound (FIG. 13—“1.5”), corresponding to 1.92 mg/mL of unbound naproxen sodium in the formulation (FIG. 14—“1.5”).

After 2 hrs of mixing, 91.9% of naproxen sodium was bound (FIG. 13—“2”), corresponding to 1.85 mg/mL of unbound naproxen sodium in the formulation (FIG. 14—“2”).

After 5.5 hrs of mixing, 92.5% of naproxen sodium was bound (FIG. 13—“Final”), corresponding to 1.71 mg/mL of unbound naproxen sodium in the formulation (FIG. 14—“Final”).

### Example 10

Example 9 demonstrated that a 20 L scale formulation could be prepared that did not have chemesthetic effects, i.e., by having an unbound naproxen sodium concentration of less than 2 mg/mL. The formulation of Example 9, however, possessed a level of grittiness that was undesirable for mouth feel. Accordingly, a 20 L batch with less cholestyramine resin was prepared, the formulation of which is shown below in Table 4.

TABLE 4
20 L Batch

| Compound | Amount (w/v) | Amount (w/w) |
|---|---|---|
| Naproxen Sodium | 2.20 | 1.91 |
| Cholestyramine Resin | 2.75 | 2.39 |
| Xylitol | 30.00 | 26.09 |
| Sucralose | 0.25 | 0.22 |
| Glycerin | 15.00 | 13.04 |
| Xanthan Gum | 0.35 | 0.30 |
| Magnesium Aluminometasilicate | 0.20 | 0.17 |
| Grape Flavor | 0.30 | 0.26 |
| Water | 63.95 | 55.61 |

| | |
|---|---|
| Grittiness Observed | No |
| Final Unbound Naproxen | 2.91 mg/mL |
| Concentration | |
| Density | 1.15 g/mL |

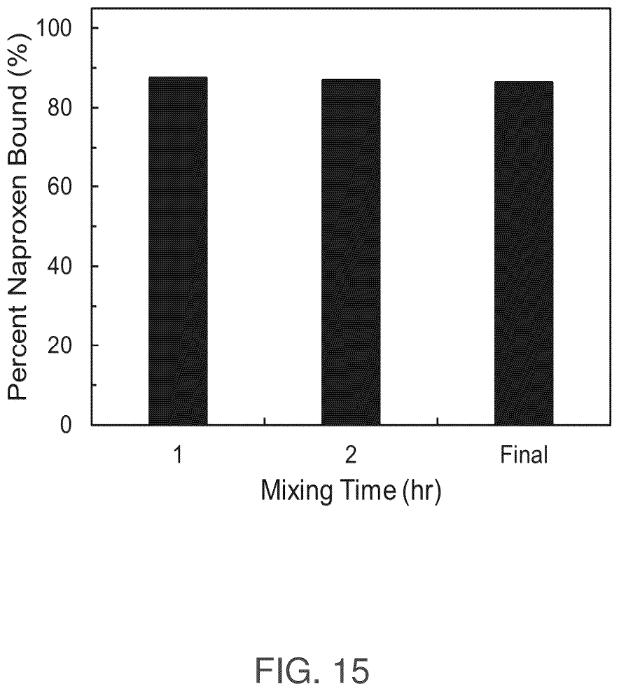

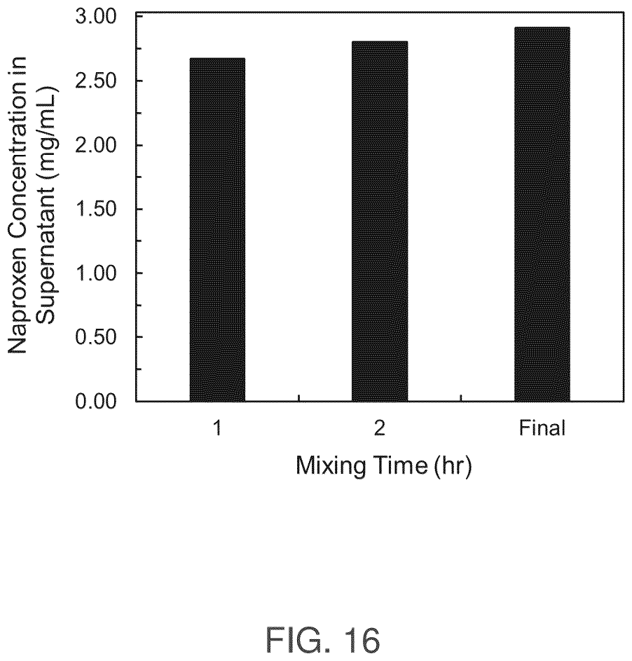

To a 20 L reaction vessel naproxen sodium and cholestyramine resin were dissolved in 11.5 L of water and mixed for 2 hr at room temperature. At selected time points, aliquots were withdrawn to assess unbound naproxen sodium content. After 2 hr, the reaction vessel was set to a controlled temperature of 25° C. Xylitol was then added and the solution was mixed to dissolve. After xylitol dissolved, sucralose was added and the solution was mixed to dissolve. After sucralose dissolved, glycerin was added and the solution was mixed until miscible. After glycerin was dissolved, xanthan gum was added and the solution was mixed to disperse xanthan gum. After xanthan gum was dispersed, magnesium aluminometasilicate was added and the solution was mixed to disperse. After magnesium aluminometasilicate was dissolved, flavoring was added and the solution was mixed to dissolve. After flavoring was dissolved, water was added to bring the mixture to volume and the solution was mixed. The total mixing time was 5.5 hrs (“Final”). The results are shown in FIGS. 15 and 16.

FIG. 15 shows the percentage of naproxen sodium bound by cholestyramine resin in the formulation following a mixing reaction with cholestyramine resin as a function of the duration of the mixing reaction (“Time of Mixing”). FIG. 16 shows the amount of naproxen sodium that remained in the formulation, i.e., unbound, following a mixing reaction with cholestyramine resin as a function of the duration of the mixing reaction (“Time of Mixing”).

After 1 hr of mixing, 87.5% of naproxen sodium was bound (FIG. 15—“1”), corresponding to 2.67 mg/mL of unbound naproxen sodium in the formulation (FIG. 16—“1”).

After 2 hr of mixing, 86.9% of naproxen sodium was bound (FIG. 15—“2”), corresponding to 2.80 mg/mL of unbound naproxen sodium in the formulation (FIG. 16—“2”).

After 5.5 hr of mixing, 86.4% of naproxen sodium was bound (FIG. 15—“Final”), corresponding to 2.91 mg/mL of unbound naproxen sodium in the formulation (FIG. 16—“Final”).

### Example 11

Examples 9-10 collectively demonstrated that (i) using a naproxen sodium to cholestyramine resin at a ratio of 1:1.5 resulted in a formulation with an undesirable mouth feel, i.e., grittiness, and (ii) using a naproxen sodium to cholestyramine resin with a ratio of 1:1.25 resulted in a formulation that may have undesirable chemesthetic effects, i.e., more than 2 mg/mL of unbound naproxen sodium. In an attempt to address these concerns, an additional batch using a naproxen sodium to cholestyramine resin with a ratio of 1:1.5, with an increased concentration of thickener, i.e., xanthan gum, was prepared. This formulation was prepared at a 185 L scale and is shown below in Table 5.

TABLE 5
185 L Batch

| Compound | Amount (w/v) | Amount (w/w) |
|---|---|---|
| Naproxen Sodium | 2.20 | 1.91 |
| Cholestyramine Resin | 3.30 | 2.87 |
| Xylitol | 30.00 | 26.09 |
| Sucralose | 0.40 | 0.35 |
| Glycerin | 15.00 | 13.04 |
| Xanthan Gum | 0.40 | 0.35 |
| Magnesium Aluminometasilicate | 0.20 | 0.17 |
| Grape Flavor | 0.50 | 0.43 |
| Water | 63.00 | 54.79 |

| | |
|---|---|
| Grittiness Observed | No |
| Final Unbound Naproxen | 1.91 mg/mL |
| Concentration | |
| Density | 1.1506 g/mL |
| Viscosity | 395 cP |

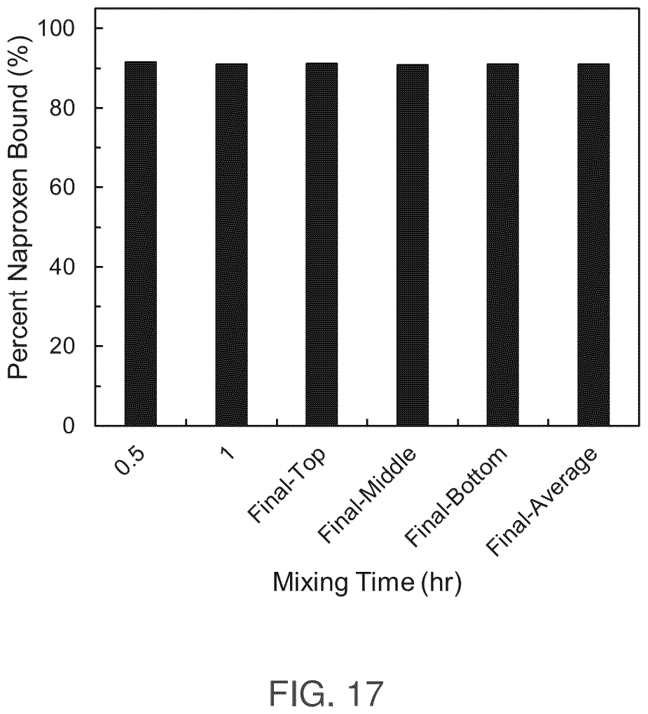

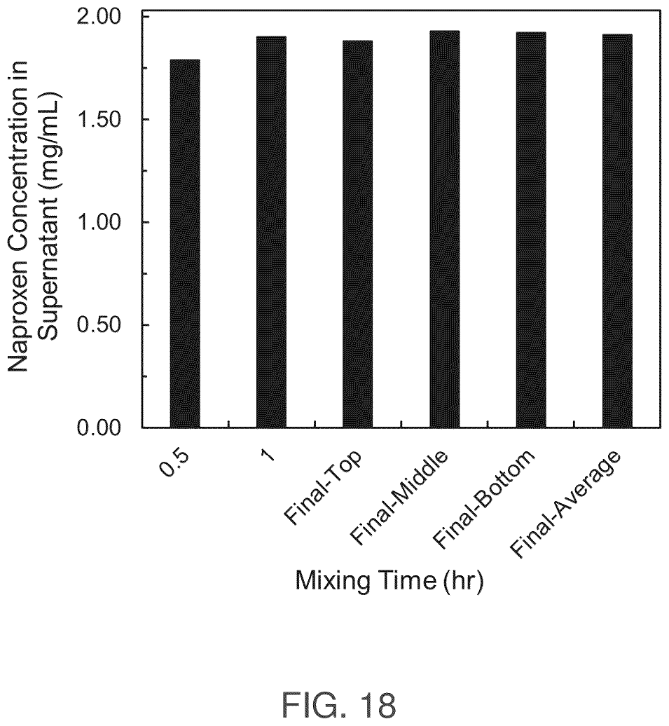

To a 185 L reaction vessel naproxen sodium and cholestyramine resin were dissolved in 105 L of water and mixed for 1 hr at room temperature. At selected time points, aliquots were withdrawn to assess unbound naproxen sodium content. After 1 hr, the reaction vessel was set to a controlled temperature of 25° C. Xylitol was then added and the solution was mixed to dissolve. After xylitol dissolved, sucralose was added and the solution was mixed to dissolve. After sucralose dissolved, glycerin was added and the solution was mixed until miscible. After glycerin was dissolved, xanthan gum was added and the solution was mixed to disperse xanthan gum. After xanthan gum was dispersed, magnesium aluminometasilicate was added and the solution was mixed to disperse. After magnesium aluminometasilicate was dissolved, flavoring was added and the solution was mixed to dissolve. After flavoring was dissolved, water was added to bring the mixture to volume and the solution was mixed. The total mixing time was 8 hrs (“Final”), after which a sample was withdrawn from the top, middle, and bottom of the reactor for analysis. The results are shown in FIGS. 17 and 18.

FIG. 17 shows the percentage of naproxen sodium bound by cholestyramine resin in the formulation following a mixing reaction with cholestyramine resin as a function of the duration of the mixing reaction (“Time of Mixing”). FIG. 18 shows the amount of naproxen sodium that remained in the formulation, i.e., unbound, following a mixing reaction with cholestyramine resin as a function of the duration of the mixing reaction (“Time of Mixing”).

After 0.5 hrs of mixing, 91.6% of naproxen sodium was bound (FIG. 17—“0.5”), corresponding to 1.79 mg/mL of unbound naproxen sodium in the formulation (FIG. 18—“0.5”).

After 1 hr of mixing, 91.1% of naproxen sodium was bound (FIG. 17—“1”), corresponding to 1.91 mg/mL of unbound naproxen sodium in the formulation (FIG. 18—“1”).

After 8 hrs of mixing, 91.2% of naproxen sodium was bound at the top portion of the reactor (FIG. 17—“Final-Top”), corresponding to 1.88 mg/mL of unbound naproxen sodium in the formulation (FIG. 18—“Final-Top”); 90.9% of naproxen sodium was bound at the middle portion of the reactor (FIG. 17—“Final-Middle”), corresponding to 1.93 mg/mL of unbound naproxen sodium in the formulation (FIG. 18—“Final-Middle”); and 91.0% of naproxen sodium was bound at the bottom portion of the reactor (FIG. 17—“Final-Bottom”), corresponding to 1.92 mg/mL of unbound naproxen sodium in the formulation (FIG. 18—“Final-Bottom”). The top, middle, and bottom portions of the reactor were averaged and demonstrated an average of 91.0% of naproxen sodium was bound in the formulation (FIG. 17—“Final-Average”), corresponding to 1.91 mg/mL of unbound naproxen sodium in the formulation (FIG. 18—“Final-Average”).

### Example 12

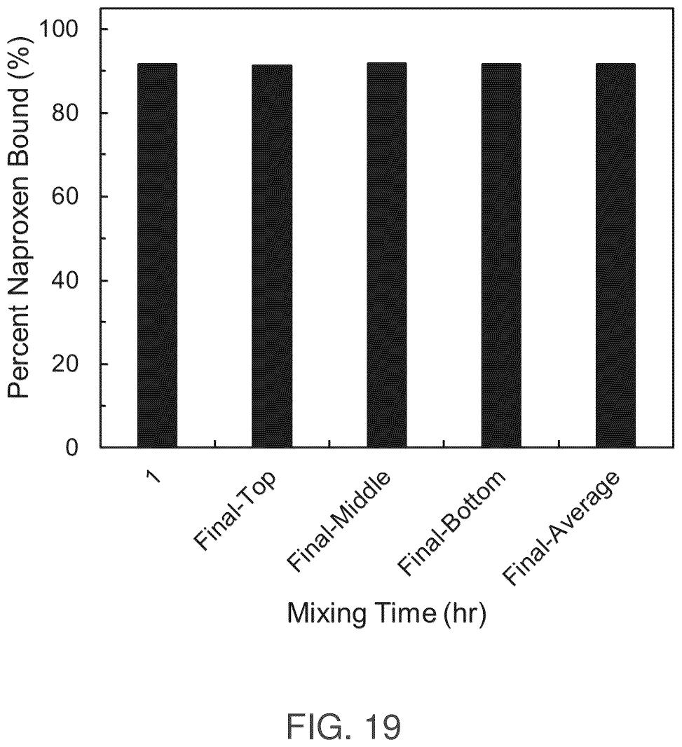

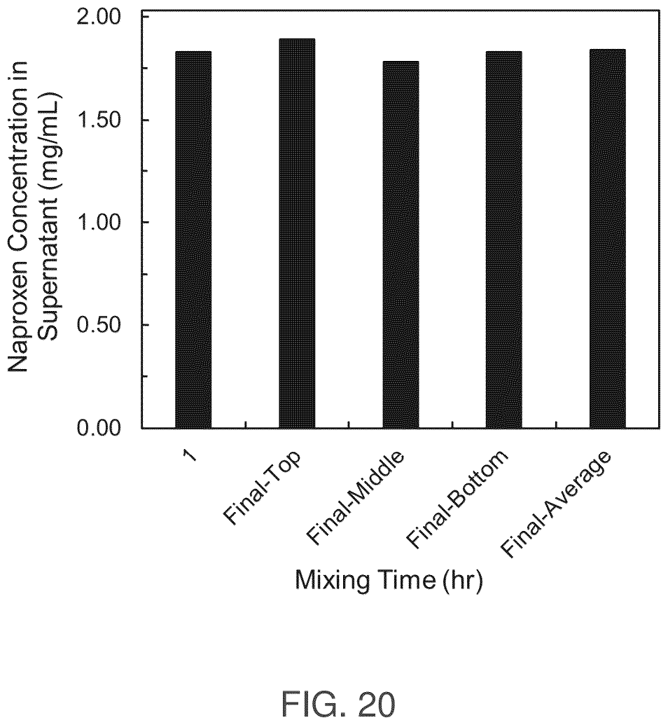

The results from Example 11 demonstrated that a formulation having a desirable mouth feel and lacking chemesthetic effects was obtained. A second 185 L batch having the same composition was prepared via the method described in Example 11, with the only differences being that in Example 12, the initial volume of water used was 100 L and the total mixing time was 8.5 hrs (“Final”). The second 185 L scale formulation is shown below in Table 6. The results are shown in FIGS. 19 and 20.

TABLE 6
185 L Batch

| Compound | Amount (w/v) | Amount (w/w) |
|---|---|---|
| Naproxen Sodium | 2.20 | 1.91 |
| Cholestyramine Resin | 3.30 | 2.87 |
| Xylitol | 30.00 | 26.09 |
| Sucralose | 0.40 | 0.35 |
| Glycerin | 15.00 | 13.04 |
| Xanthan Gum | 0.40 | 0.35 |
| Magnesium Aluminometasilicate | 0.20 | 0.17 |
| Grape Flavor | 0.50 | 0.43 |
| Water | 63.00 | 54.79 |

| | |
|---|---|
| Grittiness Observed | No |
| Final Unbound Naproxen | 1.84 mg/mL |
| Concentration | |
| Density | 1.1492 g/mL |
| Viscosity | 393 cP |

FIG. 19 shows the percentage of naproxen sodium bound by cholestyramine resin in the formulation following a mixing reaction with cholestyramine resin as a function of the duration of the mixing reaction (“Time of Mixing”). FIG. 20 shows the amount of naproxen sodium that remained in the formulation, i.e., unbound, following a mixing reaction with cholestyramine resin as a function of the duration of the mixing reaction (“Time of Mixing”).

After 1 hr of mixing, 91.6% of naproxen sodium was bound (FIG. 19—“1”), corresponding to 1.83 mg/mL of unbound naproxen sodium in the formulation (FIG. 20—“1”).

After 8.5 hrs of mixing, 91.2% of naproxen sodium was bound at the top portion of the reactor (FIG. 18—“Final-Top”), corresponding to 1.89 mg/mL of unbound naproxen sodium in the formulation (FIG. 20—“Final-Top”); 91.9% of naproxen sodium was bound at the middle portion of the reactor (FIG. 19—“Final-Middle”), corresponding to 1.78 mg/mL of unbound naproxen sodium in the formulation (FIG. 20—“Final-Middle”); and 91.7% of naproxen sodium was bound at the bottom portion of the reactor (FIG. 19—“Final-Bottom”), corresponding to 1.83 mg/mL of unbound naproxen sodium in the formulation (FIG. 20—“Final-Bottom”). The top, middle, and bottom portions of the reactor were averaged and demonstrated an average of 91.6% of naproxen sodium was bound in the formulation (FIG. 19—“Final-Average”), corresponding to 1.84 mg/mL of unbound naproxen sodium in the formulation (FIG. 20—“Final-Average”).

In addition, stability testing data for this formulation were obtained. Table 7 below shows the appearance, taste, pH, viscosity, assay, related compounds content, and redispersibility of the formulation.

TABLE 7
185 L Batch-Initial Stability Testing Data

| Test | Criteria | Result |
|---|---|---|
| Appearance | White, Opaque, Uniform | Conforms |
| | Suspension | |
| Taste | Report Results | Sweet grape flavor and smell, no |
| | | throat irritation, no gritiness |
| pH | Report Results | 7.27 |
| Viscosity | Report Results | Top - 393.1 cP |
| | | Middle - 392.4 cP |
| | | Bottom - 393.1 cP |
| Assay | 90.0-110.0% LC | Top - 96.3% |
| | | Middle - 98.4% |
| | | Bottom - 99.0% |
| Related | Report Results | Top - 0.00% |
| Compounds | | Middle - 0.00% |
| | | Bottom - 0.00% |
| Redispers- | Report Results | Top - 98.5% |
| ibility | | Bottom - 94.9% |

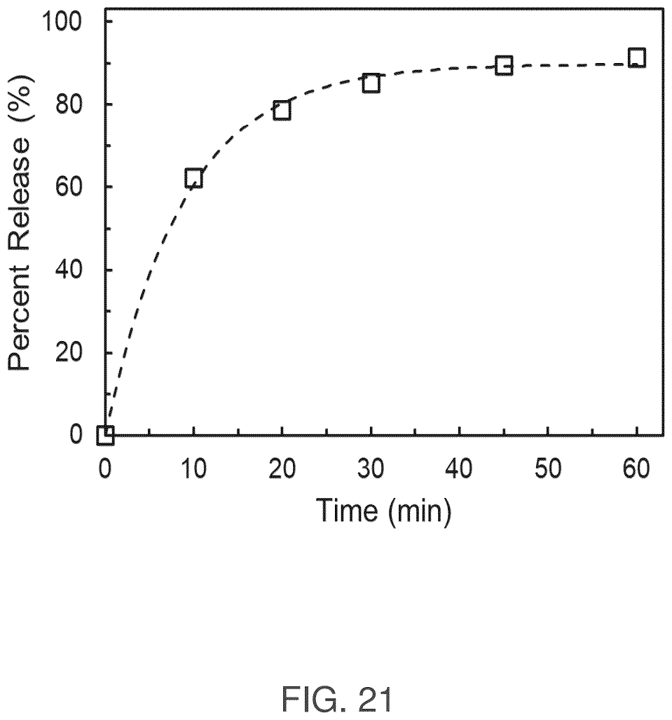

Further, the dissolution properties of the formulation were characterized by dissolution using USP Apparatus 2 (paddles) at 50 RPM in a volume of 900 mL comprising 0.04% aqueous SDS at 37° C. The results of the dissolution study are shown in FIG. 21. In FIG. 21, the formulation released 62.2% of bound naproxen sodium after 10 minutes, 78.6% of bound naproxen sodium after 20 minutes, about 85.2% of bound naproxen sodium after 30 minutes, about 89.5% of bound naproxen sodium after 45 minutes, and 91.4% of bound naproxen sodium after 60 minutes.

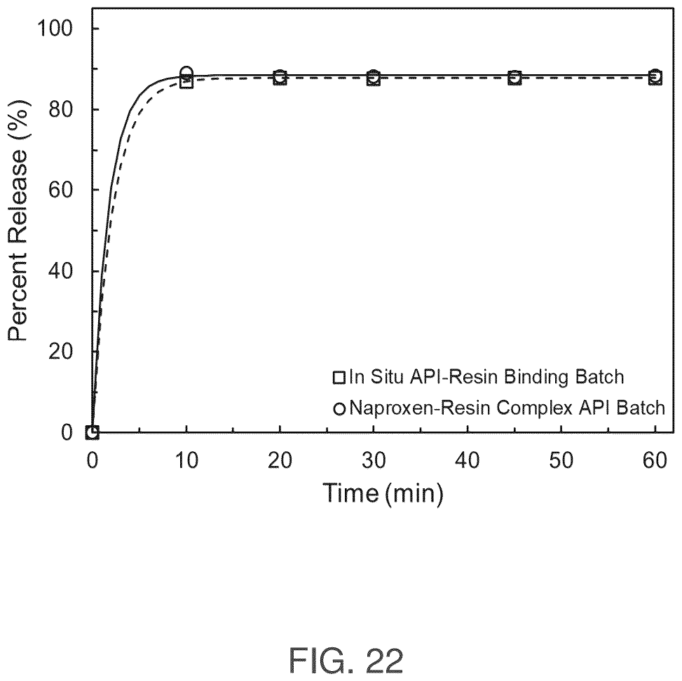

In addition, the dissolution properties of the formulation (“In Situ API-Resin Binding Batch”) were characterized by a dissolution test using USP Apparatus 2 (paddles) at 50 RPM in a volume of 900 mL 1.0 M NaCl solution and compared to the Naproxen-Resin Complex API Batch of Example 1. The results of the comparative dissolution test are shown in FIG. 22. Unexpectedly, the In Situ API-Resin Binding Batch of Example 12 displayed nearly identical dissolution characteristics to the Naproxen-Resin Complex API Batch of Example 1, indicating that neither binding to cholestyramine resin nor incorporation into an aqueous suspension matrix had an impact on dissolution of naproxen sodium.

Lastly, the formulation was evaluated with antimicrobial effectiveness testing (AET) in accordance with USP <51>, Burkholderia cepacia complex (BCC) testing in accordance with USP <60>, total aerobic microbial count (TAMC) testing and total yeast and mold count testing in accordance with USP <61>, and microbiological examination in accordance with USP <62>. The formulation had passing results for the BCC testing, TAMC testing, total yeast and mold count testing, and microbiological examination. At the 14 day interval, the formulation passed the AET, but failed between the 28 day AET test interval.

### Example 13

The results from Example 12 demonstrated that a large scale batch formulation having a desirable mouth feel and lacking chemesthetic effects can be produced. In Example 12, xylitol was employed for its dual purpose as a sweetener and a preservative. The formulation of Example 12, however, lacked sufficient preservative ability in view of the AET. Accordingly, formulations having similar compositions to Example 12 (Table 6), and included one or more additional preservatives, were prepared and evaluated. The results of these experiments are presented in Examples 13-22. A 1 L scale batch having the formulation shown below in Table 8 was prepared.

TABLE 8
1 L Batch

| Compound | Amount (w/v) | Amount (w/w) |
|---|---|---|
| Naproxen Sodium | 2.20 | 1.91 |
| Cholestyramine Resin | 3.30 | 2.87 |
| Xylitol | 30.00 | 26.09 |
| Potassium Sorbate | 0.20 | 0.17 |
| Sucralose | 0.40 | 0.35 |
| Glycerin | 15.00 | 13.04 |
| Xanthan Gum | 0.40 | 0.35 |
| Magnesium Aluminometasilicate | 0.20 | 0.17 |
| Grape Flavor | 0.20 | 0.17 |
| Water | 63.10 | 54.87 |

| | |
|---|---|
| Grittiness Observed | No |
| Final Unbound Naproxen | 2.15 mg/mL |
| Sodium Concentration | |
| Final Unbound Potassium | 1.41 mg/mL |
| Sorbate Concentration | |
| Density | 1.1458 g/mL |

Naproxen sodium was dissolved in 500 mL water. Cholestyramine resin was added and the solution was mixed for 1 hr. Xylitol was added and the solution was mixed for at least 1 hr to dissolve, while maintaining temperature at 25° C. Potassium sorbate was added and the solution was mixed for at least 30 min to dissolve. Sucralose was added and the solution was mixed for at least 30 min to dissolve. Glycerin was added and the solution was mixed for at least 5 min until miscible. Xanthan gum was added and the solution was mixed for at least 30 min to disperse xanthan gum. Magnesium aluminometasilicate was added and the solution was mixed for at least 30 min to disperse. Grape flavor was added and the solution was mixed for at least 15 min to dissolve. Water was added up to final batch weight, and the batch was mixed for at least 1 hr. The total batch mixing time was approximately 5.5 hrs, after which a sample was withdrawn from the batch for analysis of final unbound naproxen sodium content and final unbound potassium sorbate content.

After approximately 5.5 hrs of mixing, there was 2.15 mg/mL of unbound naproxen sodium and 1.41 mg/mL of unbound potassium sorbate in the formulation. The formulation possessed a desirable level of grittiness and a desirable taste profile, and consistent with the unbound naproxen sodium concentration being more than 2 mg/mL, the formulation had minor undesirable chemesthetic effects.

### Example 14

Because the formulation of Example 13 had minor undesirable chemesthetic effects, i.e., an unbound naproxen sodium above 2 mg/mL, the formulation of Example 13 was adjusted. A 1 L scale batch having the formulation shown below in Table 9 was prepared.

TABLE 9
1 L Batch

| Compound | Amount (w/v) | Amount (w/w) |
|---|---|---|
| Naproxen Sodium | 2.20 | 1.91 |
| Cholestyramine Resin | 3.30 | 2.87 |
| Xylitol | 15.00 | 13.04 |
| Potassium Sorbate | 0.20 | 0.17 |
| Sucralose | 0.40 | 0.35 |
| Glycerin | 15.00 | 13.04 |
| Xanthan Gum | 0.40 | 0.35 |
| Magnesium Aluminometasilicate | 0.20 | 0.17 |
| Grape Flavor | 0.20 | 0.17 |
| Water | 78.10 | 67.91 |

| | |
|---|---|
| Grittiness Observed | No |
| Final Unbound Naproxen | 2.07 mg/mL |
| Sodium Concentration | |
| Final Unbound Potassium | 1.39 mg/mL |
| Sorbate Concentration | |
| Density | 1.0966 g/mL |

Naproxen sodium was dissolved in 500 mL water. Cholestyramine resin was added and the solution was mixed for 1 hr. Xylitol was added and the solution was mixed for at least 1 hr to dissolve, while maintaining temperature at 25° C. Potassium sorbate was added and the solution was mixed for at least 30 min to dissolve. Sucralose was added and the solution was mixed for at least 30 min to dissolve. Glycerin was added and the solution was mixed for at least 5 min until miscible. Xanthan gum was added and the solution was mixed for at least 30 min to disperse xanthan gum. Magnesium aluminometasilicate was added and the solution was mixed for at least 30 min to disperse. Grape flavor was added and the solution was mixed for at least 15 min to dissolve. Water was added up to final batch weight, and the batch was mixed for at least 1 hr. The total batch mixing time was approximately 5.5 hrs, after which a sample was withdrawn from the batch for analysis of final unbound naproxen sodium content and final unbound potassium sorbate content.

After approximately 5.5 hrs of mixing, there was 2.07 mg/mL of unbound naproxen sodium and 1.39 mg/mL of unbound potassium sorbate in the formulation. The formulation possessed a desirable level of grittiness and a desirable taste profile, and consistent with the unbound naproxen sodium concentration being more than 2 mg/mL, the formulation had minor undesirable chemesthetic effects.

### Example 15

Because the formulation of Example 14 had minor undesirable chemesthetic effects, i.e., an unbound naproxen sodium above 2 mg/mL, the formulation of Example 14 was adjusted. A 1 L scale batch having the formulation shown below in Table 10 was prepared.

TABLE 10
1 L Batch

| Compound | Amount (w/v) | Amount (w/w) |
|---|---|---|
| Naproxen Sodium | 2.20 | 2.00 |
| Cholestyramine Resin | 3.30 | 3.00 |
| Potassium Sorbate | 0.20 | 0.18 |
| Sucralose | 0.40 | 0.36 |
| Glycerin | 15.00 | 13.64 |
| Xanthan Gum | 0.40 | 0.36 |
| Magnesium Aluminometasilicate | 0.20 | 0.18 |
| Grape Flavor | 0.20 | 0.18 |
| Water | 88.10 | 80.09 |

| | |
|---|---|
| Grittiness Observed | No |
| Final Unbound Naproxen | 2.03 mg/mL |
| Sodium Concentration | |
| Final Unbound Potassium | 1.36 mg/mL |
| Sorbate Concentration | |
| Density | 1.0497 g/mL |

Naproxen sodium was dissolved in 500 mL water. Cholestyramine resin was added and the solution was mixed for 1 hr. Potassium sorbate was added and the solution was mixed for at least 30 min to dissolve. Sucralose was added and the solution was mixed for at least 30 min to dissolve. Glycerin was added and the solution was mixed for at least 5 min until miscible. Xanthan gum was added and the solution was mixed for at least 30 mi to disperse xanthan gum. Magnesium aluminometasilicate was added and the solution was mixed for at least 30 min to disperse. Grape flavor was added and the solution was mixed for at least 15 min to dissolve. Water was added up to final batch weight, and the batch was mixed for at least 1 hr. The total batch mixing time was approximately 4.5 hrs, after which a sample was withdrawn from the batch for analysis of final unbound naproxen sodium content and final unbound potassium sorbate content.

After approximately 4.5 hrs of mixing, there was 2.03 mg/mL of unbound naproxen sodium and 1.36 mg/mL of unbound potassium sorbate in the formulation. The formulation possessed a desirable level of grittiness and a desirable taste profile, and consistent with the unbound naproxen sodium concentration being more than 2 mg/mL, the formulation had minor undesirable chemesthetic effects.

### Example 16

Because the formulation of Example 15 had minor undesirable chemesthetic effects, i.e., an unbound naproxen sodium above 2 mg/mL, the formulation of Example 15 was adjusted. A 1 L scale batch having the formulation shown below in Table 11 was prepared.

TABLE 11
1 L Batch

| Compound | Amount (w/v) | Amount (w/w) |
|---|---|---|
| Naproxen Sodium | 2.20 | 2.10 |
| Cholestyramine Resin | 3.30 | 3.14 |
| Sodium Benzoate | 0.38 | 0.36 |
| Sucralose | 0.40 | 0.38 |
| Glycerin | 15.00 | 14.29 |
| Xanthan Gum | 0.40 | 0.38 |
| Magnesium Aluminometasilicate | 0.20 | 0.19 |
| Grape Flavor | 0.20 | 0.19 |
| Water | 82.93 | 78.98 |

| | |
|---|---|
| Grittiness Observed | No |
| Final Unbound Naproxen | 2.53 mg/mL |
| Sodium Concentration | |
| Final Unbound Sodium | 2.33 mg/mL |
| Benzoate Concentration | |
| Density | 1.0526 g/mL |

Naproxen sodium was dissolved in 500 mL water. Cholestyramine resin was added and the solution was mixed for 1 hr. Sodium benzoate was added and the solution was mixed for at least 1 hr to dissolve, while maintaining temperature at 25° C. Sucralose was added and the solution was mixed for at least 30 min to dissolve. Glycerin was added and the solution was mixed for at least 5 min until miscible. Xanthan gum was added and the solution was mixed for at least 30 min to disperse xanthan gum. Magnesium aluminometasilicate was added and the solution was mixed for at least 30 min to disperse. Grape flavor was added and the solution was mixed for at least 15 min to dissolve. Water was added up to final batch weight, and the batch was mixed for at least 1 hr. The total batch mixing time was approximately 5 hrs, after which a sample was withdrawn from the batch for analysis of final unbound naproxen sodium content and final unbound sodium benzoate content.

After approximately 5 hrs of mixing, there was 2.53 mg/mL of unbound naproxen sodium and 2.33 mg/mL of unbound sodium benzoate in the formulation. The formulation possessed a desirable level of grittiness and a desirable taste profile, and consistent with the unbound naproxen sodium concentration being more than 2 mg/mL, the formulation had minor undesirable chemesthetic effects.

### Example 17

Because the formulation of Example 16 had minor undesirable chemesthetic effects, i.e., an unbound naproxen sodium above 2 mg/mL, the formulation of Example 16 was adjusted. A 1 L scale batch having the formulation shown below in Table 12 was prepared.

TABLE 12
1 L Batch

| Compound | Amount (w/v) | Amount (w/w) |
|---|---|---|
| Naproxen Sodium | 2.20 | 2.10 |
| Cholestyramine Resin | 3.30 | 3.14 |
| Sodium Benzoate | 0.38 | 0.36 |
| Edetate Sodium | 0.20 | 0.19 |
| Sucralose | 0.40 | 0.38 |
| Glycerin | 15.00 | 14.29 |
| Xanthan Gum | 0.40 | 0.38 |
| Magnesium Aluminometasilicate | 0.20 | 0.19 |
| Grape Flavor | 0.20 | 0.19 |
| Water | 82.73 | 78.79 |

| | |
|---|---|
| Grittiness Observed | No |
| Final Unbound Naproxen | 3.06 mg/mL |
| Sodium Concentration | |
| Final Unbound Sodium | 2.55 mg/mL |
| Benzoate Concentration | |
| Density | 1.0521 g/mL |

Naproxen sodium was dissolved in 500 mL water. Cholestyramine resin was added and the solution was mixed for 1 hr. Edetate sodium was added and the solution was mixed for at least 1 hr to dissolve, while maintaining temperature at 25° C. Sodium benzoate was added and the solution was mixed for at least 1 hr to dissolve. Sucralose was added and the solution was mixed for at least 30 min to dissolve. Glycerin was added and the solution was mixed for at least 5 min until miscible. Xanthan gum was added and the solution was mixed for at least 30 min to disperse xanthan gum. Magnesium aluminometasilicate was added and the solution was mixed for at least 30 min to disperse. Grape flavor was added and the solution was mixed for at least 15 min to dissolve. Water was added up to final batch weight, and the batch was mixed for at least 1 hr. The total batch mixing time was approximately 6 hrs, after which a sample was withdrawn from the batch for analysis of final unbound naproxen sodium content and final unbound sodium benzoate content.

After approximately 6 hrs of mixing, there was 3.06 mg/mL of unbound naproxen sodium and 2.55 mg/mL of unbound sodium benzoate in the formulation. The formulation possessed a desirable level of grittiness, but resulted in undesirable chemesthetic effects, consistent with the unbound naproxen sodium being more than 2 mg/mL and the addition of edetate sodium made the formulation unpalatable.

### Example 18

Because the formulation of Example 17 had an unpalatable taste profile and undesirable chemesthetic effects, i.e., an unbound naproxen sodium above 2 mg/mL, the formulation of Example 17 was adjusted.

Naproxen sodium was dissolved in 500 mL water. Cholestyramine resin was added and the solution was mixed for 1 hr. Xylitol was added and the solution was mixed for at least 1 hr to dissolve, while maintaining temperature at 25° C. Sucralose was added and the solution was mixed for at least 30 min to dissolve. Glycerin was added and the solution was mixed for at least 5 min until miscible. Xanthan gum was added and the solution was mixed for at least 30 min to disperse xanthan gum. Magnesium aluminometasilicate was added and the solution was mixed for at least 30 min to disperse. Polysorbate 80 was added and the solution was mixed for at least 30 min to dissolve. Grape flavor was added and the solution was mixed for at least 15 min to dissolve. Water was added up to final batch weight. The batch was split into two 500 mL aliquots, A and B.

To aliquot A, water was added to the final batch weight, and the batch was mixed for at least 1 hr. The total batch mixing time was approximately 5.5 hrs, after which a sample was withdrawn from the batch for analysis of final unbound naproxen sodium content. The final batch formulation is shown below in Table 13.

TABLE 13
1 L Batch

| Compound | Amount (w/v) | Amount (w/w) |
|---|---|---|
| Naproxen Sodium | 2.20 | 2.00 |
| Cholestyramine Resin | 3.30 | 3.00 |
| Xylitol | 10.00 | 9.09 |
| Polysorbate 80 | 0.25 | 0.23 |
| Sucralose | 0.40 | 0.36 |
| Glycerin | 15.00 | 13.64 |
| Xanthan Gum | 0.40 | 0.36 |
| Magnesium Aluminometasilicate | 0.20 | 0.18 |
| Grape Flavor | 0.20 | 0.18 |
| Water | 78.06 | 70.96 |

| | |
|---|---|
| Grittiness Observed | No |
| Final Unbound Naproxen | 1.88 mg/mL |
| Sodium Concentration | |
| Density | 1.0757 g/mL |
| Viscosity | 314.4 cP |

After approximately 5.5 hrs of mixing, there was 1.88 mg/mL of unbound naproxen sodium in the formulation. The formulation possessed a desirable level of grittiness, lacked undesirable chemesthetic effects, consistent with the unbound naproxen sodium being below 2 mg/mL, and had a desirable taste profile.

Further, the formulation was evaluated with antimicrobial effectiveness testing (AET) in accordance with USP <51>. The formulation failed AET at the 14 day interval.

To aliquot B, additional xylitol was added and the solution was mixed for at least 1 hr to dissolve, while maintaining temperature at 25° C. Additional polysorbate 80 was added and the solution was mixed for at least 30 min to dissolve. Water added to final batch weight, and the batch was mixed for 1 hr. The total batch mixing time was approximately 7 hrs, after which a sample was withdrawn from the batch for analysis of final unbound naproxen sodium content. The final batch formulation is shown below in Table 14.

TABLE 14
1 L Batch

| Compound | Amount (w/v) | Amount (w/w) |
|---|---|---|
| Naproxen Sodium | 2.20 | 2.00 |
| Cholestyramine Resin | 3.30 | 3.00 |
| Xylitol | 20.00 | 18.18 |
| Polysorbate 80 | 0.50 | 0.45 |
| Sucralose | 0.40 | 0.36 |
| Glycerin | 15.00 | 13.64 |
| Xanthan Gum | 0.40 | 0.36 |
| Magnesium Aluminometasilicate | 0.20 | 0.18 |
| Grape Flavor | 0.20 | 0.18 |
| Water | 67.82 | 61.65 |

| | |
|---|---|
| Grittiness Observed | No |
| Final Unbound Naproxen | 1.88 mg/mL |
| Sodium Concentration | |
| Density | 1.0791 g/mL |
| Viscosity | 361.2 cP |

After approximately 7 hrs of mixing, there was 1.88 mg/mL of unbound naproxen sodium in the formulation. The formulation possessed a desirable level of grittiness and lacked undesirable chemesthetic effects, consistent with the unbound naproxen sodium being below 2 mg/mL. The increased polysorbate 80 concentration, however, resulted in an unpalatable taste profile.

Further, the formulation was evaluated with antimicrobial effectiveness testing (AET) in accordance with USP <51>. The formulation passed AET at both the 14 day and 28 day intervals.

### Example 19

The results from Example 18 demonstrated that a formulation having a desirable mouth feel and lacking chemesthetic effects that can be produced. One of the formulations of Example 19 also possessed sufficient preservative ability in view of the AET, but had an undesirable taste profile. Accordingly, further experimentation was undertaken to assess whether removing xylitol from—, increasing xanthan gum content of—, and increasing the sucralose content of—the formulation of Example 18 would maintain/improve preservative ability and/or ameliorate an unpleasant taste profile.

Naproxen sodium was dissolved in 500 mL water. Cholestyramine resin was added and the solution was mixed for 1 hr. Sucralose was added and the solution was mixed for at least 30 min to dissolve, while maintaining temperature at 25° C. Glycerin was added and the solution was mixed at least 5 min until miscible. Xanthan gum was added and the solution was mixed for at least 30 min to disperse xanthan gum. Magnesium aluminometasilicate was added and the solution was mixed for at least 30 min to disperse. Polysorbate 80 was added and the solution was mixed for at least 30 min to dissolve. Grape flavor was added and the solution was mixed for at least 15 min to dissolve. Water was added up to final batch weight. The batch was split into two 500 mL aliquots, A and B.

To aliquot A, water was added to the final batch weight, and the batch was mixed for at least 1 hr. The total batch mixing time was approximately 4.5 hrs, after which a sample was withdrawn from the batch for analysis of final unbound naproxen sodium content. The final batch formulation is shown below in Table 15.

TABLE 15
0.5 L Batch

| Compound | Amount (w/v) | Amount (w/w) |
|---|---|---|
| Naproxen Sodium | 2.20 | 2.10 |
| Cholestyramine Resin | 3.30 | 3.14 |
| Polysorbate 80 | 0.25 | 0.24 |
| Sucralose | 0.60 | 0.57 |
| Glycerin | 15.00 | 14.29 |
| Xanthan Gum | 0.50 | 0.48 |
| Magnesium Aluminometasilicate | 0.20 | 0.19 |
| Grape Flavor | 0.20 | 0.19 |
| Water | 82.74 | 78.80 |

| | |
|---|---|
| Grittiness Observed | No |
| Final Unbound Naproxen | 1.88 mg/mL |
| Sodium Concentration | |
| Density | 1.0345 g/mL |
| Viscosity | 428.4 cP |

After approximately 4.5 hrs of mixing, there was 1.88 mg/mL of unbound naproxen sodium in the formulation. The formulation possessed a desirable level of grittiness and taste profile, and lacked undesirable chemesthetic effects, consistent with the unbound naproxen sodium being below 2 mg/mL.

Further, the formulation was evaluated with antimicrobial effectiveness testing (AET) in accordance with USP <51>. The formulation passed AET at both the 14 day and 28 day intervals.

To aliquot B, additional polysorbate 80 was added and the solution was mixed for at least 30 min to dissolve. Water added to final batch weight, and the batch was mixed for 1 hr. The total batch mixing time was approximately 6 hrs, after which a sample was withdrawn from the batch for analysis of final unbound naproxen sodium content. The final batch formulation is shown below in Table 16.

TABLE 16
0.5 L Batch

| Compound | Amount (w/v) | Amount (w/w) |
|---|---|---|
| Naproxen Sodium | 2.20 | 2.10 |
| Cholestyramine Resin | 3.30 | 3.14 |
| Polysorbate 80 | 0.50 | 0.48 |
| Sucralose | 0.60 | 0.57 |
| Glycerin | 15.00 | 14.29 |
| Xanthan Gum | 0.50 | 0.48 |
| Magnesium Aluminometasilicate | 0.20 | 0.19 |
| Grape Flavor | 0.20 | 0.19 |
| Water | 82.49 | 78.56 |

| | |
|---|---|
| Grittiness Observed | No |
| Final Unbound Naproxen | 1.88 mg/mL |
| Sodium Concentration | |
| Density | 1.0222 g/mL |
| Viscosity | 438.0 cP |

After approximately 6 hrs of mixing, there was 1.88 mg/mL of unbound naproxen sodium in the formulation. The formulation possessed a desirable level of grittiness and taste profile, and lacked undesirable chemesthetic effects, consistent with the unbound naproxen sodium being below 2 mg/mL.

Further, the formulation was evaluated with antimicrobial effectiveness testing (AET) in accordance with USP <51>. The formulation passed AET at both the 14 day and 28 day intervals.

### Example 20

The results from Example 19 demonstrated a formulation having a desirable mouth feel and taste profile, and lacking chemesthetic effects that can be produced. Both of the formulations of Example 19 also passed AET. Further experimentation was undertaken to assess whether formulations including potassium sorbate could have the same desirable features as the formulation from Example 19.

Naproxen sodium was dissolved in 1000 mL water. Cholestyramine resin was added and the solution was mixed for 1 hr. Sucralose was added and the solution was mixed for at least 30 min to dissolve, while maintaining temperature at 25° C. Glycerin was added and the solution was mixed for at least 5 min until miscible. Xanthan gum was added and the solution was mixed for at least 30 min to disperse xanthan gum. Magnesium aluminometasilicate was added and the solution was mixed for at least 30 min to disperse. Grape flavor was added and the solution was mixed for at least 15 min to dissolve. Water was added up to final batch weight. The batch was split into 200 mL aliquots, E and F.

To aliquot E, potassium sorbate and polysorbate 80 were added and the solution was mixed for at least 1 hr to dissolve. Water was added to the final batch weight, and the batch was mixed for at least 1 hr. The total batch mixing time was approximately 5 hrs, after which a sample was withdrawn from the batch for analysis of final unbound naproxen sodium content and unbound potassium sorbate content. The final batch formulation is shown below in Table 17.

TABLE 17
0.2 L Batch

| Compound | Amount (w/v) | Amount (w/w) |
|---|---|---|
| Naproxen Sodium | 2.20 | 2.10 |
| Cholestyramine Resin | 3.30 | 3.14 |
| Polysorbate 80 | 0.50 | 0.48 |
| Potassium Sorbate | 0.20 | 0.19 |
| Sucralose | 0.60 | 0.57 |
| Glycerin | 15.00 | 14.29 |
| Xanthan Gum | 0.50 | 0.48 |
| Magnesium Aluminometasilicate | 0.20 | 0.19 |
| Grape Flavor | 0.20 | 0.19 |
| Water | 82.29 | 78.37 |

| | |
|---|---|
| Grittiness Observed | No |
| Final Unbound Naproxen | 2.12 mg/mL |
| Sodium Concentration | |
| Final Unbound Potassium | 1.34 mg/mL |
| Sorbate Concentration | |
| Density | 1.0524 g/mL |

After approximately 5 hrs of mixing, there was 2.12 mg/mL of unbound naproxen sodium and 1.34 mg/mL of unbound potassium sorbate in the formulation. The formulation possessed a desirable level of grittiness and a desirable taste profile, and consistent with the unbound naproxen sodium concentration being more than 2 mg/mL, the formulation had minor undesirable chemesthetic effects.

Further, the formulation was evaluated with antimicrobial effectiveness testing (AET) in accordance with USP <51>. The formulation passed AET at both the 14 day and 28 day intervals.

To aliquot F, potassium sorbate and polysorbate 80 were added and the solution was mixed for at least 1 hr to dissolve. Water was added to the final batch weight, and the batch was mixed for at least 1 hr. The total batch mixing time was approximately 5 hrs, after which a sample was withdrawn from the batch for analysis of final unbound naproxen sodium content and unbound potassium sorbate content. The final batch formulation is shown below in Table 18.

TABLE 18
0.2 L Batch

| Compound | Amount (w/v) | Amount (w/w) |
|---|---|---|
| Naproxen Sodium | 2.20 | 2.10 |
| Cholestyramine Resin | 3.30 | 3.14 |
| Polysorbate 80 | 0.50 | 0.48 |
| Potassium Sorbate | 0.30 | 0.29 |
| Sucralose | 0.60 | 0.57 |
| Glycerin | 15.00 | 14.29 |
| Xanthan Gum | 0.50 | 0.48 |
| Magnesium Aluminometasilicate | 0.20 | 0.19 |
| Grape Flavor | 0.20 | 0.19 |
| Water | 82.19 | 78.27 |

| | |
|---|---|
| Grittiness Observed | No |
| Final Unbound Naproxen | 2.30 mg/mL |
| Sodium Concentration | |
| Final Unbound Potassium | 2.13 mg/mL |
| Sorbate Concentration | |
| Density | 1.0524 g/mL |

After approximately 5 hrs of mixing, there was 2.30 mg/mL of unbound naproxen sodium and 2.13 mg/mL of unbound potassium sorbate in the formulation. The formulation possessed a desirable level of grittiness and a desirable taste profile, and consistent with the unbound naproxen sodium concentration being more than 2 mg/mL, the formulation had minor undesirable chemesthetic effects.

Further, the formulation was evaluated with antimicrobial effectiveness testing (AET) in accordance with USP <51>. The formulation passed AET at both the 14 day and 28 day intervals.

### Example 21

Because the formulation of Example 20 had minor undesirable chemesthetic effects, i.e., an unbound naproxen sodium above 2 mg/mL, the formulation of Example 20 was adjusted. A 10 L scale batch having the formulation shown below in Table 19 was prepared.

TABLE 19
10 L Batch

| Compound | Amount (w/v) | Amount (w/w) |
|---|---|---|
| Naproxen Sodium | 2.20 | 2.10 |
| Cholestyramine Resin | 3.30 | 3.14 |
| Polysorbate 80 | 0.25 | 0.24 |
| Sucralose | 0.50 | 0.48 |
| Glycerin | 15.00 | 14.29 |
| Xanthan Gum | 0.50 | 0.48 |
| Magnesium Aluminometasilicate | 0.20 | 0.19 |
| Grape Flavor | 0.70 | 0.67 |
| Water | 82.34 | 78.43 |

| | |
|---|---|
| Grittiness Observed | No |
| Final Unbound Naproxen | 1.90 mg/mL |
| Sodium Concentration | |
| Density | 1.0490 g/mL |

Naproxen sodium was dissolved in 5000 mL water. Cholestyramine resin was added and the solution was mixed for 1 hr. Sucralose was added and the solution was mixed for at least 30 min to dissolve. Glycerin was added and the solution was mixed for at least 5 min until miscible. Xanthan gum was added and the solution was mixed for at least 30 min to disperse xanthan gum. Magnesium aluminometasilicate was added and the solution was mixed for at least 30 min to disperse. Polysorbate 80 was added and the solution was mixed for at least 30 min to dissolve. Grape flavor was added and the solution was mixed for at least 15 min to dissolve. Water was added up to final batch weight, and the batch was mixed for at least 1 hr. The total batch mixing time was approximately 5 hrs, after which a sample was withdrawn from the batch for analysis of final unbound naproxen sodium content.

After approximately 5 hrs of mixing, there was 1.90 mg/mL of unbound naproxen sodium in the formulation. The formulation possessed a desirable level of grittiness and taste profile, and lacked undesirable chemesthetic effects, consistent with the unbound naproxen sodium being below 2 mg/mL.

Further, the formulation was evaluated with TAMC testing and total yeast and mold count testing in accordance with USP <61>. The formulation failed both TAMC testing and total yeast and mold count testing.

### Example 22

Because the formulation of Example 21 had failed the TAMC testing and total yeast and mold count testing, the formulation of Example 21 was adjusted. Four 1 L scale batches (“Formulations 1-4”) were prepared.

A first 1 L scale batch having the formulation shown below in Table 20 was prepared (“Formulation 1”).

TABLE 20
1 L Batch

| Compound | Amount (w/v) | Amount (w/w) |
|---|---|---|
| Naproxen Sodium | 2.20 | 2.10 |
| Cholestyramine Resin | 3.30 | 3.14 |
| Polysorbate 80 | 0.50 | 0.48 |
| Propylene Glycol | 5.00 | 4.76 |
| Sucralose | 0.50 | 0.48 |
| Glycerin | 10.00 | 9.52 |
| Xanthan Gum | 0.50 | 0.48 |
| Magnesium Aluminometasilicate | 0.20 | 0.19 |
| Grape Flavor | 0.70 | 0.67 |
| Water | 82.10 | 78.19 |

| | |
|---|---|
| Grittiness Observed | No |
| Final Unbound Naproxen | 1.94 mg/mL |
| Sodium Concentration | |
| Density | 1.0476 g/mL |

To a side vessel, propylene glycol was added. Glycerin was added and the solution was mixed for at least 15 min until miscible. Xanthan gum was added and the solution was mixed for at least 10 min to disperse xanthan gum. Magnesium aluminometasilicate was added and the solution was mixed for at least 10 min to disperse.

To a main vessel, naproxen sodium was dissolved in 500 mL of water. Cholestyramine resin was added and the solution was mixed for at least 1 hr. Sucralose was added and the solution was mixed for at least 30 min to dissolve. The contents of the side vessel were transferred to the main vessel and the solution was mixed for at least 30 min to dissolve. Polysorbate 80 was added and the solution was mixed for at least 30 min dissolve. Grape flavor was added and the solution was mixed for at least 15 min to dissolve. Water was added up to final batch weight, and the batch was mixed for at least 1 hr. The total batch mixing time was approximately 4.5 hrs, after which a sample was withdrawn from the batch for analysis of final unbound naproxen sodium content.

After approximately 4.5 hrs of mixing, there was 1.94 mg/mL of unbound naproxen sodium in Formulation 1. Formulation 1 possessed a desirable level of grittiness, lacked undesirable chemesthetic effects, consistent with the unbound naproxen sodium being below 2 mg/mL, and had a desirable taste profile.

Further, Formulation 1 was evaluated with TAMC testing and total yeast and mold count testing in accordance with USP <61> and AET in accordance with USP <51>. Formulation 1 passed both TAMC testing and total yeast and mold count testing, but failed AET at the 14 day interval.

A second 1 L scale batch having the formulation shown below in Table 21 was prepared (“Formulation 2”).

TABLE 21
1 L Batch

| Compound | Amount (w/v) | Amount (w/w) |
|---|---|---|
| Naproxen Sodium | 2.20 | 2.10 |
| Cholestyramine Resin | 3.30 | 3.14 |
| Polysorbate 80 | 0.50 | 0.48 |
| Methylparaben | 0.15 | 0.14 |
| Propylparaben | 0.05 | 0.05 |
| Propylene Glycol | 5.00 | 4.76 |
| Sucralose | 0.50 | 0.48 |
| Glycerin | 10.00 | 9.52 |
| Xanthan Gum | 0.50 | 0.48 |
| Magnesium Aluminometasilicate | 0.20 | 0.19 |
| Grape Flavor | 0.70 | 0.67 |
| Water | 81.90 | 78.00 |

| | |
|---|---|
| Grittiness Observed | No |
| Final Unbound Naproxen | 1.93 mg/mL |
| Sodium Concentration | |
| Final Unbound | 1.35 mg/mL |
| Methylparaben Concentration | |
| Final Unbound | 0.38 mg/mL |
| Propylparaben Concentration | |
| Density | 1.0389 g/mL |

To a side vessel, propylene glycol was added. Methylparaben was added and the solution was mixed until dissolved. Propylparaben was added and the solution was mixed until dissolved. Glycerin was added and the solution was mixed for at least 15 min until miscible. Xanthan gum was added and the solution was mixed for at least 10 min to disperse xanthan gum. Magnesium aluminometasilicate was added and the solution was mixed for at least 10 min to disperse.

To a main vessel, naproxen sodium was dissolved in 500 mL of water. Cholestyramine resin was added and the solution was mixed for at least 1 hr. Sucralose was added and the solution was mixed for at least 30 min to dissolve. The contents of the side vessel were transferred to the main vessel and the solution was mixed for at least 30 min to dissolve. Polysorbate 80 was added and the solution was mixed for at least 30 min to dissolve. Grape flavor was added and the solution was mixed for at least 15 min to dissolve. Water was added up to final batch weight, and the batch was mixed for at least 1 hr. The total batch mixing time was approximately 5.5 hrs, after which a sample was withdrawn from the batch for analysis of final unbound naproxen sodium content, final unbound methylparaben content, and final unbound propylparaben content.

After approximately 5.5 hrs of mixing, there was 1.93 mg/mL of unbound naproxen sodium, 1.35 mg/mL of unbound methylparaben, and 0.38 mg/mL of unbound propylparaben in Formulation 2. Formulation 2 possessed a desirable level of grittiness, lacked undesirable chemesthetic effects, consistent with the unbound naproxen sodium being below 2 mg/mL, and had a desirable taste profile.

Further, Formulation 2 was evaluated with TAMC testing and total yeast and mold count testing in accordance with USP <61> and AET in accordance with USP <51>. Formulation 2 passed both TAMC testing and total yeast and mold count testing, as well as AET at both the 14 day and 28 day intervals.

A third 1 L scale batch having the formulation shown below in Table 22 was prepared (“Formulation 3”).

TABLE 22
1 L Batch

| Compound | Amount (w/v) | Amount (w/w) |
|---|---|---|
| Naproxen Sodium | 2.20 | 2.10 |
| Cholestyramine Resin | 3.30 | 3.14 |
| Polysorbate 80 | 0.50 | 0.48 |
| Methylparaben | 0.15 | 0.14 |
| Propylene Glycol | 5.00 | 4.76 |
| Sucralose | 0.50 | 0.48 |
| Glycerin | 10.00 | 9.52 |
| Xanthan Gum | 0.50 | 0.48 |
| Magnesium Aluminometasilicate | 0.20 | 0.19 |
| Grape Flavor | 0.70 | 0.67 |
| Water | 81.95 | 78.05 |

| | |
|---|---|
| Grittiness Observed | No |
| Final Unbound Naproxen | 1.94 mg/mL |
| Sodium Concentration | |
| Final Unbound | 1.34 mg/mL |
| Methylparaben Concentration | |
| Density | 1.0485 g/mL |

To a side vessel, propylene glycol was added. Methylparaben was added and the solution was mixed until dissolved. Glycerin was added and the solution was mixed for at least 15 min until miscible. Xanthan gum was added and the solution was mixed for at least 10 min to disperse to xanthan gum. Magnesium aluminometasilicate was added and the solution was mixed for at least 10 min to disperse.

To a main vessel, naproxen sodium was dissolved in 500 mL of water. Cholestyramine resin was added and the solution was mixed for at least 1 hr. Sucralose was added and the solution was mixed for at least 30 min to dissolve. The contents of the side vessel were transferred to the main vessel and the solution was mixed for at least 30 min to dissolve. Polysorbate 80 was added and the solution was mixed for at least 30 min to dissolve. Grape flavor was added and the solution was mixed for at least 15 min to dissolve. Water was added up to final batch weight, and the batch was mixed for at least 1 hr. The total batch mixing time was approximately 5 hrs, after which a sample was withdrawn from the batch for analysis of final unbound naproxen sodium content, and final unbound methylparaben content.

After approximately 5 hrs of mixing, there was 1.94 mg/mL of unbound naproxen sodium, and 1.34 mg/mL of unbound methylparaben in Formulation 3. Formulation 3 possessed a desirable level of grittiness, lacked undesirable chemesthetic effects, consistent with the unbound naproxen sodium being below 2 mg/mL, and had a desirable taste profile.

Further, Formulation 3 was evaluated with TAMC testing and total yeast and mold count testing in accordance with USP <61> and AET in accordance with USP <51>. Formulation 3 passed both TAMC testing and total yeast and mold count testing, but failed AET at the 14 day interval.

A fourth 1 L scale batch having the formulation shown below in Table 23 was prepared (“Formulation 4”).

TABLE 23
1 L Batch

| Compound | Amount (w/v) | Amount (w/w) |
|---|---|---|
| Naproxen Sodium | 2.20 | 2.10 |
| Cholestyramine Resin | 3.30 | 3.14 |
| Polysorbate 80 | 0.50 | 0.48 |
| Propylparaben | 0.05 | 0.05 |
| Propylene Glycol | 5.00 | 4.76 |
| Sucralose | 0.50 | 0.48 |
| Glycerin | 10.00 | 9.52 |
| Xanthan Gum | 0.50 | 0.48 |
| Magnesium Aluminometasilicate | 0.20 | 0.19 |
| Grape Flavor | 0.70 | 0.67 |
| Water | 82.05 | 78.14 |

| | |
|---|---|
| Grittiness Observed | No |
| Final Unbound Naproxen | 1.90 mg/mL |
| Sodium Concentration | |
| Final Unbound | 0.37 mg/mL |
| Propylparaben Concentration | |
| Density | 1.0339 g/mL |

To a side vessel, propylene glycol was added. Propylparaben was added and the solution was mixed until dissolved. Glycerin was added and the solution was mixed for at least 15 min until miscible. Xanthan gum was added and the solution was mixed for at least 10 min to disperse xanthan gum. Magnesium aluminometasilicate was added and the solution was mixed for at least 10 min to disperse.

To a main vessel, naproxen sodium was dissolved in 500 mL of water. Cholestyramine resin was added and the solution was mixed for at least 1 hr. Sucralose was added and the solution was mixed for at least 30 min to dissolve. The contents of the side vessel were transferred to the main vessel and the solution was mixed for at least 30 min to dissolve. Polysorbate 80 was added and the solution was mixed for at least 30 min to dissolve. Grape flavor was added and the solution was mixed for at least 15 min to dissolve. Water was added up to final batch weight, and the batch was mixed for at least 1 hr. The total batch mixing time was approximately 5 hrs, after which a sample was withdrawn from the batch for analysis of final unbound naproxen sodium content, and final unbound propylparaben content.

After approximately 5 hrs of mixing, there was 1.90 mg/mL of unbound naproxen sodium, and 0.37 mg/mL of unbound propylparaben in Formulation 4. Formulation 4 possessed a desirable level of grittiness, lacked undesirable chemesthetic effects, consistent with the unbound naproxen sodium being below 2 mg/mL, and had a desirable taste profile.

Further, Formulation 4 was evaluated with TAMC testing and total yeast and mold count testing in accordance with USP <61> and AET in accordance with USP <51>. Formulation 4 passed both TAMC testing and total yeast and mold count testing, as well as AET at both the 14 day and 28 day intervals.

### Example 23

The results from Example 22 demonstrated a formulation having a desirable mouth feel and taste profile, lacking chemesthetic effects, and capable of passing microbial testing (USP <61> and USP <51>) can be produced by increasing the concentration of polysorbate 80 and including either: (i) propylparaben; or (ii) a combination of methylparaben and propylparaben. Because of concerns that polysorbate 80 could inhibit the preservative effectiveness of methylparaben and/or propylparaben, two additional 1 L scale batches (“Formulations 5-6”) were prepared.

A first 1 L scale batch having the formulation shown below in Table 24 was prepared (“Formulation 5”).

TABLE 24
1 L Batch

| Compound | Amount (w/v) | Amount (w/w) |
|---|---|---|
| Naproxen Sodium | 2.20 | 2.10 |
| Cholestyramine Resin | 3.30 | 3.14 |
| Methylparaben | 0.15 | 0.14 |
| Propylparaben | 0.05 | 0.05 |
| Propylene Glycol | 5.00 | 4.76 |
| Sucralose | 0.50 | 0.48 |
| Glycerin | 10.00 | 9.52 |
| Xanthan Gum | 0.50 | 0.48 |
| Magnesium Aluminometasilicate | 0.20 | 0.19 |
| Grape Flavor | 0.70 | 0.67 |
| Water | 82.39 | 78.47 |

| | |
|---|---|
| Grittiness Observed | No |
| Final Unbound Naproxen | 1.96 mg/mL |
| Sodium Concentration | |
| Final Unbound | 1.34 mg/mL |
| Methylparaben Concentration | |
| Final Unbound | 0.38 mg/mL |
| Propylparaben Concentration | |
| Density | 1.0446 g/mL |

To a side vessel, propylene glycol was added. Methylparaben was added and the solution was mixed until dissolved. Propylparaben was added and the solution was mixed until dissolved. Glycerin was added and the solution was mixed for at least 15 min until miscible. Xanthan gum was added and the solution was mixed for at least 10 min to disperse xanthan gum. Magnesium aluminometasilicate was added and the solution was mixed for at least 10 min to disperse.

To a main vessel, naproxen sodium was dissolved in 500 mL of water. Cholestyramine resin was added and the solution was mixed for at least 1 hr. Sucralose was added and the solution was mixed for at least 30 min to dissolve. The contents of the side vessel were transferred to the main vessel and the solution was mixed for at least 30 min to dissolve. Grape flavor was added and the solution was mixed for at least 15 min to dissolve. Water was added up to final batch weight, and the batch was mixed for at least 1 hr. The total batch mixing time was approximately 5 hrs, after which a sample was withdrawn from the batch for analysis of final unbound naproxen sodium content, and final unbound propylparaben content.

After approximately 5 hrs of mixing, there was 1.96 mg/mL of unbound naproxen sodium, 1.34 mg/mL of unbound methylparaben, and 0.38 mg/mL of unbound propylparaben in Formulation 5. Formulation 5 possessed a desirable level of grittiness, lacked undesirable chemesthetic effects, consistent with the unbound naproxen sodium being below 2 mg/mL, and had a desirable taste profile.

Further, Formulation 5 was evaluated with TAMC testing and total yeast and mold count testing in accordance with USP <61> and AET in accordance with USP <51>. Formulation 5 passed both TAMC testing and total yeast and mold count testing, as well as AET at both the 14 day and 28 day intervals.

A second 1 L scale batch having the formulation shown below in Table 25 was prepared (“Formulation 6”).

TABLE 25
1 L Batch

| Compound | Amount (w/v) | Amount (w/w) |
|---|---|---|
| Naproxen Sodium | 2.20 | 2.10 |
| Cholestyramine Resin | 3.30 | 3.14 |
| Methylparaben | 0.30 | 0.29 |
| Propylparaben | 0.10 | 0.10 |
| Propylene Glycol | 5.00 | 4.76 |
| Sucralose | 0.50 | 0.48 |
| Glycerin | 10.00 | 9.52 |
| Xanthan Gum | 0.50 | 0.48 |
| Magnesium Aluminometasilicate | 0.20 | 0.19 |
| Grape Flavor | 0.70 | 0.67 |
| Water | 82.18 | 78.27 |

| | |
|---|---|
| Grittiness Observed | No |
| Final Unbound Naproxen | 2.00 mg/mL |
| Sodium Concentration | |
| Final Unbound | 2.75 mg/mL |
| Methylparaben Concentration | |
| Final Unbound | 0.78 mg/mL |
| Propylparaben Concentration | |
| Density | 1.0514 g/mL |

To a side vessel, propylene glycol was added. Methylparaben was added and the solution was mixed until dissolved. Propylparaben was added and the solution was mixed until dissolved. Glycerin was added and the solution was mixed for at least 15 min until miscible. Xanthan gum was added and the solution was mixed for at least 10 min to disperse xanthan gum. Magnesium aluminometasilicate was added and the solution was mixed for at least 10 min to disperse.

To a main vessel, naproxen sodium was dissolved in 500 mL of water. Cholestyramine resin was added and the solution was mixed for at least 1 hr. Sucralose was added and the solution was mixed for at least 30 min to dissolve. The contents of the side vessel were transferred to the main vessel and the solution was mixed for at least 30 min to dissolve. Grape flavor was added and the solution was mixed for at least 15 min to dissolve. Water was added up to final batch weight, and the batch was mixed for at least 1 hr. The total batch mixing time was approximately 5 hrs, after which a sample was withdrawn from the batch for analysis of final unbound naproxen sodium content, and final unbound propylparaben content.

After approximately 5 hrs of mixing, there was 2.00 mg/mL of unbound naproxen sodium, 2.75 mg/mL of unbound methylparaben, and 0.78 mg/mL of unbound propylparaben in Formulation 6. Formulation 6 possessed a desirable level of grittiness, lacked undesirable chemesthetic effects, consistent with the unbound naproxen sodium being below 2 mg/mL, and had a desirable taste profile.

Further, Formulation 6 was evaluated with TAMC testing and total yeast and mold count testing in accordance with USP <61> and AET in accordance with USP <51>. Formulation 6 passed both TAMC testing and total yeast and mold count testing, as well as AET at both the 14 day and 28 day intervals.

### Example 24

The results from Example 23 demonstrated a formulation having a desirable mouth feel and taste profile, lacking chemesthetic effects, and capable of passing microbial testing (USP <61> and USP <51>) can be produced by omitting polysorbate 80 and including only a combination of methylparaben and propylparaben in place of polysorbate 80. Notably, both Formulation 5 and Formulation 6 contained less total preservative than Formulations 1-4.

Formulation 5 was selected for large scale batch production because, compared to Formulation 6, Formulation 5 had: (i) a lower concentration of unbound naproxen sodium (1.96 mg/mL for Formulation 5 compared to 2.00 for Formulation 6); (ii) a lower concentration of total parabens (0.20% (w/v) for Formulation 5 compared to 0.40% (w/v) for Formulation 6); (iii) a lower concentration of unbound methylparaben (1.34 mg/mL for Formulation 5 compared to 2.75 mg/mL for Formulation 6); and (iv) a lower concentration of unbound propylparaben (0.38 mg/mL for Formulation 5 compared to 0.78 mg/mL for Formulation 6).

Following large scale batch production of Formulation 5, the product (“Formulation 5-LS”) was aliquoted into separate 473 mL containers and long-term stability evaluation was performed under three conditions: (i) 25° C. (at ambient relative humidity) for 12 months; (ii) 40° C. (at ambient relative humidity); and (iii) 60° C. (at ambient relative humidity) for 6 weeks. In addition to long-term stability evaluation microbial testing according to USP <51>, USP <60>, USP <61>, and USP <62> was performed on Formulation 5-LS following batch production and after 6 months of storage at 40° C. (at ambient relative humidity).

### Microbial Testing

Following batch production, Formulation 5-LS was subjected to microbial testing according to USP <51> (at both 14 day and 28 day intervals), USP <60>, USP <61>, and USP <62> and passed all tests. After 6 months of storage at 40° C. (at ambient relative humidity), Formulation 5-LS was subjected to microbial testing according to USP <51> (at both the 14 day and 28 day interval), USP <60>, USP <61>, and USP <62> and passed all tests.

These results indicate that Formulation 5-LS possessed a robust preservative system that effectively prevented microbial growth.

### Impurity Testing

Following batch production, Formulation-5LS was periodically tested for the presence of impurities: (i) Naproxen Related Compound A (“NRC A”) with an acceptance criteria of no more than (“NMT”) 0.2%; (ii) Naproxen Related Compound K (“NRC K”) with an acceptance criteria of NMT 0.2%; (iii) Naproxen Related Compound L (“NRC L”) with an acceptance criteria of NMT 0.2%; (iv) Individual Unspecified Impurities (“IUI”) with an acceptance criteria of NMT 0.2%; and (v) Total Impurities with an acceptance criteria of NMT 1.5%.

Initial analysis of Formulation-5LS demonstrated that none of the foregoing impurities were detected.

For storage at 25° C. (at ambient relative humidity), Formulation-5LS was sampled after 3 months of storage, after 6 months of storage, and after 9 months of storage, and none of the foregoing impurities were detected. Formulation-5LS will be sampled after 12 months of storage, and none of the foregoing impurities will be detected.

For storage at 40° C. (at ambient relative humidity), Formulation-5LS was sampled after 1 month of storage, after 6 weeks of storage, after 2 months of storage, after 3 months of storage, and after 6 months of storage, and none of the foregoing impurities were detected.

For storage at 60° C. (at ambient relative humidity), Formulation-5LS was sampled after 2 weeks of storage, after 1 month of storage, and after 6 weeks of storage, storage, and none of the foregoing impurities were detected after 2 weeks of storage. After 1 month of storage, 0.06% of IUI was detected, and after 6 weeks of storage, 0.05% of IUI was detected.

Formulation 5-LS satisfied the acceptance criteria for all storage conditions, which indicates the formulation possessed a robust stability profile that prevented (or significantly limited) the formation of impurities during storage.

### Redispersibility Testing

Following batch production, Formulation-5LS was periodically subjected to redispersibility to ensure homogeneity of naproxen sodium within the formulation during storage. The acceptance criteria for redispersibility testing was NMT 5.0% difference between the Naproxen Sodium Assay (explained below) of the top and bottom of aliquoted samples.

Initial analysis of the Formulation-5LS batch demonstrated a Naproxen Sodium Assay of 97.4% at the top of the batch and a Naproxen Sodium Assay of 97.5% at the bottom of the batch, for a difference of 0.1%. Initial analysis of the Formulation-5LS aliquoted samples demonstrated a Naproxen Sodium Assay of 97.7% at the top of the batch and a Naproxen Sodium Assay of 98.5% at the bottom of the batch, for a difference of 0.8%.

For storage at 25° C. (at ambient relative humidity), Formulation-5LS was sampled after 3 months of storage, after 6 months of storage, and after 9 months of storage. After 3 months of storage, Formulation-5LS demonstrated a Naproxen Sodium Assay of 97.2% at the top of the batch and a Naproxen Sodium Assay of 97.4% at the bottom of the batch, for a difference of 0.2%. After 6 months of storage, Formulation-5LS demonstrated a Naproxen Sodium Assay of 99.4% at the top of the batch and a Naproxen Sodium Assay of 96.0% at the bottom of the batch, for a difference of 3.4%. After 9 months of storage, Formulation-5LS demonstrated a Naproxen Sodium Assay of 97.8% at the top of the batch and a Naproxen Sodium Assay of 97.6% at the bottom of the batch, for a difference of 0.2%. Formulation-5LS will be sampled after 12 months of storage, and will show a difference of NMT 5.0%.

For storage at 40° C. (at ambient relative humidity), Formulation-5LS was sampled after 1 month of storage, after 2 months of storage, after 3 months of storage, and after 6 months of storage. After 1 month of storage, Formulation-5LS demonstrated a Naproxen Sodium Assay of 97.9% at the top of the batch and a Naproxen Sodium Assay of 97.7% at the bottom of the batch, for a difference of 0.2%. After 2 months of storage, Formulation-5LS demonstrated a Naproxen Sodium Assay of 97.8% at the top of the batch and a Naproxen Sodium Assay of 97.5% at the bottom of the batch, for a difference of 0.3%. After 3 months of storage, Formulation-5LS demonstrated a Naproxen Sodium Assay of 97.8% at the top of the batch and a Naproxen Sodium Assay of 96.8% at the bottom of the batch, for a difference of 1.0%. After 6 months of storage, Formulation-5LS demonstrated a Naproxen Sodium Assay of 98.3% at the top of the batch and a Naproxen Sodium Assay of 99.1% at the bottom of the batch, for a difference of 0.8%.

For storage at 60° C. (at ambient relative humidity), Formulation-5LS was sampled after 2 weeks of storage, after 1 month of storage, and after 6 weeks of storage. After 2 weeks of storage, Formulation-5LS demonstrated a Naproxen Sodium Assay of 95.1% at the top of the batch and a Naproxen Sodium Assay of 95.6% at the bottom of the batch, for a difference of 0.5%. After 1 month of storage, Formulation-5LS demonstrated a Naproxen Sodium Assay of 98.0% at the top of the batch and a Naproxen Sodium Assay of 97.4% at the bottom of the batch, for a difference of 0.6%. After 6 weeks of storage, Formulation-5LS demonstrated a Naproxen Sodium Assay of 99.0% at the top of the batch and a Naproxen Sodium Assay of 98.0% at the bottom of the batch, for a difference of 1.0%.

Formulation 5-LS satisfied the acceptance criteria for all storage conditions, which indicates the formulation possessed a stability profile that maintained the homogeneity of the naproxen sodium concentration within the formulation during storage.

### pH Testing

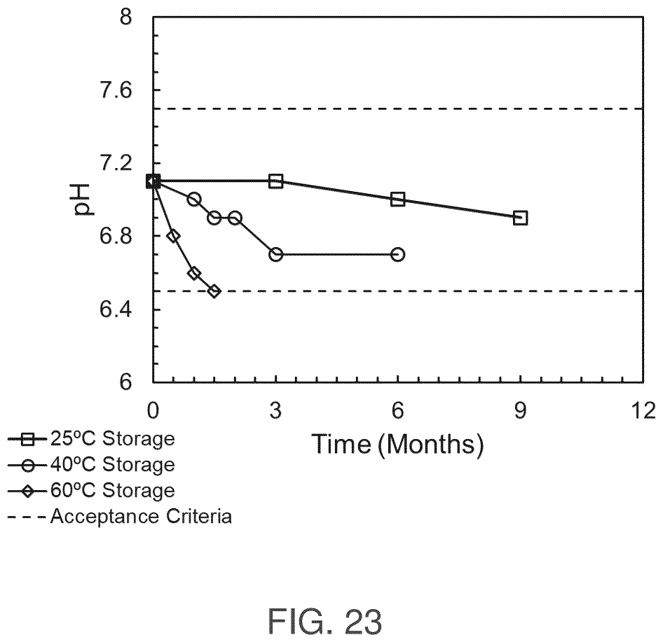

Following batch production, Formulation-5LS was periodically subjected to pH testing according to USP <791> to ensure the formulation remained within the acceptance criteria during storage. The results of pH testing are shown in FIG. 23. The acceptance criteria for pH testing was pH 6.5 to pH 7.5 (FIG. 23, dashed lines).

Initial analysis of Formulation-5LS demonstrated a pH of 7.1.

For storage at 25° C. (at ambient relative humidity), Formulation-5LS was sampled after 3 months of storage, after 6 months of storage, and after 9 months of storage. After 3, 6, and 9 months of storage, Formulation-5LS demonstrated a pH of 7.1, 7.0, and 6.9, respectively (FIG. 23, square series). Formulation-5LS will be sampled after 12 months of storage, and will show pH value of between 6.5 and 7.5.

For storage at 40° C. (at ambient relative humidity), Formulation-5LS was sampled after 1 month of storage, after 6 weeks (1.5 months) of storage, after 2 months of storage, after 3 months of storage, and after 6 months of storage. After 1, 1.5, 2, 3, and 6 months of storage, Formulation-5LS demonstrated a pH of 7.0, 6.9, 6.9, 6.7, and 6.7, respectively (FIG. 23, circle series).

For storage at 60° C. (at ambient relative humidity), Formulation-5LS was sampled after 2 weeks of storage, after 1 month (4 weeks) of storage, and after 6 weeks of storage. After 2, 4, and 6 weeks of storage, Formulation-5LS demonstrated a pH of 6.8, 6.6, and 6.5, respectively (FIG. 23, diamond series).

Formulation-5LS satisfied the acceptance criteria for all storage conditions. While a general trend towards acidic pH was observed across all storage conditions, that evolution towards acidic pH was temperature dependent, and stronger/faster at elevated temperatures and not as strong under ambient storage. Given the slower rate of acidification at ambient storage, these results indicate the formulation would have a stable shelf life that exceeds at least one year (12 months).

### Naproxen Sodium Assay Testing

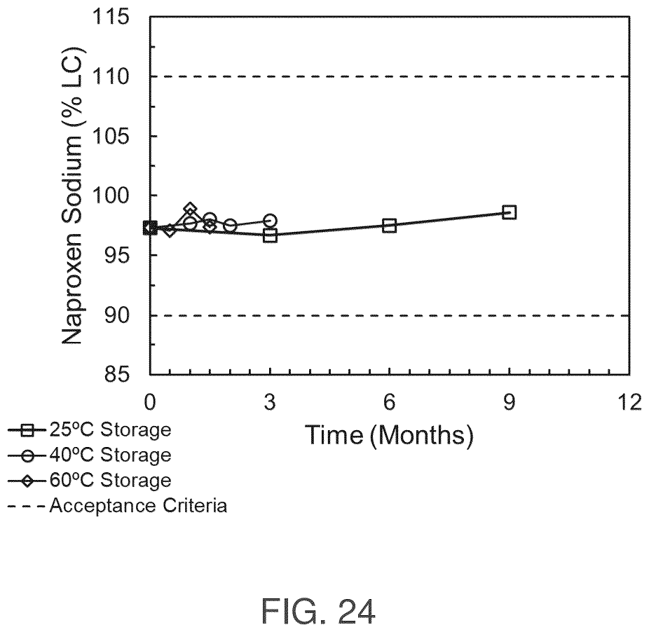

Following batch production, Formulation-5LS was periodically subjected to Naproxen Sodium Assay testing to ensure the formulation remained within the acceptance criteria during storage. The results of Naproxen Sodium Assay testing are shown in FIG. 24. The acceptance criteria for Naproxen Sodium Assay testing was 90% to 110% of the label claim (“% LC”) (FIG. 24, dashed lines).

Initial analysis of Formulation-5LS demonstrated a value for naproxen sodium of 97.3% LC.

For storage at 25° C. (at ambient relative humidity), Formulation-5LS was sampled after 3 months of storage, after 6 months of storage, and after 9 months of storage. After 3, 6, and 9 months of storage, Formulation-5LS demonstrated a value for naproxen sodium of 96.7% LC, 97.5% LC, and 98.6% LC, respectively (FIG. 24, square series). Formulation-5LS will be sampled after 12 months of storage, and will show value for naproxen sodium of between 90% LC and 110% LC.

For storage at 40° C. (at ambient relative humidity), Formulation-5LS was sampled after 1 month of storage, after 6 weeks (1.5 months) of storage, after 2 months of storage, after 3 months of storage, and after 6 months of storage. After 1, 1.5, 2, 3, and 6 months of storage, Formulation-5LS demonstrated a value for naproxen sodium of 97.7% LC, 98.0% LC, 97.5% LC, 97.9% LC, and 99.6% LC, respectively (FIG. 24, circle series).

For storage at 60° C. (at ambient relative humidity), Formulation-5LS was sampled after 2 weeks of storage, after 1 month (4 weeks) of storage, and after 6 weeks of storage. After 2, 4, and 6 weeks of storage, Formulation-5LS demonstrated a value for naproxen sodium of 97.1% LC, 98.9% LC, and 97.4% LC, respectively (FIG. 24, diamond series).

Formulation-5LS satisfied the acceptance criteria for all storage conditions, which indicates the formulation possessed a robust stability profile that maintained the potency/amount of naproxen sodium within the formulation during storage.

### Methylparaben Assay Testing

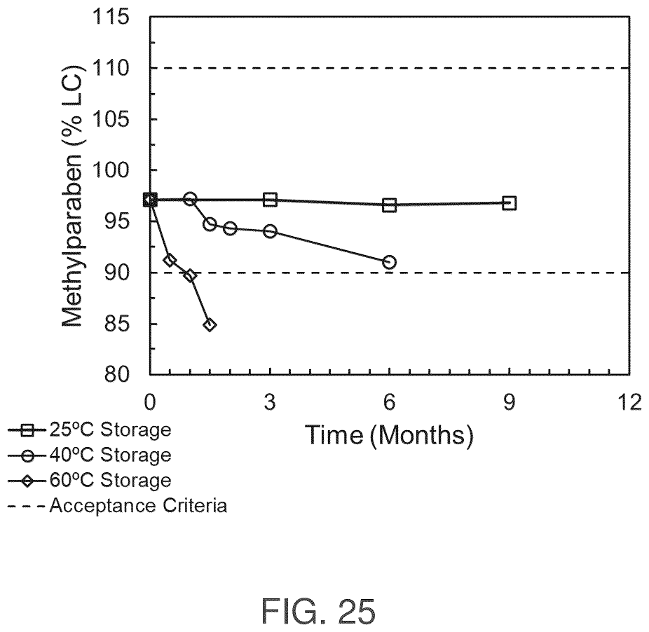

Following batch production, Formulation-5LS was periodically subjected to Methylparaben Assay testing to ensure the formulation remained within the acceptance criteria during storage. The results of Methylparaben Assay testing are shown in FIG. 25. The acceptance criteria for Methylparaben Assay testing was 90% LC to 110% LC (FIG. 25, dashed lines).

Initial analysis of Formulation-5LS demonstrated a value for methylparaben of 97.1% LC.

For storage at 25° C. (at ambient relative humidity), Formulation-5LS was sampled after 3 months of storage, after 6 months of storage, and after 9 months of storage. After 3, 6, and 9 months of storage, Formulation-5LS demonstrated a value for methylparaben of 97.1% LC, 96.6% LC, and 96.8% LC, respectively (FIG. 25, square series). Formulation-5LS will be sampled after 12 months of storage, and will show value for methylparaben of between 90% LC and 110% LC.

For storage at 40° C. (at ambient relative humidity), Formulation-5LS was sampled after 1 month of storage, after 6 weeks (1.5 months) of storage, after 2 months of storage, after 3 months of storage, and after 6 months of storage. After 1, 1.5, 2, 3, and 6 months of storage, Formulation-5LS demonstrated a value for methylparaben of 97.2% LC, 94.7% LC, 94.3% LC, 94.0% LC, and 91.0% LC, respectively (FIG. 25, circle series).

For storage at 60° C. (at ambient relative humidity), Formulation-5LS was sampled after 2 weeks of storage, after 1 month (4 weeks) of storage, and after 6 weeks of storage. After 2, 4, and 6 weeks of storage, Formulation-5LS demonstrated a value for methylparaben of 91.2% LC, 89.7% LC, and 84.9% LC, respectively (FIG. 25, diamond series).

Formulation-5LS satisfied the acceptance criteria for all storage conditions except for storage at 60° C. (at ambient relative humidity). While deviation outside of the acceptance criteria occurred for this single storage condition, no such similar deviation (or general trend of methylparaben loss) was observed under ambient storage (i.e., 25° C.), which indicates the amount of methylparaben would be stable during the shelf life of the formulation, and thus the robust preservative and stability profile of the formulation would persist through its shelf life.

### Propylparaben Assay Testing

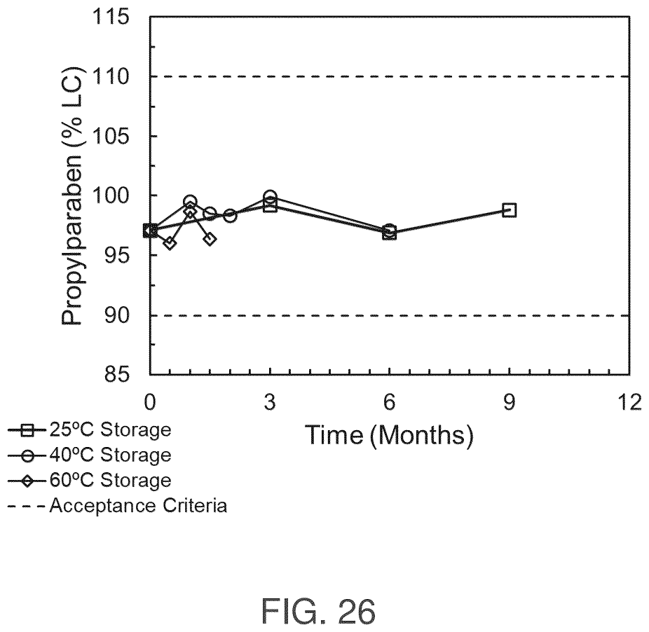

Following batch production, Formulation-5LS was periodically subjected to Propylparaben Assay testing to ensure the formulation remained within the acceptance criteria during storage. The results of Propylparaben Assay testing are shown in FIG. 26. The acceptance criteria for Propylparaben Assay testing was 90% LC to 110% LC (FIG. 26, dashed lines).

Initial analysis of Formulation-5LS demonstrated a value for propylparaben of 97.1% LC.

For storage at 25° C. (at ambient relative humidity), Formulation-5LS was sampled after 3 months of storage, after 6 months of storage, and after 9 months of storage. After 3, 6, and 9 months of storage, Formulation-5LS demonstrated a value for propylparaben of 99.2% LC, 96.9% LC, and 98.8% LC, respectively (FIG. 26, square series). Formulation-5LS will be sampled after 12 months of storage, and will show value for propylparaben of between 90% LC and 110% LC.

For storage at 40° C. (at ambient relative humidity), Formulation-5LS was sampled after 1 month of storage, after 6 weeks (1.5 months) of storage, after 2 months of storage, after 3 months of storage, and after 6 months of storage. After 1, 1.5, 2, 3, and 6 months of storage, Formulation-5LS demonstrated a value for propylparaben of 99.5% LC, 98.5% LC, 98.3% LC, 99.0% LC, and 97.1% LC, respectively (FIG. 26, circle series).

For storage at 60° C. (at ambient relative humidity), Formulation-5LS was sampled after 2 weeks of storage, after 1 month (4 weeks) of storage, and after 6 weeks of storage. After 2, 4, and 6 weeks of storage, Formulation-5LS demonstrated a value for propylparaben of 96.0% LC, 98.7% LC, and 96.4% LC, respectively (FIG. 26, diamond series).

Formulation-5LS satisfied the acceptance criteria for all storage conditions, which indicates the amount of propylparaben would be stable during the shelf life of the formulation, and thus the robust preservative and stability profile of the formulation would persist through its shelf life.

### Viscosity Testing

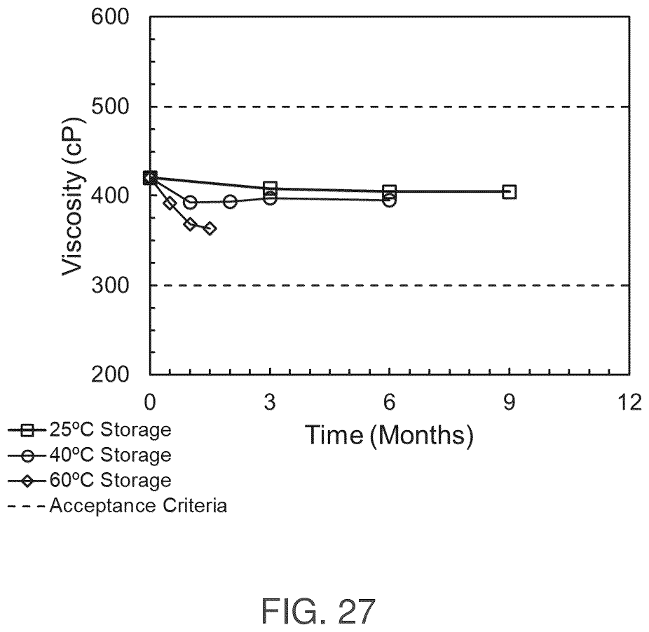

Following batch production, Formulation-5LS was periodically subjected to viscosity testing according to USP <912> to ensure the formulation remained within the acceptance criteria during storage. The results of viscosity testing are shown in FIG. 27. The acceptance criteria for viscosity testing was a viscosity of 300 cP to 500 cP (FIG. 27, dashed lines).

Initial analysis of Formulation-5LS demonstrated a viscosity of 420.0 cP.

For storage at 25° C. (at ambient relative humidity), Formulation-5LS was sampled after 3 months of storage, after 6 months of storage, and after 9 months of storage. After 3, 6, and 9 months of storage, Formulation-5LS demonstrated a viscosity of 408.0 cP, 404.4 cP, and 404.4 cP, respectively (FIG. 27, square series). Formulation-5LS will be sampled after 12 months of storage, and will show viscosity of between 300 cP and 500 cP.

For storage at 40° C. (at ambient relative humidity), Formulation-5LS was sampled after 1 month of storage, after 2 months of storage, after 3 months of storage, and after 6 months of storage. After 1, 2, 3, and 6 months of storage, Formulation-5LS demonstrated a viscosity of 392.4 cP, 393.6 cP, 397.2 cP, and 394.8 cP, respectively (FIG. 27, circle series).

For storage at 60° C. (at ambient relative humidity), Formulation-5LS was sampled after 2 weeks of storage, after 1 month (4 weeks) of storage, and after 6 weeks of storage. After 2, 4, and 6 weeks of storage, Formulation-5LS demonstrated a viscosity of 391.6 cP, 368.4 cP, and 363.6 cP, respectively (FIG. 27, diamond series).

Formulation-5LS satisfied the acceptance criteria for all storage conditions. While a general trend towards less viscosity was observed across all storage conditions, that evolution towards less viscosity was temperature dependent, and stronger/faster at elevated temperatures and not as strong under ambient storage. Given the slower rate transition to lower viscosity at ambient storage, these results indicate the formulation would have a stable shelf life that exceeds at least one year (12 months).

### Dissolution Testing

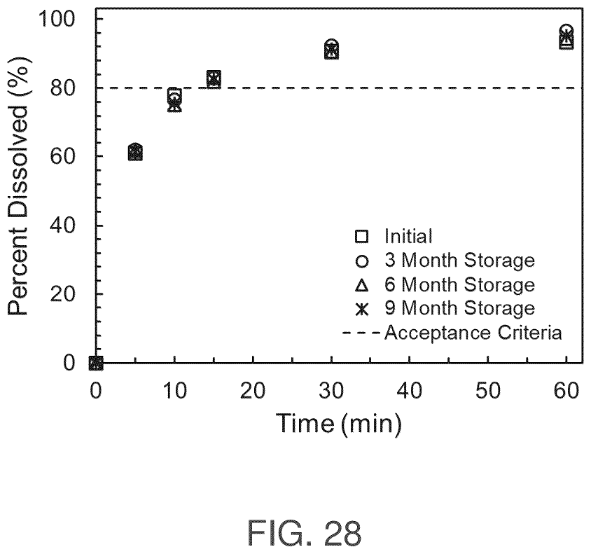

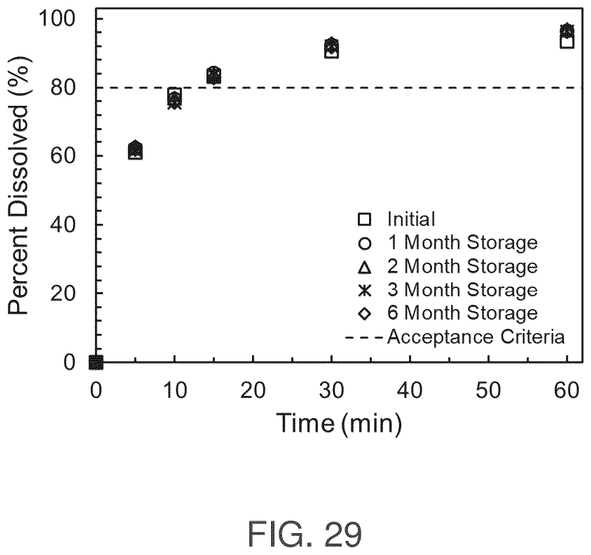

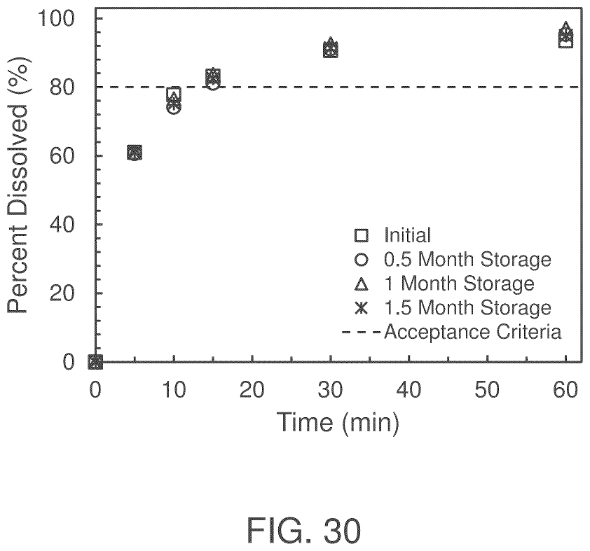

Following batch production, Formulation-5LS was periodically subjected to dissolution testing to ensure the formulation remained within the acceptance criteria during storage. The results of viscosity testing are shown in FIG. 28 (for storage at 25° C.), FIG. 29 (for storage at 40° C.), and FIG. 30 (for storage at 60° C.). Across all storage conditions, the acceptance criteria for dissolution testing was no less than (“NLT”) 80% dissolved at 60 min (FIGS. 28-30, dashed lines). For all dissolution testing, reported values reflect the average of 6 replicates.

Initial analysis of Formulation-5LS demonstrated a dissolution profile containing naproxen sodium release values of 61.0% at 5 min, 77.8% at 10 min, 83.2% at 15 min, 90.6% at 30 min, and 93.4% at 60 min (FIGS. 28-30, square series).

For storage at 25° C. (at ambient relative humidity), Formulation-5LS was sampled after 3 months of storage, after 6 months of storage, and after 9 months of storage. After 3 months of storage, the formulation demonstrated a dissolution profile containing naproxen sodium release values of 62.0% at 5 min, 76.5% at 10 min, 82.9% at 15 min, 92.3% at 30 min, and 96.5% at 60 min (FIG. 28, circle series). After 6 months of storage, the formulation demonstrated a dissolution profile containing naproxen sodium release values of 61.8% at 5 min, 75.2% at 10 min, 81.9% at 15 min, 90.5% at 30 min, and 94.3% at 60 min (FIG. 28, triangle series). After 9 months of storage, the formulation demonstrated a dissolution profile containing naproxen sodium release values of 61.4% at 5 min, 75.5% at 10 min, 82.5% at 15 min, 91.3% at 30 min, and 95.1% at 60 min (FIG. 28, asterisk series). Formulation-5LS will be sampled after 12 months of storage, and will satisfy the acceptance criteria and demonstrate a dissolution profile containing naproxen sodium release values in line with the foregoing values at 3, 6, and/or 9 months of storage.

For storage at 40° C. (at ambient relative humidity), Formulation-5LS was sampled after 1 month of storage, after 2 months of storage, after 3 months of storage, and after 9 months of storage. After 1 month of storage, the formulation demonstrated a dissolution profile containing naproxen sodium release values of 62.3% at 5 min, 76.6% at 10 min, 84.3% at 15 min, 92.3% at 30 min, and 96.4% at 60 min (FIG. 29, circle series). After 2 months of storage, the formulation demonstrated a dissolution profile containing naproxen sodium release values of 62.3% at 5 min, 76.8% at 10 min, 83.9% at 15 min, 92.8% at 30 min, and 96.9% at 60 min (FIG. 29, triangle series). After 3 months of storage, the formulation demonstrated a dissolution profile containing naproxen sodium release values of 61.7% at 5 min, 75.6% at 10 min, 83.7% at 15 min, 92.0% at 30 min, and 96.3% at 60 min (FIG. 29, asterisk series). After 6 months of storage, the formulation demonstrated a dissolution profile containing naproxen sodium release values of 62.7% at 5 min, 75.6% at 10 min, 82.8% at 15 min, 91.8% at 30 min, and 96.2% at 60 min (FIG. 29, diamond series).

For storage at 60° C. (at ambient relative humidity), Formulation-5LS was sampled after 2 weeks (0.5 months) of storage, after 1 month of storage, and after 6 weeks (1.5 months) of storage. After 0.5 months of storage, the formulation demonstrated a dissolution profile containing naproxen sodium release values of 60.6% at 5 min, 74.1% at 10 min, 81.1% at 15 min, 91.1% at 30 min, and 95.3% at 60 min (FIG. 30, circle series). After 1 month of storage, the formulation demonstrated a dissolution profile containing naproxen sodium release values of 61.0% at 5 min, 76.6% at 10 min, 83.7% at 15 min, 92.6% at 30 min, and 97.1% at 60 min (FIG. 30, triangle series). After 1.5 months of storage, the formulation demonstrated a dissolution profile containing naproxen sodium release values of 61.0% at 5 min, 75.2% at 10 min, 82.5% at 15 min, 91.4% at 30 min, and 95.0% at 60 min (FIG. 30, asterisk series).

Formulation-5LS satisfied the acceptance criteria for all storage conditions. Notably, the dissolution testing demonstrated that independent of storage time and storage temperature, the formulation resulted in substantially similar dissolution profiles, which indicates the formulation possessed a robust stability profile that ensured a consistent potency/amount of naproxen sodium was released from the formulation within a specified time frame.

The present disclosure is not to be limited in terms of the particular embodiments described in this application, which are intended as illustrations of various aspects of the present disclosure. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The terminology used herein is for the purpose of describing particular embodiments only and is not intended to be limiting unless otherwise set forth herein.

1. An IEX-NSAID complex, wherein the IEX-NSAID complex comprises a nonsteroidal anti-inflammatory drug (NSAID) or salt thereof bound to an ion-exchange (IEX) resin.

2. The IEX-NSAID complex of claim 1, wherein the NSAID or salt thereof comprises naproxen or a salt thereof.

3. The IEX-NSAID complex of claim 2, wherein the NSAID or salt thereof comprises an alkali salt of naproxen.

4. The IEX-NSAID complex of claim 3, wherein the alkali salt of naproxen comprises naproxen sodium.

5. The IEX-NSAID complex of claim 1, wherein the IEX resin comprises an anionic exchange resin.

6. The IEX-NSAID complex of claim 5, wherein the anionic exchange resin comprises a cholestyramine resin.

7. The IEX-NSAID complex of claim 1, wherein the IEX-NSAID complex comprises about 46% NSAID or salt thereof by weight to about 52% NSAID or salt thereof by weight.

8. The IEX-NSAID complex of claim 1, wherein the IEX-NSAID complex releases at least 50% of bound NSAID or salt thereof within 10 minutes during a dissolution test.

9. The IEX-NSAID complex of claim 8, wherein the IEX-NSAID complex releases at least 80% of bound NSAID or salt thereof within 10 minutes during the dissolution test.

10. The IEX-NSAID complex of claim 1, wherein the IEX-NSAID complex releases at least 75% of bound NSAID or salt thereof within 30 minutes during a dissolution test.

11. The IEX-NSAID complex of claim 10, wherein the IEX-NSAID complex releases at least 85% of bound NSAID or salt thereof within 30 minutes during the dissolution test.

12. The IEX-NSAID complex of claim 1, wherein the IEX-NSAID complex releases at least 80% of bound NSAID or salt thereof within 60 minutes during a dissolution test.

13. The IEX-NSAID complex of claim 12, wherein the IEX-NSAID complex releases at least 90% of bound NSAID or salt thereof within 60 minutes during the dissolution test.

14. A liquid dosage form, the liquid dosage form comprising an amount of an IEX-NSAID complex, wherein the IEX-NSAID complex comprises a nonsteroidal anti-inflammatory drug (NSAID) or salt thereof bound to an ion-exchange (IEX) resin, and optionally one or more excipients selected from the group consisting of a sweetener, a thickener, a preservative, a flavorant, and any combination thereof.

15. The liquid dosage form of claim 14, wherein the NSAID or salt thereof comprises naproxen or a salt thereof.

16. The liquid dosage form of claim 15, wherein the NSAID or salt thereof comprises an alkali salt of naproxen.

17. The liquid dosage form of claim 16, wherein the alkali salt of naproxen comprises naproxen sodium.

18. The liquid dosage form of claim 14, wherein the IEX resin comprises an anionic exchange resin.

19. The liquid dosage form of claim 18, wherein the anionic exchange resin comprises cholestyramine.
